(19) United States
(12) Patent Application Publication
(10) Pub. No.: US 2023/0051885 A1
(43) Pub. Date: Feb. 16, 2023

(54) Systems and Methods for Producing Efficacious Regulatory T Cells

(71) Applicant: H. LEE MOFFITT CANCER CENTER AND RESEARCH INSTITUTE INC., Tampa, FL (US)

(72) Inventor: Marco L. Davila, Tampa, FL (US)

(21) Appl. No.: 17/757,475

(22) PCT Filed: Dec. 17, 2020
(86) PCT No.: PCT/US2020/065660

§ 371 Date: Jun. 16, 2022

Related U.S. Application Data
(60) Provisional application No. 62/949,527, filed on Dec. 18, 2019.

(51) Int. Cl. C12N 5/0783; C12N 15/86; C07K 16/28

(52) U.S. Cl. CPC C12N 5/0637; C12N 15/86; C07K 16/2803; C07K 2317/622; C07K 2317/565

(57) ABSTRACT

Regulatory T cells (T_{reg} cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Two major classes of CD4⁺ T_{reg} cells have been described—naturally occurring T_{reg} cells and adaptive T_{reg} cells. Disclosed herein are methods for producing efficacious CAR T_{reg} cells in a GMP-scalable system.

### CROSS-REFERENCE TO RELATED APPLICATIONS

This application claims benefit of U.S. Provisional Application No. 62/949,527, filed Dec. 18, 2019, which is hereby incorporated herein by reference in its entirety.

### SEQUENCE LISTING

This application contains a sequence listing filed in electronic form as an ASCII.txt file entitled “320803_2490_Sequence_Listing_ST25” created on Dec. 17, 2020. The content of the sequence listing is incorporated herein in its entirety.

### BACKGROUND

Graft-versus-host disease (GVHD) is a significant cause of non-relapse mortality after allogeneic hematopoietic cell transplantation (allo-HCT). Existing strategies to prevent and treat GVHD are incomplete, where a significant portion of allo-HCT recipients developed this complication. Despite this, one such therapy has emerged involving the use of regulatory T (T_{reg}) cells to control GVHD. Regulatory T cells (T_{reg} cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. The use of natural T_{reg} cells and artificial CAR-T_{reg} cells have shown promise to reduce GVHD. However, there is a need in the art for improved methods for producing these cells for use in adoptive cell therapy.

### SUMMARY

Disclosed herein are methods for producing efficacious T_{reg} cells, such as CAR T_{reg} cells, in a GMP-scalable system. In a first embodiment, the method involves the use of artificial antigen presenting cells (AAPC) engineered activate and expand T_{reg} cells. A rapid and efficacious ex vivo production system for Tregs allows successful clinical translation of Tregs for patients with GVHD or autoimmunity. In some embodiments, the AAPCs provide primary activation via CD3ζ and co-stimulation via CD28 (FIG. 1). This can be accomplished by engineering an antigen presenting cell line, such as a K562 cell line, to express anti-CD3 and anti-CD28 scFv's, as well as the 41BB ligand (41BBL), which enhances Treg expansion. In some embodiments, the AAPCs are further modified to express the IL15 receptor (IL15R), which provide IL15 costimulation, thereby promoting T_{reg} expansion. In some embodiments, the AAPC is modified to express the heparin-binding domain (HBD), which binds retrovirus, to facilitate retroviral gene transfer of the CAR T_{reg} cells (FIG. 4). The AAPC that ligate CD3 and bind virus via HBD support efficient gene transfer of primary human T cells (FIG. 4).

T_{reg} cells express the biomarkers CD4, FOXP3, and CD25 and are thought to be derived from the same lineage as naïve CD4 cells. Because effector T cells also express CD4 and CD25, Tregs are very difficult to effectively discern from effector CD4+. In a second embodiment, the method involves isolating CD4/CD25 T cells from a subject and inducing T_{reg} differentiation in these cells through gene-modification. In some embodiments, the isolated cells are genetically engineered to express IL2RB, FOXP3, SOCS, or any combination thereof, which regulate T_{reg} differentiation.

In some embodiments, the CAR T_{reg} cells express a chimeric antigen receptor (CAR) targeting CD83. Also disclosed herein is a method of suppressing alloreactive donor cells in a subject receiving transplanted donor hematopoietic cells or even solid organ allografts that involves administering to the subject an effective amount of CAR T_{reg} cells produced by the disclosed methods genetically modified to express a chimeric antigen receptor (CAR) targeting CD83. CD83 is differentially expressed on alloreactive T cells, but not T_{reg} cells. The CD83 CAR T_{reg} can target T cells that cause GVHD and spare GVL. Even when donors are fully HLA matched, the minor HLA disparity or the presence of H-Y antigens are sufficient to cause GVHD. Additionally, not all donors are HLA-matched to recipients, such as HLA-DP mismatch, which can also result in severe GVHD. A unique benefit of the CD83 CAR T_{reg} is that it can suppress rather than kill alloreactive T cells, to avoid lymphopenia.

The anti-CD83 binding agent is in some embodiments an antibody fragment that specifically binds CD83. For example, the antigen binding domain can be a Fab or a single-chain variable fragment (scFv) of an antibody that specifically binds CD83. The anti-CD83 binding agent is in some embodiments an aptamer that specifically binds CD83. For example, the anti-CD83 binding agent can be a peptide aptamer selected from a random sequence pool based on its ability to bind CD83. The anti-CD83 binding agent can also be a natural ligand of CD83, or a variant and/or fragment thereof capable of binding CD83.

In some embodiments, the anti-CD83 scFv can comprise a variable heavy (V_{H}) domain having CDR1, CDR2 and CDR3 sequences and a variable light (V_{L}) domain having CDR1, CDR2 and CDR3 sequences.

For example, in some embodiments, the CDR1 sequence of the V_{H} domain comprises the amino acid sequence GFSITTGGYWWT (SEQ ID NO:1), SDGIS (SEQ ID NO:7), or SNAMI (SEQ ID NO:13); CDR2 sequence of the V_{H} domain comprises the amino acid sequence GYIFSSGNTNYNPSIKS (SEQ ID NO:2), IISSGGNTYYASWAKG (SEQ ID NO:8), or AMDSNSRTYYATWAKG (SEQ ID NO:14); CDR3 sequence of the V_{H} domain comprises the amino acid sequence CARAYGKLGFDY (SEQ ID NO:3), VVGGTYSI (SEQ ID NO:9), or GDGGSSDYTEM (SEQ ID NO:15); CDR1 sequence of the V_{L} comprises the amino acid sequence TLSSQHSTYTIG (SEQ ID NO:4), QSSQSVYNNDFLS (SEQ ID NO:10), or QSSQSVYGNNELS (SEQ ID NO:16); CDR2 sequence of the V_{L} domain comprises the amino acid sequence VNSDGSHSKGD (SEQ ID NO:5), YASTLAS (SEQ ID NO:11), or QASSLAS (SEQ ID NO:17); and CDR3 sequence of the V_{L} domain comprises the amino acid sequence GSSDSSGYV (SEQ ID NO:6), TGTYGNSAWYEDA (SEQ ID NO:12), or LGEYSISADNH (SEQ ID NO:18).

For example, in some embodiments, the CDR1 sequence of the V_{H} domain comprises the amino acid sequence GFSITTGGYWWT (SEQ ID NO:1), CDR2 sequence of the V_{H} domain comprises the amino acid sequence GYIFSSGNTNYNPSIKS (SEQ ID NO:2), CDR3 sequence of the V_{H} domain comprises the amino acid sequence CARAYGKLGFDY (SEQ ID NO:3), CDR1 sequence of the V_{L} comprises the amino acid sequence TLSSQHSTYTIG (SEQ ID NO:4), CDR2 sequence of the V_{L} domain comprises the amino acid sequence VNSDGSHSKGD (SEQ ID NO:5), and CDR3 sequence of the V_{L} domain comprises the amino acid sequence GSSDSSGYV (SEQ ID NO:6).

For example, in some embodiments, the CDR1 sequence of the V_{H} domain comprises the amino acid sequence SDGIS (SEQ ID NO:7), CDR2 sequence of the V_{H} domain comprises the amino acid sequence IISSGGNTYYASWAKG (SEQ ID NO:8), CDR3 sequence of the V_{H} domain comprises the amino acid sequence VVGGTYSI (SEQ ID NO:9), CDR1 sequence of the V_{L} comprises the amino acid sequence QSSQS VYNNDFLS (SEQ ID NO:10), CDR2 sequence of the V_{L} domain comprises the amino acid sequence YASTLAS (SEQ ID NO: 11), and CDR3 sequence of the V_{L} domain comprises the amino acid sequence TGTYGNSAWYEDA (SEQ ID NO:12).

For example, in some embodiments, the CDR1 sequence of the V_{H} domain comprises the amino acid sequence SNAMI (SEQ ID NO:13), CDR2 sequence of the V_{H} domain comprises the amino acid sequence AMDSNSRTYYATWAKG (SEQ ID NO:14), CDR3 sequence of the V_{H} domain comprises the amino acid sequence GDGGSSDYTEM (SEQ ID NO:15), CDR1 sequence of the V_{L} comprises the amino acid sequence QSSQSVYGNNELS (SEQ ID NO:16), CDR2 sequence of the V_{L} domain comprises the amino acid sequence QASSLAS (SEQ ID NO:17), and CDR3 sequence of the V_{L} domain comprises the amino acid sequence LGEYSISADNH (SEQ ID NO:18).

In some embodiments, the anti-CD83 scFv V_{H} domain comprises the amino acid sequence:

QVQLKESGPGLVKPSQSLSLTCSVTGFSITTGGYWWTWIRQFPGQKLEWM
GYIFSSGNTNYNPSIKSRISITRDTSKNQFFLQLNSVTTEGDTARYYCAR
AYGKLGFDYWGQGTLVTVSS (SEQ ID NO: 19, VH-GBM00).

In some embodiments, the anti-CD83 scFv V_{L} domain comprises the amino acid sequence:

QPVLTQSPSASASLGNSVKITCTLSSQHSTYTIGWYQQHPDKAPKYVMYV
NSDGSHSKGDGIPDRFSGSSSGAHRYLSISNIQPEDEADYFCGSSDSSGY
VFGSGTQLTVL (SEQ ID NO: 20, VL-GBM00).

In some embodiments, the anti-CD83 scFv V_{H} domain comprises the amino acid sequence:

METGLRWLLLVAVLKGVQCQSVEESGGRLVTPGTPLTLTCTVSGFSLSNN
AINVRQAPGKGLEWIGYIWSGGLTYYANWAEGRFTISKTSTTVDLKMTSP
TIEDTATYFCARGINNSALWGPGTLVTVSSGQPKAPSVFPLAPCCGDTPS
STVTLGCLVKGYLPEPVTVTWNSGTLTNGVRTFPSVRQSSGLYSLSSVVS
VTSSSQPVTCNVAHPATNTKVDKTVAPSTCSKPTCPPPELLGGPSVFIFP
PKPKDTLMISRTPEVTCVVVDVSQDDPEVQFTWYINNEQVRTARPPLREQ
QFNSTIRVVSTLPIAHQDWLRGKEFKCKVHNKALPAPIEKTISKARGQPL
EPKVYTMGPPREELSSRSVSLTCMINGFYPSDISVEWEKNGKAEDNYKTT
PAVLDSDGSYFLYNKLSVPTSEWQRGDVFTCSVMHEALHNHYTQKSISRS
PGK (SEQ ID NO: 21, 20D04).

In some embodiments, the anti-CD83 scFv V_{L} domain comprises the amino acid sequence:

MDMRAPTQLLGLLLLWLPGARCADVVMTQTPASVSAAVGGTVTINCQASE
SISNYLSWYQQKPGQPPKLLIYRTSTLASGVSSRFKGSGSGTEYTLTISG
VQCDDVATYYCQCTSGGKFISDGAAFGGGTEVVVKGDPVAPTVLLFPPSS
DEVATGTVTIVCVANKYFPDVTVTWEVDGTTQTTGIENSKTPQNSADCTY
NLSSTLTLTSTQYNSHKEYTCKVTQGTTSVVQSFSRKNC (SEQ ID
NO: 22, 20D04).

In some embodiments, the anti-CD83 scFv V_{H} domain comprises the amino acid sequence:

METGLRWLLLVAVLKGVQCQSVEESGGRLVTPGTPLTLTCTVSGFTISDY
DLSWRQAPGEGLKYIGFIAIDGNPYYATWAKGRFTISKTSTTVDLKITAP
TTEDTATYFCARGAGDLWGPGTLVTVSSGQPKAPSVFPLAPCCGDTPSST
VTLGCLVKGYLPEPVTVTWNSGTLTNGVRTFPSVRQSSGLYSLSSVVSVT
SSSQPVTCNVAHPATNTKVDKTVAPSTCSKPTCPPPELLGGPSVFIFPPK
PKDTLMISRTPEVTCVVVDVSQDDPEVQFTWYINNEQVRTARPPLREQQF
NSTIRVVSTLPIAHQDWLRGKEFKCKVHNKALPAPIEKTISKARGQPLEP
KVYTMGPPREELSSRSVSLTCMINGFYPSDISVEWEKNGKAEDNYKTTPA
VLDSDGSYFLYNKLSVPTSEWQRGDVFTCSVMHEALHNHYTQKSISRSPG
K (SEQ ID NO: 23, 11G05).

In some embodiments, the anti-CD83 scFv V_{L} domain comprises the amino acid sequence:

MDTREPTQLLGLLLLWLPGARCADVVMTQTPASVSAAVGGTVTINCQSSK
NVYNNNWLSWFQQKPGQPPKLLIYYASTLASGVPSRFRGSGSGTQFTLTI
SDVQCDDAATYYCAGDYSSSSDNGFGGGTEVVVKGDPVAPTVLLFPPSSD
EVATGTVTIVCVANKYFPDVTVTWEVDGTTQTTGIENSKTPQNSADCTYN
LSSTLTLTSTQYNSHKEYTCKVTQGTTSVVQSFSRKNC (SEQ ID NO:
24, 11G05).

In some embodiments, the anti-CD83 scFv V_{H} domain comprises the amino acid sequence:

METGLRWLLLVAVLKGVHCQSVEESGGRLVTPGTPLTLTCTASGFSRSSY
DMSVRQAPGKGLEWWGVISTAYNSHYASWAKGRFTISRTSTTVDLKMTSL
TTEDTATYFCARGGSWLDLWGQGTLVTVSSGQPKAPSVFPLAPCCGDTPS
STVTLGCLVKGYLPEPVTVTWNSGTLTNGVRTFPSVRQSSGLYSLSSVSV
TSSSQPVTCNVAHPATNTKVDKTVAPSTCSKPTCPPPELLGGPSVFIFPP
KPKDTLMISRTPEVTCVVVDVSQDDPEVQFTWYINNEQVRTARPPLREQQ
FNSTIRVVSTLPIAHQDWLRGKEFKCKVHNKALPAPIEKTISKARGQPLE
PKVYTMGPPREELSSRSVSLTCMINGFYPSDISVEWEKNGKAEDNYKTTP
AVLDSDGSYFLYNKLSVPTSEWQRGDVFTCSVMHEALHNHYTQKSISRSP
GK (SEQ ID NO: 25, 14C12).

In some embodiments, the anti-CD83 scFv V_{L} domain comprises the amino acid sequence:

MDXRAPTQLLGLLLLWLPGARCALVMTQTPASVSAAVGGTVTINCQSSQS
VYDNDELSWYQQKPGQPPKLLIYALASKLASGVPSRFKGSGSGTQFALTI
SGVQCDDAATYYCQATHYSSDWYLTFGGGTEVVVKGFPVAPTVLLFPPSS
DEVATGTVTIVCVANKYFPDVTVTWEVDGTTQTTGTENSKTPQNSADCTY
NLSSTLTLTSTQYNSHKEYTCKVTQGTTSVVQSFSRKNC (SEQ ID
NO: 26, 14C12).

In some embodiments, the anti-CD83 scFv V_{H} domain comprises the amino acid sequence:

METGLRWLLLVAVLKGVQCQSVEESGGRLVTPGTPLTLTCTVSGFSLSSY
DMTVRQAPGKGLEWIGIIYASGTTYYANWAKGRFTISKTSTTVDLKVTSP
TIGDTATYFCAREGAGVSMTLWGPGTLVTVSSGQPKAPSVFPLAPCCGDT
PSSTVTLGCLVKGYLPEPVTVTWNSGTLTNGVRTFPSVRQSSGLYSLSSV
SVTSSSQPVTCNVAHPATNTKVDKTVAPSTCSKPTCPPPELLGGPSVFIF
PPKPKDTLMISRTPEVTCVVVDVSQDDPEVQFTWINNEQVRTARPPLREQ
QFNSTIRVVSTLPIAHQDWLRGKEFKCKVHNKALPAPIEKTISKARGQPL
EPKVYTMGPPREELSSRSVSLTCMINGFYPSDISVEWEKNGKAEDNYKTT
PAVLDSDGSYFLYNKLSVPTSEWQRGDVFTCSVMHEALHNHYTQKSISRS
PGK (SEQ ID NO: 27, 020B08).

In some embodiments, the anti-CD83 scFv V_{L} domain comprises the amino acid sequence:

(SEQ ID NO: 28, 020B08)
MDMRAPTQLLGLLLLWLPGARCAYDMTQTPASVEVAVGGTVTIKCQASQSISTYLDWYQQ
KPGQPPKLLIYDASDLASGVPSRFKGSGSGTQFTLTISDLECADAATYYCQQGYTHSNVDN
VFGGGTEVVVKGDPVAPTVLLFPPSSDEVATGTVTIVCVANKYFPDVTVTWEVDGTTQTTG
IENSKTPQNSADCTYNLSSTLTLTSTQYNSHKEYTCKVTQGTTSVVQSFSRKNC

In some embodiments, the anti-CD83 scFv V_{H} domain comprises the amino acid sequence:

(SEQ ID NO: 29, 006G05)
METGLRWLLLVAVLKGVQCQSVEESGGRLVSPGTPLTLTCTASGFSLSSYDMSVWRQAPG
KGLEYIGIISSSGSTYYASWAKGRFTISKTSTTVDLEVTSLTTEDTATYFCSREHAGYSGDTG
HLWGPGTLVTVSSGQPKAPSVFPLAPCCGDTPSSTVTLGCLVKGYLPEPVTVTWNSGTLT
NGVRTFPSVRQSSGLYSLSSVVSVTSSSQPVTCNVAHPATNTKVDKTVAPSTCSKPTCPPP
ELLGGPSVGIGPPKPKDTLMISRTPEVTCVVVDVSQDDPEVQFTWYINNEQVRTARPPLRE
QQFNSTIRVVSTLPIAHQDWLRGKEFKCKVHNKALPAPIEKTISKARGQPLEPKVYTMGPPR
EELSSRSVSLTCMINGFYPSDISVEWEKNGKAEDNYKTTPAVLDSDGSYFLYNKLSVPTSE
WQRGDVFTCSVMHEALHNHYTQKSISRSPGK.

In some embodiments, the anti-CD83 scFv V_{L} domain comprises the amino acid sequence:

(SEQ ID NO: 30, 006G05)
MDMRAPTQLLGLLLLWLPGARCAYDMTQTPASVEVAVGGTVAIKCQASQSVSSYLAWYQQ
KPGQPPKPLIYEASMLAAGVSSRFKGSGSGTDFTLTISDLECDDAATYYCQQGYSISDIDNA
FGGGTEVVVKGDPVAPTVLLFPPSSDEVATGTVTIVCVANKYFPDVTVTWEVDGTTQTTGI
ENSKTPQNSADCTYNLSSTLTLTSTQYNSHKEYTCKVTQGTTSVVQSFSRKNC

In some embodiments, the anti-CD83 scFv V_{H} domain comprises the amino acid sequence:

(SEQ ID NO: 31, 96G08)
METGLRWLLLVAVLKGVQCQSVEESGGRLVTPGTPLTLTCTVSGIDLSSDGISWRQAPGK
GLEWIGIISSGGNTYYASWAKGRFTISRTSTTVDLKMTSLTTEDTATYFCARVVGGTYSIWG
QGTLVTVSSASTKGPSVYPLAPGSAAQTNSMVTLGCLVKGYFPEPVTVTWNSGSLSSGVH
TFPAVLQSDLYTLSSSVTVPSSTWPSETVTCNVAHPASSTKVDKKIVPRDCGCKPCICTVPE
VSSVFIFPPKPDVLTITLTPKVTCVVVDISKDDPEVQFSWFVDDVEVHTAQTQPREEQFNST
FRSVSELPIMHQDWLNGKEFKCRVNSAAFPAPIEKTISKTKGRPKAPQVYTIPPPKEQMAKD
KVSLTCMITDFFPEDITVEWQWNGQPAENYKNTQPIMDTDGSYFVYSKLNVQKSNWEAGN
TFTCSVLHEGLHNHHTEKSLSHSPGK.

In some embodiments, the anti-CD83 scFv V_{L} domain comprises the amino acid sequence:

(SEQ ID NO: 32, 96G08)
MDTRAPTQLLGLLLLWLPGATFAQVLTQTASPVSAPVGGTVTINCQSSQSVYNNDFLSWYQ
QKPGQPPKLLIYYASTLASGVPSRFKGSGSGTQFTLTISDLECDDAATYYCTGTYGNSAWY
EDAFGGGTEVVVKRTPVAPTVLLFPPSSAELATGTATIVCVANKYFPDGTVTWKVDGITQSS
GINNSRTPQNSADCTYNLSSTLTLSSDEYNSHDEYTCQVAQDSGSPVVQSFSRKSC

In some embodiments, the anti-CD83 scFv V_{H} domain comprises the amino acid sequence:

(SEQ ID NO: 33, 95F04)
METGLRWLLLVAVLKGVQCQSVEESGGRLVTPGTPLTLTCTVSGIDLSSNAMIVRQAPRE
GLEWIGAMDSNSRTYYATWAKGRFTISRTSSITVDLKITSPTTEDTATYFCARGDGGSSDYT
EMWGPGTLVTVSSASTKGPSVYPLAPGSAAQTNSMVTLGCLVKGYFPEPVTVTWNSGSLS
SGVHTFPAVLQSDLYTLSSSVTVPSSTWPSETVTCNVAHPASSTKVDKKIVPRDCGCKPCI
CTVPEVSSVFIFPPKPKDVLTITLTPKVTCVVVDISKDDPEVQFSWFVDDVEVHTAQTQPRE
EQFNSTFRSVSELPIMHQDWLNGKEFKCRVNSAAFPAPIEKTISKTKGRPKAPQVYTIPPPK
EQMAKDKVSLTCMITDFFPEDITVEWQWNGQPAENYKNTQPIMDTDGSYFVYSKLNVQKS
NWEAGNTFTCSVLHEGLHNHHTEKSLSHSPGK.

In some embodiments, the anti-CD83 scFv V_{L} domain comprises the amino acid sequence:

(SEQ ID NO: 34, 95F04)
MDTRAPTQLLGLLLLWLPGATFAQAVVTQTTSPVSAPVGGTVTINCQSSQSVYGNNELSW
YQQKPGQPPKLLIYQASSLASGVPSRFKGSGSGTQFTLTISDLECDDAATYYCLGEYSISAD
NHFGGGTEVVVKRTPVAPTVLLFPPSSAELATGTATIVCVANKYFPDGTVTWKVDGITQSS
GINNSRTPQNSADCTYNLSSTLTLSSDEYNSHDEYTCQVAQDSGSPVVQSFSRKSC

In some embodiments, the anti-CD83 scFv V_{H} domain comprises the amino acid sequence:

(SEQ ID NO: 35)
QVQLVQSGGAVVQPGRSLRLSCAASGFTFSTYGMHWRQAPGKGLEWAAVSYDGSNK
YYADFVKGRFTISRDNPKNTLYLQMNSLRADDTAVYYCARRGGLDIWGQGTTVTVSSASTK
GPSVFPLAPSSKSTSGGTAALGCLVKDYFPEPVTVSWNSGALTSGVHTFPAVLQSSGLYSL
SSVTVPSSSLGTQTYICNVNHKPSNTKVDKKVEPKSCAAA.

In some embodiments, the anti-CD83 scFv V_{L} domain comprises the amino acid sequence:

(SEQ ID NO: 36)
LTQPPPASGTPGQQRVTISCSGSSSNIGSNTVNWYQQLPGTAPKLLIYY
GNDQRPSGVPDRFSASKSGTSASLAISGLQSEDEAHYYCAAWDGSLNGG
VIFGGGTKVTLG.

In some embodiments, the anti-CD83 scFv V_{L} domain comprises the amino acid sequence:

(SEQ ID NO: 37)
VTQPPSASGTPGQRVTISCSGSSSNIGTNPVNWYQQLPGTAPKLLIYTT
DQRPSGVPDRFSGSKSGTSASLAISGLQSEDEADYYCAAWDDSLSGLYV
FGTGTKVTVLG.

In some embodiments, the anti-CD83 scFv VL domain comprises the amino acid sequence:

(SEQ ID NO: 38)
MTHTPLSLSVTPGQPASISCKSSQSLLHSDGKTYLYWYLQRPGQSPQPL
IYEVSNRFSGVPDRFSGSGSGTDFTLKISRVQAEDVGVYYCMQSLQLWT
FGQGTKVEIKR.

In some embodiments, the anti-CD83 scFv V_{L} domain comprises the amino acid sequence:

(SEQ ID NO: 39)
MTQSPLSLPVTLGQPASISCRSSQSLIHSDGNTYLDWFQQRPGQSPRRL
IYKVSNRDSGVPDRFSGSGSGTDFTLRISRVEAEDIGVYYCMQATHWPR
TFGQGTKVEIKR.

In some embodiments, the anti-CD83 scFv V_{L} domain comprises the amino acid sequence:

(SEQ ID NO: 40)
MTQSPLSLPVTLGQPASISCRSSQSLVDSAGNTFLHWFHQRPGQSPRRL
IYKVSNRDSGVPDRFSGSGSGTDFTLKISRVEAEDVGVYYCMQGTHWPR
TFGQGTKVEIKR.

In some embodiments, the anti-CD83 scFv V_{L} domain comprises the amino acid sequence:

(SEQ ID NO: 41)
LTQSPLSLPVTLGQPASISCKSSQSLVDSDGNTYLNWFQQRPGQSPRRL
IYKVSNRDSGVPDRFSGSGSGTDFTLKISRVEAEDVGVYYCMQGTHWPR
TFGQGTKVEIKR.

In some embodiments, the anti-CD83 scFv V_{L} domain comprises the amino acid sequence:

(SEQ ID NO: 42)
MTQSPLSLPVTLGQPASISCRSSQSLVHSDGNMYLNWFQQRPGQSPRRL
IYKVSNRDSGVPDRFSGSGSGTDFTLKISRVEAEDVGVYYCMQATQPTW
TFGQGTKLEIKR.

In some embodiments, the anti-CD83 scFv V_{L} domain comprises the amino acid sequence:

(SEQ ID NO: 43)
MTQSPSSLSASVGDRVTITCQASQDISNYLNWYQQKPGKAPKLLIYDAS
NLETGVPSRFSGSGSGTDFTFTISSATYYCQQTYQGTKLEIKR.

In some embodiments, the anti-CD83 scFv V_{L} domain comprises the amino acid sequence:

(SEQ ID NO: 44)
MTQSPSSLSASVGHPVTITCRASQSLISYLNWYHQKPGKAPKLLIYAAS
ILQSGVPSRFSGSGSGTDFTLTISSLQPENFASYYCQHTDSFPRTFGHG
TKVEIKR.

In some embodiments, the anti-CD83 scFv V_{L} domain comprises the amino acid sequence:

(SEQ ID NO: 45)
LTQPPSASGTPGQGVTISCRGSTSNIGNNVVNWYQHVPGSAPKLLIWSN
IQRPSGIPDRFSGSKSGTSASLAISGLQSEDQAVYYCAVWDDGLAGVWF
GGGTTVTVLS.

In some embodiments, the anti-CD83 scFv V_{L} domain comprises the amino acid sequence:

(SEQ ID NO: 46)
MTQAPVVSVALEQTVRITCQGDSLAIYYDFWYQHKPGQAPVLVIYGKNN
RPSGIPHRFSGSSSNTDSLTITGAQAEDEADYYCNSRDSSGNHVWFGGG
TNLTVLG.

In some embodiments, the anti-CD83 scFv V_{L} domain comprises the amino acid sequence:

(SEQ ID NO: 47)
LTQSPLSLPVTLGQPASISCKSNQSLVHSDGNTYLNWFQQRPGQSPRRL
IYKVSNRDSGVPDRFSGSGSGTDFTLKINRVEAEDVGVYYCMQGTQWPR
TFGGQGTKLDIKR.

In some embodiments, the anti-CD83 scFv V_{H} domain has been humanized and comprises the amino acid sequence:

(SEQ ID NO: 48, VH-GBM01)
QVQLQESGPGLVKPSETLSLTCTVSGFSITTGGYWWTWIRQPPGKGLEW
IGYIFSSGNTNYNPSIKSRVTISVDTSKNQFSLKLSSVTAADTAVYYCA
RAYGKLGFDYWGQGTLVTVSS.

In some embodiments, the anti-CD83 scFv V_{H} domain has been humanized and comprises the amino acid sequence:

(SEQ ID NO: 49, VH-GBM02)
QVQLQESGPGLVKPSQTLSLTCTVSGFSITTGGYWWTWIRQHPGKGLEW
IGYIFSSGNTNYNPSIKSLVTISVDTSKNQFSLKLSSVTAADTAVYYCA
RAYGKLGFDYWGQGTLVTVSS.

In some embodiments, the anti-CD83 scFv V_{H} domain has been humanized and comprises the amino acid sequence:

(SEQ ID NO: 50, VH-GBM03)
QVQLQESGPGLVKPSQTLSLTCTVSGFSITTGGYWWTWIRQPPGKGLEW
IGYIFSSGNTNYNPSIKSRVTISVDTSKNQFSLKLSSVTAADTAVYYCA
RAYGKLGFDYWGQGTLVTVSS.

In some embodiments, the anti-CD83 scFv V_{H} domain has been humanized and comprises the amino acid sequence:

(SEQ ID NO: 51, VH-GBM04)
QVQLQESGPGLVKPSETLSLTCTVSGFSITTGGYWWTWIRQPPGKGLEW
IGYIFSSGNTNYNPSIKSRVTISRDTSKNQFSLKLSSVTAADTAVYYCA
RAYGKLGFDYWGQGTLVTVSS.

In some embodiments, the anti-CD83 scFv V_{H} domain has been humanized and comprises the amino acid sequence:

(SEQ ID NO: 52, VH-GBM05)
QVQLQESGPGLVKPSETLSLTCTVSGFSITTGGYWWTWIRQPPGKGLEW
IGYIFSSGNTNYNPSIKSRVTISVDTSKNQFSLKLSSVTAADTARYYCA
RAYGKLGFDYWGQGTLVTVSS.

In some embodiments, the anti-CD83 scFv V_{H} domain has been humanized and comprises the amino acid sequence:

(SEQ ID NO: 53, VH-GBM06)
QVQLQESGPGLVKPSETLSLTCTVSGFSITTGGYWWTWIRQPPGKGLEW
IGYIFSSGNTNYNPSIKSRISITRDTSKNQFFLQLNSVTTEGDTARYYC
ARAYGKLGFDYWGQGTLVTVSS.

In some embodiments, the anti-CD83 scFv V_{L} domain has been humanized and comprises the amino acid sequence:

(SEQ ID NO: 54, VL-GBM01)
QLVLTQSPSASASLGASVKLTCTLSSQHSTYTIGWHQQQPEKGPRYLMK
VNSDGSHSKGDGIPDRFSGSSSGAERYLTISSLQSEDEADYYCGSSDSS
GYVFGSGTKVTVL.

In some embodiments, the anti-CD83 scFv V_{L} domain has been humanized and comprises the amino acid sequence:

(SEQ ID NO: 55, VL-GBM02)
LPVLTQPPSASALLGASIKLTCTLSSQHSTYTIGWYQQRPGRSPQYIMK
VNSDGSHSKGDGIPDRFMGSSSGADRYLTFSNLQSDDEAEYHCGSSDSS
GYVFGSGTKVTVL.

The heavy and light chains are preferably separated by a linker. Suitable linkers for scFv antibodies are known in the art. In some embodiments, the linker comprises the amino acid sequence GGGGSGGGGSGGGGS (SEQ ID NO:56).

In some embodiments, the anti-CD83 scFv comprises an amino acid sequence:

(SEQ ID NO: 57)
QPVLTQSPSASASLGNSVKITCTLSSQHSTYTIGWYQQHPDKAPKYVMY
VNSDGSHSKGDGIPDRFSGSSSGAHRYLSISNIQPEDEADYFCGSSDSS
GYVFGSGTQLTVLRAAASSGGGGSGGGGSGGGGSQPVLTQSPSASASLG
NSVKITCTLSSQHSTYTIGWYQQHPDKAPKYVMYVNSDGSHSKGDGIPD
RFSGSSSGAHRYLSISNIQPEDEADYFCGSSDSSGYVFGSGTQLTVLRA
AA.

In some embodiments, the anti-CD83 scFv comprises an amino acid sequence:

(SEQ ID NO: 58)
QVQLKESGPGLVKPSQSLSLTCSVTGFSITTGGYWWTWIRQFPGQKLEW
MGYIFSSGNTNYNPSIKSRISITRDTSKNQFFLQLNSVTTEGDTARYYC
ARAYGKLGFDYWGQGTLVTVSSGGGGSGGGGSGGGGSQVQLKESGPGLV
KPSQSLSLTCSVTGFSITTGGYWWTWIRQFPGQKLEWMGYIFSSGNTNY
NPSIKSRISITRDTSKNQFFLQLNSVTTEGDTARYYCARAYGKLGFDYW
GQGTLVTV.

In some embodiments, the anti-CD83 scFv comprises an amino acid sequence:

(SEQ ID NO: 59)
QVQLQESGPGLVKPSETLSLTCTVSGFSITTGGYW
WTWIRQPPGKGLEWIGYIFSSGNTNYNPSIKSRVT
ISVDTSKNQFSLKLSSVTAADTAVYYCARAYGKLG
FDYWGQGTLVTVSSGGGGSGGGGSGGGGSQLVLTQ
SPSASASLGASVKLTCTLSSQHSTYTIGWHQQQPE
KGPRYLMKVNSDGSHSKGDGIPDRFSGSSSGAERY
LTISSLQSEDEADYYCGSSDSSGYVFGSGTKVTVL.

In some embodiments, the anti-CD83 scFv comprises an amino acid sequence:

(SEQ ID NO: 60
QVQLQESGPGLVKPSQTLSLTCTVSGFSITTGGYW
WTWIRQHPGKGLEWIGYIFSSGNTNYNPSIKSLVT
ISVDTSKNQFSLKLSSVTAADTAVYYCARAYGKLG
FDYWGQGTLVTVSSGGGGSGGGGSGGGGSQLVLTQ
SPSASASLGASVKLTCTLSSQHSTYTIGWHQQQPE
KGPRYLMKVNSDGSHSKGDGIPDRFSGSSSGAERY
LTISSLQSEDEADYYCGSSDSSGYVFGSGTKVTVL.

In some embodiments, the anti-CD83 scFv comprises an amino acid sequence:

(SEQ ID NO: 61)
QVQLQESGPGLVKPSQTLSLTCTVSGFSITTGGYW
WTWIRQPPGKGLEWIGYIFSSGNTNYNPSIKSRVT
ISVDTSKNQFSLKLSSVTAADTAVYYCARAYGKLG
FDYWGQGTLVTVSSGGGGSGGGGSGGGGSQLVLTQ
SPSASASLGASVKLTCTLSSQHSTYTIGWHQQQPE
KGPRYLMKVNSDGSHSKGDGIPDRFSGSSSGAERY
LTISSLQSEDEADYYCGSSDSSGYVFGSGTKVTVL.

In some embodiments, the anti-CD83 scFv comprises an amino acid sequence:

(SEQ ID NO: 62)
QVQLQESGPGLVKPSETLSLTCTVSGFSITTGGYW
WTWIRQPPGKGLEWIGYIFSSGNTNYNPSIKSRVT
ISRDTSKNQFSLKLSSVTAADTAVYYCARAYGKLG
FDYWGQGTLVTVSSGGGGSGGGGSGGGGSQLVLTQ
SPSASASLGASVKLTCTLSSQHSTYTIGWHQQQPE
KGPRYLMKVNSDGSHSKGDGIPDRFSGSSSGAERY
LTISSLQSEDEADYYCGSSDSSGYVFGSGTKVTVL.

In some embodiments, the anti-CD83 scFv comprises an amino acid sequence:

(SEQ ID NO: 63)
QVQLQESGPGLVKPSETLSLTCTVSGFSITTGGYW
WTWIRQPPGKGLEWIGYIFSSGNTNYNPSIKSRVT
ISVDTSKNQFSLKLSSVTAADTARYYCARAYGKLG
FDYWGQGTLVTVSSGGGGSGGGGSGGGGSQLVLTQ
SPSASASLGASVKLTCTLSSQHSTYTIGWHQQQPE
KGPRYLMKVNSDGSHSKGDGIPDRFSGSSSGAERY
LTISSLQSEDEADYYCGSSDSSGYVFGSGTKVTVL.

In some embodiments, the anti-CD83 scFv comprises an amino acid sequence:

(SEQ ID NO: 64)
QVQLQESGPGLVKPSETLSLTCTVSGFSITTGGYW
WTWIRQPPGKGLEWIGYIFSSGNTNYNPSIKSRIS
ITRDTSKNQFFLQLNSVTTEGDTARYYCARAYGKL
GFDYWGQGTLVTVSSGGGGSGGGGSGGGGSQLVLT
QSPSASASLGASVKLTCTLSSQHSTYTIGWHQQQP
EKGPRYLMKVNSDGSHSKGDGIPDRFSGSSSGAER
YLTISSLQSEDEADYYCGSSDSSGYVFGSGTKVTV
L.

In some embodiments, the anti-CD83 scFv comprises an amino acid sequence:

(SEQ ID NO: 65)
QVQLQESGPGLVKPSETLSLTCTVSGFSITTGGYW
WTWIRQPPGKGLEWIGYIFSSGNTNYNPSIKSRVT
ISVDTSKNQFSLKLSSVTAADTAVYYCARAYGKLG
FDYWGQGTLVTVSSGGGGSGGGGSGGGGSLPVLTQ
PPSASALLGASIKLTCTLSSQHSTYTIGWYQQRPG
RSPQYIMKVNSDGSHSKGDGIPDRFMGSSSGADRY
LTFSNLQSDDEAEYHCGSSDSSGYVFGSGTKVTVL.

In some embodiments, the anti-CD83 scFv comprises an amino acid sequence:

(SEQ ID NO: 66)
QVQLQESGPGLVKPSQTLSLTCTVSGFSITTGGYW
WTWIRQHPGKGLEWIGYIFSSGNTNYNPSIKSLVT
ISVDTSKNQFSLKLSSVTAADTAVYYCARAYGKLG
FDYWGQGTLVTVSSGGGGSGGGGSGGGGSLPVLTQ
PPSASALLGASIKLTCTLSSQHSTYTIGWYQQRPG
RSPQYIMKVNSDGSHSKGDGIPDRFMGSSSGADRY
LTFSNLQSDDEAEYHCGSSDSSGYVFGSGTKVTVL.

In some embodiments, the anti-CD83 scFv comprises an amino acid sequence:

(SEQ ID NO: 67)
QVQLQESGPGLVKPSQTLSLTCTVSGFSITTGGYW
WTWIRQPPGKGLEWIGYIFSSGNTNYNPSIKSRVT
ISVDTSKNQFSLKLSSVTAADTAVYYCARAYGKLG
FDYWGQGTLVTVSSGGGGSGGGGSGGGGSLPVLTQ
PPSASALLGASIKLTCTLSSQHSTYTIGWYQQRPG
RSPQYIMKVNSDGSHSKGDGIPDRFMGSSSGADRY
LTFSNLQSDDEAEYHCGSSDSSGYVFGSGTKVTVL.

In some embodiments, the anti-CD83 scFv comprises an amino acid sequence:

(SEQ ID NO: 68)
QVQLQESGPGLVKPSETLSLTCTVSGFSITTGGYW
WTWIRQPPGKGLEWIGYIFSSGNTNYNPSIKSRVT
ISRDTSKNQFSLKLSSVTAADTAVYYCARAYGKLG
FDYWGQGTLVTVSSGGGGSGGGGSGGGGSLPVLTQ
PPSASALLGASIKLTCTLSSQHSTYTIGWYQQRPG
RSPQYIMKVNSDGSHSKGDGIPDRFMGSSSGADRY
LTFSNLQSDDEAEYHCGSSDSSGYVFGSGTKVTVL.

In some embodiments, the anti-CD83 scFv comprises an amino acid sequence:

(SEQ ID NO: 69)
QVQLQESGPGLVKPSETLSLTCTVSGFSITTGGYW
WTWIRQPPGKGLEWIGYIFSSGNTNYNPSIKSRVT
ISVDTSKNQFSLKLSSVTAADTARYYCARAYGKLG
FDYWGQGTLVTVSSGGGGSGGGGSGGGGSLPVLTQ
PPSASALLGASIKLTCTLSSQHSTYTIGWYQQRPG
RSPQYIMKVNSDGSHSKGDGIPDRFMGSSSGADRY
LTFSNLQSDDEAEYHCGSSDSSGYVFGSGTKVTVL.

In some embodiments, the anti-CD83 scFv comprises an amino acid sequence:

(SEQ ID NO: 70)
QVQLQESGPGLVKPSETLSLTCTVSGFSITTGGYW
WTWIRQPPGKGLEWIGYIFSSGNTNYNPSIKSRIS
ITRDTSKNQFFLQLNSVTTEGDTARYYCARAYGKL
GFDYWGQGTLVTVSSGGGGSGGGGSGGGGSLPVLT
QPPSASALLGASIKLTCTLSSQHSTYTIGWYQQRP
GRSPQYIMKVNSDGSHSKGDGIPDRFMGSSSGADR
YLTFSNLQSDDEAEYHCGSSDSSGYVFGSGTKVTV
L.

In some embodiments, the anti-CD83 scFv comprises an amino acid sequence:

(SEQ ID NO: 71)
QVQLKESGPGLVKPSQSLSLTCSVTGFSITTGGYW
WTWIRQFPGQKLEWMGYIFSSGNTNYNPSIKSRIS
ITRDTSKNQFFLQLNSVTTEGDTARYYCARAYGKL
GFDYWGQGTLVTVSSGGGGSGGGGSGGGGSQPVLT
QSPSASASLGNSVKITCTLSSQHSTYTIGWYQQHP
DKAPKYVMYVNSDGSHSKGDGIPDRFSGSSSGAHR
YLSISNIQPEDEADYFCGSSDSSGYVFGSGTQLTV
L.

As with other CARs, the disclosed polypeptides can also contain a transmembrane domain and an endodomain capable of activating an immune effector cell. For example, the endodomain can contain a signaling domain and one or more co-stimulatory signaling regions.

In some embodiments, the intracellular signaling domain is a CD3 zeta (CD3ζ) signaling domain. In some embodiments, the costimulatory signaling region comprises the cytoplasmic domain of CD28, 4-1BB, or a combination thereof. In some cases, the costimulatory signaling region contains 1, 2, 3, or 4 cytoplasmic domains of one or more intracellular signaling and/or costimulatory molecules. In some embodiments, the co-stimulatory signaling region contains one or more mutations in the cytoplasmic domains of CD28 and/or 4-1BB that enhance signaling.

In some embodiments, the CAR polypeptide contains an incomplete endodomain. For example, the CAR polypeptide can contain only an intracellular signaling domain or a co-stimulatory domain, but not both. In these embodiments, the immune effector cell is not activated unless it and a second CAR polypeptide (or endogenous T-cell receptor) that contains the missing domain both bind their respective antigens. Therefore, in some embodiments, the CAR polypeptide contains a CD3 zeta (CD3ζ) signaling domain but does not contain a costimulatory signaling region (CSR). In other embodiments, the CAR polypeptide contains the cytoplasmic domain of CD28, 4-1BB, or a combination thereof, but does not contain a CD3 zeta (CD3ζ) signaling domain (SD).

Also disclosed are isolated nucleic acid sequences encoding the disclosed CAR polypeptides, vectors comprising these isolated nucleic acids, and regulatory T cells containing these vectors. In some embodiments, the cell suppresses alloreactive donor cells, such as T cells, when the antigen binding domain of the CAR binds to CD83.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1: AAPC can be engineered to express CD3, CD28, and CD137L. Human AAPC cell line was transduced with gamma retro virus to express CD3 scFv(GFP), CD28 scFv(mcherry), and CD137L. A) Schematic of CD3 and CD28 scFv tagged with fluorescent reporters and CD137L. B/Flow contour plots of AAPC expressing CD3 and CD28 positive for GFP and mcherry respectively and CD137L stained with PE.

FIG. 2: AAPC efficiently expand donor human T cells. T cells isolated from peripheral blood mononuclear cells of healthy donor were stimulated with various AAPCs and beads for 2 weeks in G-REX. A) CD3 T fold expansion. B) CD8 T central memory cells fold expansion. C) CD4 T cell fold expansion on day 10 at various AAPC to T cell ratio. D) CD4 T central memory cell fold expansion.

FIG. 3: T cells expanded with AAPC had comparable proliferation compared to Beads CD3/28. Human T cells were isolated from PBMCs stained with CFSE dye and expanded with either AAPC CD3/28 or Beads CD3/28 for 6 days. Left plots are proliferation of CD4 T cells and right plots are proliferation of CD8 T cells.

FIG. 4: AAPC can be used to aid in transduction of gamma retrovirus instead of retronectin. A) AAPC was engineered to express HBD with Cerulean reporter. B) Human T cells were isolated from PBMCs and expanded with either AAPC CD3/28/HBD or Beads CD3/28 and retronectin for 6 days. T cells were isolated and T cells were analyzed via flow for CAR T transduction.

FIG. 5: T cells expanded with AAPC showed lower exhaustion compared to Beads CD3/28. Human T cells were isolated from PBMCs and expanded with either AAPC CD3/28 or Beads CD3/28 for 14 days. A) Flow plot of PD-1. B) Flow plot of CTLA-4.

FIG. 6: Large scale expansion of T cells using AAPC and G-REX. Human T cells were isolated and expanded using AAPC and G-REX for 14 days. Cells were also transduced with anti CD33 CAR. A) T Cells were analyzed via flow for CD8 memory subtypes. B) Total T cells were counted and plotted on log scale. C) CD33 targeted CAR T cells expanded with either AAPC or Beads were used to see their cytotoxicity against CHO CD33 cells. Untransduced T cells were used as a control.

FIG. 7 shows CD3 in live cells at day 0, 9, and 14 with aAPC:Treg at 0:1, 5:1, 10:1, 50:1, 100:1, and 150:1.

FIGS. 8A to 8C show CD3 percentage (FIG. 8A), number (FIG. 8B), and fold change (FIG. 8C) in live cells at day 0, 9, and 14 with aAPC:Treg at 0:1, 5:1, 10:1, 50:1, 100:1, and 150:1

FIG. 9 shows CD4+/CD3+ cells at day 0, 9, and 14 with aAPC:Treg at 0:1, 5:1, 10:1, 50:1, 100:1, and 150:1.

FIGS. 10A to 10C show CD4+ percentage in CD3+ T cells (FIG. 10A), CD4+/CD3+ cell number (FIG. 10B), and CD4+/CD3+ fold change (FIG. 10C) at day 0, 9, and 14 with aAPC:Treg at 0:1, 5:1, 10:1, 50:1, 100:1, and 150:1.

FIG. 11 shows CD25+/CD4+ cells at day 0, 9, and 14 with aAPC:Treg at 0:1, 5:1, 10:1, 50:1, 100:1, and 150:1.

FIGS. 12A to 12C show CD25+CD4+ percentage in CD3+ T cells (FIG. 12A), CD25+CD4+/CD3+ cell number (FIG. 12B), and CD25+CD4+/CD3+ fold change (FIG. 12C) at day 0, 9, and 14 with aAPC:Treg at 0:1, 5:1, 10:1, 50:1, 100:1, and 150:1.

FIG. 13 shows CD25+CD4+CD127lowFoxP3/CD3+ cells at day 0, 9, and 14 with aAPC:Treg at 0:1, 5:1, 10:1, 50:1, 100:1, and 150:1.

FIGS. 14A to 14C show CD25+CD4+CD127lowFoxP3 percentage in CD3+ T cells (FIG. 14A), CD25+CD4+CD127lowFoxP3/CD3+ cell number (FIG. 14B), and CD25+CD4+CD127lowFoxP3/CD3+ fold change (FIG. 14C) at day 0, 9, and 14 with aAPC:Treg at 0:1, 5:1, 10:1, 50:1, 100:1, and 150:1.

FIG. 15 shows CD28+ in CD127lowFoxP3+ cells at day 0, 9, and 14 with aAPC:Treg at 0:1, 5:1, 10:1, 50:1, 100:1, and 150:1.

FIGS. 16A to 16C show CD28+ percentage in CD127lowFoxP3+ T cells (FIG. 16A), CD28+/CD127lowFoxP3+ cell number (FIG. 16B), and CD28+/CD127lowFoxP3+ fold change (FIG. 16C) at day 0, 9, and 14 with aAPC:Treg at 0:1, 5:1, 10:1, 50:1, 100:1, and 150:1.

FIG. 17 shows CD69+/CD127lowFoxP3+ cells at day 0, 9, and 14 with aAPC:Treg at 0:1, 5:1, 10:1, 50:1, 100:1, and 150:1.

FIGS. 18A to 18C show CD69+ percentage in CD127lowFoxP3+ T cells (FIG. 18A), CD69+/CD127lowFoxP3+ cell number (FIG. 18B), and CD69+/CD127lowFoxP3+ fold change (FIG. 18C) at day 0, 9, and 14 with aAPC:Treg at 0:1, 5:1, 10:1, 50:1, 100:1, and 150:1.

FIG. 19 shows CTLA4+/CD127lowFoxP3+ cells at day 0, 9, and 14 with aAPC:Treg at 0:1, 5:1, 10:1, 50:1, 100:1, and 150:1.

FIGS. 20A to 20C show CTLA4+ percentage in CD127lowFoxP3+ T cells (FIG. 20A), CTLA4+/CD127lowFoxP3+ cell number (FIG. 20B), and CTLA4+/CD127lowFoxP3+ fold change (FIG. 20C) at day 0, 9, and 14 with aAPC:Treg at 0:1, 5:1, 10:1, 50:1, 100:1, and 150:1.

FIG. 21 shows LAG3+/CD127lowFoxP3+ cells at day 0, 9, and 14 with aAPC:Treg at 0:1, 5:1, 10:1, 50:1, 100:1, and 150:1.

FIGS. 22A to 22C show LAG3+ percentage in CD127lowFoxP3+ T cells (FIG. 22A), LAG3+/CD127lowFoxP3+ cell number (FIG. 22B), and LAG3+/CD127lowFoxP3+ fold change (FIG. 22C) at day 0, 9, and 14 with aAPC:Treg at 0:1, 5:1, 10:1, 50:1, 100:1, and 150:1.

FIG. 23 shows PD1+/CD127lowFoxP3+ cells at day 0, 9, and 14 with aAPC:Treg at 0:1, 5:1, 10:1, 50:1, 100:1, and 150:1.

FIGS. 24A to 24C show PD1+ percentage in CD127lowFoxP3+ T cells (FIG. 24A), PD1+/CD127lowFoxP3+ cell number (FIG. 24B), and PD1+/CD127lowFoxP3+ fold change (FIG. 24C) at day 0, 9, and 14 with aAPC:Treg at 0:1, 5:1, 10:1, 50:1, 100:1, and 150:1.

FIG. 25 shows TIM3+/CD127lowFoxP3+ cells at day 0, 9, and 14 with aAPC:Treg at 0:1, 5:1, 10:1, 50:1, 100:1, and 150:1.

FIGS. 26A to 26C show TIM3+ percentage in CD127lowFoxP3+ T cells (FIG. 26A), TIM3+/CD127lowFoxP3+ cell number (FIG. 26B), and TIM3+/CD127lowFoxP3+ fold change (FIG. 26C) at day 0, 9, and 14 with aAPC:Treg at 0:1, 5:1, 10:1, 50:1, 100:1, and 150:1.

FIGS. 27A to 27C show CD3 percentage (FIG. 27A), number (FIG. 278B), and fold change (FIG. 27C) in live cells at day 0, 11, and 14.

FIGS. 28A to 28C show CD4+ percentage in CD3+ T cells (FIG. 28A), CD4+/CD3+ cell number (FIG. 28B), and CD4+/CD3+ fold change (FIG. 28C) at day 0, 11, and 14.

FIGS. 29A to 29C show CD25+CD4+ percentage in CD3+ T cells (FIG. 29A), CD25+CD4+/CD3+ cell number (FIG. 29B), and CD25+CD4+/CD3+ fold change (FIG. 29C) at day 0, 11, and 14.

FIGS. 30A to 30C show CD25+CD4+CD127lowFoxP3 percentage in CD3+ T cells (FIG. 30A), CD25+CD4+CD127lowFoxP3/CD3+ cell number (FIG. 30B), and CD25+CD4+CD127lowFoxP3/CD3+ fold change (FIG. 30C) at day 0, 11, and 14.

FIGS. 31A to 31C show CD28+ percentage in CD127lowFoxP3+ T cells (FIG. 31A), CD28+CD127lowFoxP3+ cell number (FIG. 31B), and CD28+CD127lowFoxP3+ fold change (FIG. 31C) at day 0, 11, and 14.

FIGS. 32A to 32C show CD69+ percentage in CD127lowFoxP3+ T cells (FIG. 32A), CD28+CD127lowFoxP3+ cell number (FIG. 32B), and CD28+CD127lowFoxP3+ fold change (FIG. 32C) at day 0, 11, and 14.

FIGS. 33A to 33C show CTLA4+ percentage in CD127lowFoxP3+ T cells (FIG. 33A), CTLA4+CD127lowFoxP3+ cell number (FIG. 33B), and CTLA4+CD127lowFoxP3+ fold change (FIG. 33C) at day 0, 11, and 14.

FIGS. 34A to 34C show LAG3+ percentage in CD127lowFoxP3+ T cells (FIG. 34A), LAG3+CD127lowFoxP3+ cell number (FIG. 34B), and LAG3+CD127lowFoxP3+ fold change (FIG. 34C) at day 0, 11, and 14.

FIGS. 35A to 35C show PD1+ percentage in CD127lowFoxP3+ T cells (FIG. 35A), PD1+CD127lowFoxP3+ cell number (FIG. 35B), and PD1+CD127lowFoxP3+ fold change (FIG. 35C) at day 0, 11, and 14.

FIGS. 36A to 36C show TIM3+ percentage in CD127lowFoxP3+ T cells (FIG. 36A), TIM3+CD127lowFoxP3+ cell number (FIG. 6B), and TIM3+CD127lowFoxP3+ fold change (FIG. 36C) at day 0, 11, and 14.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “antibody” refers to natural or synthetic antibodies that selectively bind a target antigen. The term includes polyclonal and monoclonal antibodies. In addition to intact immunoglobulin molecules, also included in the term “antibodies” are fragments or polymers of those immunoglobulin molecules, and human or humanized versions of immunoglobulin molecules that selectively bind the target antigen.

As used herein, the term “antibody or fragments thereof” encompasses chimeric antibodies and hybrid antibodies, with dual or multiple antigen or epitope specificities, and fragments, such as F(ab′)2, Fab′, Fab, scFv, and the like, including hybrid fragments. Thus, fragments of the antibodies that retain the ability to bind their specific antigens are provided. For example, fragments of antibodies which maintain CD3, CD28, CD137, PD1, CTLA4, LAG3, TIM3, BTLA, CD160, 2B4, A2aR, and KIR binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)). Also included within the meaning of “antibody or fragments thereof” are conjugates of antibody fragments and antigen binding proteins (single chain antibodies). The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992). As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

The term “specifically binds”, as used herein, when referring to a polypeptide (including antibodies) or receptor, refers to a binding reaction which is determinative of the presence of the protein or polypeptide or receptor in a heterogeneous population of proteins and other biologics. Thus, under designated conditions (e g immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 105 M-1 (e.g., 106 M-1, 107 M-1, 108 M-1, 109 M-1, 1010 M-1, 1011 M-1, and 1012 M-1 or more) with that second molecule.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

### Methods for Producing Regulatory CAR T Cells

Regulatory T cells (T_{reg} cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Disclosed herein are methods for producing efficacious CAR T_{reg} cells in a GMP-scalable system.

AAPCs that Activate T_{reg} Cells

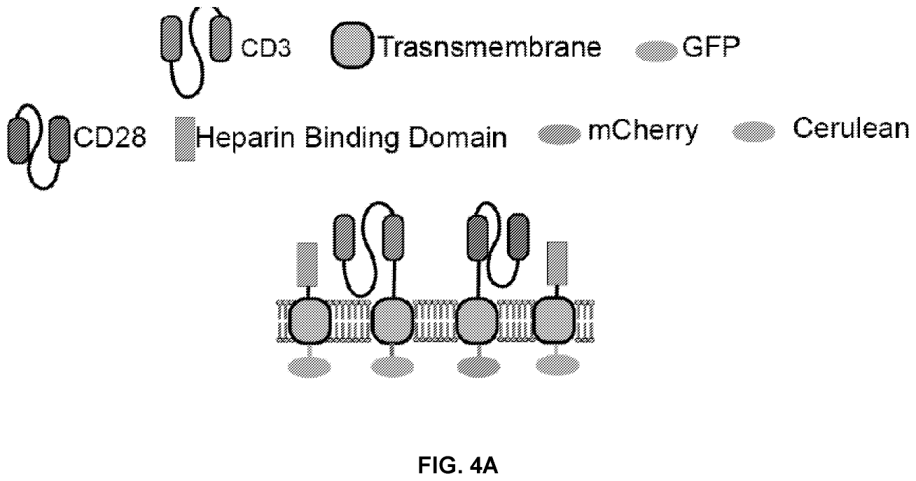

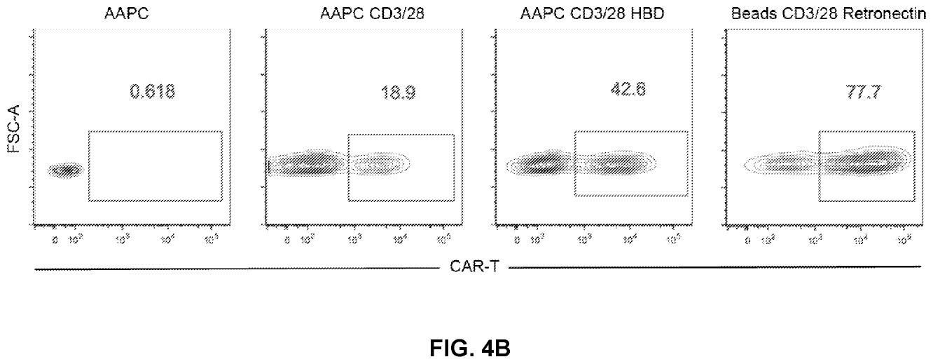

Disclosed herein are methods for producing efficacious CAR T_{reg} cells in a GMP-scalable system that involves the use of artificial antigen presenting cells (AAPC) engineered to express anti-CD3, anti-CD28, and/or CD137 (41BBL) that activate and expand T_{reg} cells. In some embodiments, the AAPCs are further modified to express the IL15 receptor (IL15R), which provide IL15 co-stimulation, thereby promoting T_{reg} expansion. In some embodiments, the AAPC is modified to express the heparin-binding domain (HBD), which binds retrovirus, to facilitate retroviral gene transfer of the CAR T_{reg} cells (FIG. 4). The AAPC that ligate CD3 and bind virus via HBD support efficient gene transfer of primary human T cells (FIG. 4).

Methods for producing AAPCs are described for example in US 2019/0262400 and US 2019/0262400, which are incorporated by reference in their entireties for the teaching of how to make and use AAPCs. The aAPC can be derived from any antigen presenting cell including a cell line such as, for example K562, NIH/3T3, Chinese hamster ovary (CHO), or Human Embryonic Kidney (HEK) cell line.

It is understood and herein contemplated that the disclosed methods can result in an expanded immune cell. Accordingly, in one aspect disclosed herein are immune cells produced by any method for expanding immune cells disclosed herein.

In some embodiments, the nucleic acid sequence for anti-CD3 scFv is:

(SEQ ID NO: 72)
ATGGCCAGCCCCCTGACAAGATTCCTGAGCCTGAA
CCTGCTGCTGCTGGGCGAGTCCATCATCCTCGGCA
GCGGCGAGGCCCAAGTGCAACTGCAGGAATCCGGA
GCTGAGCTGGCCAGACCCGGAGCCAGCGTGAAGAT
GTCCTGTAAGGCCTCCGGCTACACCTTCACCAGGT
ACACCATGCACTGGGTGAAGCAGAGGCCTGGCCAG
GGCCTGGAGTGGATCGGCTACATCAATCCCAGCAG
GGGCTACACCAATTACAATCAGAAGTTCAAGGACA
AGGCCACCCTCACAACCGATAAGAGCAGCTCCACC
GCCTACATGCAACTGAGCAGCCTGACCTCCGAGGA
CTCCGCCGTGTACTACTGTGCCAGATACTACGACG
ACCACTACTGCCTGGACTACTGGGGCCAGGGCACC
ACAGTGACAGTGAGCTCCGGCGGCGGCGGCTCCGG
CGGCGGCGGCAGCGGCGGCGGCGGAAGCGACATTC
AGCTGACCCAGAGCCCCGCTATCATGTCCGCCAGC
CCTGGAGAGAAGGTGACCATGACCTGCAGCGCCAG
CAGCAGCGTGAGCTACATGAACTGGTACCAGCAGA
AGTCCGGCACCAGCCCCAAGAGGTGGATCTACGAC
ACCAGCAAGCTGGCTTCCGGCGTGCCTGCCCACTT
TAGAGGCAGCGGCTCCGGCACCAGCTACAGCCTGA
CCATCAGCGGCATGGAGGCTGAAGACGCCGCCACC
TACTACTGTCAGCAGTGGTCCTCCAACCCCTTCAC
CTTCGGCAGCGGCACAAAGCTGGAGATCAAGAGA.

In some embodiments, the amino acid sequence for anti-CD3 scFv is:

(SEQ ID NO: 73)
MASPLTRFLSLNLLLLGESIILGSGEAQVQLQESGAELARPGASVKMSCKASGYTFTRYTMH
WWKQRPGQGLEWIGYINPSRGYTNYNQKFKDKATLTTDKSSSTAYMQLSSLTSEDSAVYY
CARYYDDHYCLDYWGQGTTVTVSSGGGGSGGGGSGGGGSDIQLTQSPAIMSASPGEKVT
MTCSASSSVSYMNWYQQKSGTSPKRWIYDTSKLASGVPAHFRGSGSGTSYSLTISGMEAE
DAATYYCOQWSSNPFTFGSGTKLEIKR.

In some embodiments, the nucleic acid sequence for anti-CD28 is:

(SEQ ID NO: 74)
ATGGCCTCCCCCCTCACCAGGTTCCTGAGCCTGAACCTGCTGCTGCTGGGCGAGAGC
ATCATCCTGGGAAGCGGCGAGGCTCAGGTGCAGCTGCAGCAGAGCGGCACCGAACTG
GTGAAGCCTGCCTCCAGCGTGAAGATCAGCTGCAAGGCCAGCGGCTACACCTCCACC
AGCAACTACATGCACTGGATCAGGCAGCAGCCCGGCAATGGCCTGGAGTGGATCGGC
AGGATTTACCCCGGCAACGGCAACACCAAATACAATCAGAAGTTCGATGGCAAGGCCA
CCCCCACCGCTGACAAGTCCTCCTCCACCGCCTACATGCAGCTGAGCAGACTGACCTT
CGAGGATAGCGCCGTCTACTTCTGCGCTAGCGCCCCTCTGGACTACGGAGGCCACATC
ATGGACGCCTGGGGCCAGGGCACCACAGTGACAGTGAGCTCCGGAGGCGGCGGCAG
CGGCGGCGGCGGCAGCGGCGGAGGCGGCAGCGATATCCAGCTCACCCAGAGCCCTG
CCTTTCTGAGCGCCAGCCTGGGCGAGACCGTGAGCATTGAGTGCCTCGGCTCCGAGG
ACATCTACGGCTACCTGGCCTGGTATCAGCAGAAGCCCGGCAAGAGCCCCCAGCTGCT
GATCTACGTGGCCAACAGGCTGCAGGATGGCGTGCCTAGCAGATTTAGCGGCAGCGG
CAGCGGAACCCAGTATTCCCTCAAGATCAGCGGCATGCAGCCTGAGGACGAGGGCGA
CTACTACTGCCTCCAGGGCAGCAAGTTTCCCCTCACCTTCGGCAGCGGCACCAAGCTG
GAGATCAAA.

In some embodiments, the amino acid sequence for anti-CD28 is:

(SEQ ID NO: 75)
MASPLTRFLSLNLLLLGESIILGSGEAQVQLQQSGTELVKPASSVKISCKASGYTSTSNYMH
WIRQQPGNGLEWIGRIYPGNGNTKYNQKFDGKATPTADKSSSTAYMQLSRLTFEDSAVYF
CASAPLDYGGHIMDAWGQGTTVTVSSGGGGSGGGGSGGGGSDIQLTQSPAFLSASLGET
VSIECLGSEDIYGYLAWYQQKPGKSPQLLIYVANRLQDGVPSRFSGSGSGTQYSLKISGMQ
PEDEGDYYCLQGSKFPLTFGSGTKLEIK.

In some embodiments, the nucleic acid sequence for 41 BBL is:

(SEQ ID NO: 76)
ATGGAGTACGCCAGCGACGCCAGCCTGGACCCCGAGGCCCCCTGGCCCCCCGCCCC
CCGGGCCCGGGCCTGCCGGGTGCTGCCCTGGGCCCTGGTGGCCGGCCTGCTGCTGC
TGCTGCTGCTGGCCGCCGCCTGCGCCGTGTTCCTGGCCTGCCCCTGGGCCGTGAGCG
GCGCCCGGGCCAGCCCCGGCAGCGCCGCCAGCCCCCGGCTGCGGGAGGGCCCCGA
GCTGAGCCCCGACGACCCCGCCGGCCTGCTGGACCTGCGGCAGGGCATGTTCGCCC
AGCTGGTGGCCCAGAACGTGCTGCTGATCGACGGCCCCCTGAGCTGGTACAGCGACC
CCGGCCTGGCCGGCGTGAGCCTGACCGGCGGCCTGAGCTACAAGGAGGACACCAAG
GAGCTGGTGGTGGCCAAGGCCGGCGTGTACTACGTGTTCTTCCAGCTGGAGCTGCGG
CGGGTGGTGGCCGGCGAGGGCAGCGGCAGCGTGAGCCTGGCCCTGCACCTGCAGCC
CCTGCGGAGCGCCGCCGGCGCCGCCGCCCTGGCCCTGACCGTGGACCTGCCCCCCG
CCAGCAGCGAGGCCCGGAACAGCGCCTTCGGCTTCCAGGGCCGGCTGCTGCACCTGA
GCGCCGGCCAGCGGCTGGGCGTGCACCTGCACACCGAGGCCCGGGCCCGGCACGCC
TGGCAGCTGACCCAGGGCGCCACCGTGCTGGGCCTGTTCCGGGTGACCCCCGAGATC
CCCGCCGGCCTGCCCAGCCCCCGGAGCGAG.

In some embodiments, the amino acid sequence for 41 BBL is:

(SEQ ID NO: 77)
MEYASDASLDPEAPWPPAPRARACRVLPWALVAGLLLLLLLAAACAVFLACPWAVSGARA
SPGSAASPRLREGPELSPDDPAGLLDLRQGMFAQLVAQNVLLIDGPLSWYSDPGLAGVSLT
GGLSYKEDTKELVVAKAGVYYVFFQLELRRVVAGEGSGSVSLALHLQPLRSAAGAAALALT
VDLPPASSEARNSAFGFQGRLLHLSAGQRLGVHLHTEARARHAWQLTQGATVLGLFRVTP
EIPAGLPSPRSE.

In some embodiments, the nucleic acid sequence for IL15R is:

(SEQ ID NO: 78)
ATGGCCCCCAGAAGAGCCAGAGGCTGCAGAACCCTCGGACTGCCCGCTTTACTGCTG
CTGTTACTGCTGAGACCCCCCGCTACCAGAGGCATTACTTGTCCCCCTCCCATGAGCG
TCGAGCATGCCGACATCTGGGTGAAGAGCTACTCTTTATATTCTCGTGAGAGGTACATC
TGCAACTCCGGCTTCAAGAGAAAGGCCGGCACAAGCTCTTTAACCGAGTGCGTGCTGA
ACAAGGCTACAAACGTGGCCCACTGGACCACCCCCTCTTTAAAATGCATTCGTGATCCC
GCTCTGGTGCACCAGAGACCCGCTCCTCCCTCCACCGTGACCACAGCCGGAGTGACC
CCTCAGCCCGAATCCCTCTCCCCCTCCGGAAAGGAGCCCGCTGCCTCCAGCCCCTCC
AGCAACAACACCGCTGCCACAACCGCCGCTATCGTGCCCGGTTCCCAGCTGATGCCCT
CCAAGAGCCCCAGCACCGGAACCACCGAGATCAGCAGCCACGAGAGCTCCCACGGAA
CACCCAGCCAGACCACCGCCAAGAATTGGGAACTGACCGCCTCCGCCAGCCATCAGC
CTCCCGGCGTGTATCCTCAAGGTCACAGCGACACCACAGTGGCCATTTCCACCAGCAC
CGTGTTATTATGCGGACTGTCCGCTGTGTCTTTACTGGCTTGTTATTTAAAGTCTCGTCA
GACCCCTCCTCTGGCTTCCGTGGAGATGGAGGCCATGGAGGCTTTACCCGTGACTTGG
GGAACCTCCTCTCGTGACGAGGATTTAGAGAACTGCAGCCATCATTTATGA.

In some embodiments, the amino acid sequence for 41 BBL is:

(SEQ ID NO: 79)
MAPRRARGCRTLGLPALLLLLLLRPPATRGITCPPPMSVEHADIVKSYSLYSRERYICNSG
FKRKAGTSSLTECVLNKATNVAHWTTPSLKCIRDPALVHQRPAPPSTVTTAGVTPQPESLS
PSGKEPAASSPSSNNTAATTAAIVPGSQLMPSKSPSTGTTEISSHESSHGTPSQTTAKNWE
LTASASHQPPGVYPQGHSDTTVAISTSTVLLCGLSAVSLLACYLKSRQTPPLASVEMEAME
ALPVTWGTSSRDEDLENCSHHL.

In some embodiments, the nucleic acid sequence for HBD is:

(SEQ ID NO: 80)
GCTATCCCCGCTCCCACCGACCTGAAATTCACCCAGGTGACCCCCACCAGCCTGAGCG
CTCAATGGACCCCCCCCAATGTGCAGCTGACAGGCTACAGGGTGAGAGTGACCCCCAA
GGAAAAAACCGGACCCATGAAGGAGATCAATCTGGCCCCTGACAGCTCCAGCGTCGTG
GTCTCCGGCCTGATGGTGGCTACAAAATACGAGGTGTCCGTCTACGCCCTCAAAGATA
CCCTGACCAGCAGGCCTGCCCAAGGAGTGGTGACCACCCTGGAAAATGTCTCCCCCC
CTAGGAGAGCCAGGGTCACCGATGCCACAGAAACCACCATTACCATCTCCTGGAGGAC
CAAGACCGAAACCATTACCGGATTCCAGGTGGATGCTGTCCCCGCCAACGGACAAACA
CCTATTCAAAGGACAATCAAACCCGACGTGAGATCCTACACCATCACCGGCCTCCAGC
CCGGCACCGACTACAAAATCTATCTGTATACCCTGAACGACAACGCTAGAAGCTCCCCT
GTGGTGATCGACGCTAGCACCGCCATCGATGCCCCTAGCAATCTGAGGTTTCTGGCCA
CCACACCTAATTCCCTGCTCGTGTCCTGGCAGCCTCCCAGGGCTAGGATCACCGGCTA
CATTATCAAGTACGAGAAACCCGGCTCCCCCCCTAGAGAAGTGGTGCCTAGGCCCAGG
CCTGGAGTCACCGAGGCTACCATTACCGGCCTGGAGCCTGGAACCGAGTACACCATCT
ATGTCATCGCTCTCAAGAACAACCAGAAGTCCGAACCCCTCATTGGCAGGAAGAAGAC
CGGCGGCGGCGGCTCCGGAGGAGGCGGCTCCGGCGGCGGCGGAAGCGCTATTCCTG
CCCCTACCGATCTGAAGTTCACCCAAGTGACACCTACCTCCCTGTCCGCCCAGTGGAC
CCCCCCCAACGTGCAGCTCACCGGCTATAGGGTCAGGGTGACCCCCAAAGAGAAGAC
CGGACCCATGAAAGAGATCAACCTCGCTCCTGACTCCAGCAGCGTCGTGGTGAGCGG
CCTGATGGTGGCCACCAAGTACGAAGTGAGCGTCTACGCTCTGAAGGACACCCTGACC
AGCAGACCCGCCCAGGGAGTGGTGACCACACTGGAGAACGTCAGCCCTCCCAGAAGG
GCTAGAGTGACAGACGCCACAGAGACCACCATTACCATTAGCTGGAGGACCAAAACCG
AGACCATTACAGGATTCCAGGTCGACGCTGTCCCCGCTAACGGACAAACCCCCATCCA
GAGGACCATCAAGCCCGATGTGAGGAGCTATACCATCACCGGACTCCAACCCGGCACA
GACTACAAGATCTACCTGTACACCCTGAATGACAATGCCAGGAGCTCCCCCGTGGTCA
TTGACGCCTCCACCGCCATTGACGCTCCTAGCAACCTGAGGTTCCTGGCTACCACCCC
CAACAGCCTCCTGGTCTCCTGGCAGCCCCCCAGAGCTAGAATTACCGGATACATCATC
AAGTATGAAAAACCTGGCAGCCCCCCTAGGGAGGTCGTGCCTAGGCCTAGACCTGGC
GTGACAGAGGCTACAATTACCGGCCTGGAGCCCGGCACAGAATACACCATTTACGTCA
TCGCTCTCAAAAACAACCAGAAATCCGAGCCCCTCATCGGCAGGAAAAAGACC.

In some embodiments, the amino acid sequence for 41 BBL is:

(SEQ ID NO: 81)
MKEINLAPDSSSVVVSGLMVATKYEVSVYALKDTLTSRPAQGVVTTLENVSPPRRARVTDA
TETTITISWRTKTETITGFQVDAVPANGQTPIQRTIKPDVRSYTITGLQPGTDYKIYLYTLNDN
ARSSPVVIDASTAIDAPSNLRFLATTPNSLLVSWQPPRARITGYIIKYEKPGSPPREVVPRPR
PGVTEATITGLEPGTEYTIYVIALKNNQKSEPLIGRKKTGGGGSGGGGSGGGGSAIPAPTDL
KFTQVTPTSLSAQWTPPNVQLTGYRVRVTPKEKTGPMKEINLAPDSSSVVVSGLMVATKYE
VSVYALKDTLTSRPAQGVVTTLENVSPPRRARVTDATETTITISWRTKTETITGFQVDAVPAN
GQTPIQRTIKPDVRSYTITGLQPGTDYKIYLYTLNDNARSSPVVIDASTAIDAPSNLRFLATTP
NSLLVSWQPPRARITGYIIKYEKPGSPPREVVPRPRPGVTEATITGLEPGTEYTIYVIALKNN
QKSEPLIGRKKT.

### Engineered CD4/CD25 T Cells

T_{reg} cells express the biomarkers CD4, FOXP3, and CD25 and are thought to be derived from the same lineage as naïve CD4 cells. Because effector T cells also express CD4 and CD25, Tregs are very difficult to effectively discern from effector CD4+. In a second embodiment, the method involves isolating CD4/CD25 T cells from a subject and inducing T_{reg} differentiation in these cells through gene-modification. These cells are referred to herein as gene-induced T_{reg} (giT_{reg}) cells. In some embodiments, the isolated cells are genetically engineered to express IL2R, FOXP3, SOCS, or any combination thereof, which regulate T_{reg} differentiation.

In some embodiments, the nucleic acid sequence for FOXP3 is:

(SEQ ID NO: 82)
ATGCCCAACCCCCGGCCCGGCAAGCCCAGCGCCCCCAGCCTGGCCCTGGGCCCCAG
CCCCGGCGCCAGCCCCAGCTGGCGGGCCGCCCCCAAGGCCAGCGACCTGCTGGGCG
CCCGGGGCCCCGGCGGCACCTTCCAGGGCCGGGACCTGCGGGGCGGCGCCCACGC
CAGCAGCAGCAGCCTGAACCCCATGCCCCCCAGCCAGCTGCAGCTGCCCACCCTGCC
CCTGGTGATGGTGGCCCCCAGCGGCGCCCGGCTGGGCCCCCTGCCCCACCTGCAGG
CCCTGCTGCAGGACCGGCCCCACTTCATGCACCAGCTGAGCACCGTGGACGCCCACG
CCCGGACCCCCGTGCTGCAGGTGCACCCCCTGGAGAGCCCCGCCATGATCAGCCTGA
CCCCCCCCACCACCGCCACCGGCGTGTTCAGCCTGAAGGCCCGGCCCGGCCTGCCC
CCCGGCATCAACGTGGCCAGCCTGGAGTGGGTGAGCCGGGAGCCCGCCCTGCTGTG
CACCTTCCCCAACCCCAGCGCCCCCCGGAAGGACAGCACCCTGAGCGCCGTGCCCCA
GAGCAGCTACCCCCTGCTGGCCAACGGCGTGTGCAAGTGGCCCGGCTGCGAGAAGGT
GTTCGAGGAGCCCGAGGACTTCCTGAAGCACTGCCAGGCCGACCACCTGCTGGACGA
GAAGGGCCGGGCCCAGTGCCTGCTGCAGCGGGAGATGGTGCAGAGCCTGGAGCAGC
AGCTGGTGCTGGAGAAGGAGAAGCTGAGCGCCATGCAGGCCCACCTGGCCGGCAAGA
TGGCCCTGACCAAGGCCAGCAGCGTGGCCAGCAGCGACAAGGGCAGCTGCTGCATCG
TGGCCGCCGGCAGCCAGGGCCCCGTGGTGCCCGCCTGGAGCGGCCCCCGGGAGGC
CCCCGACAGCCTGTTCGCCGTGCGGCGGCACCTGTGGGGCAGCCACGGCAACAGCA
CCTTCCCCGAGTTCCTGCACAACATGGACTACTTCAAGTTCCACAACATGCGGCCCCC
CTTCACCTACGCCACCCTGATCCGGTGGGCCATCCTGGAGGCCCCCGAGAAGCAGCG
GACCCTGAACGAAATCTACCACTGGTTCACCCGGATGTTCGCCTTCTTCCGGAACCAC
CCCGCCACCTGGAAGAACGCCATCCGGCACAACCTGAGCCTGCACAAGTGCTTCGTG
CGGGTGGAGAGCGAGAAGGGCGCCGTGTGGACCGTGGACGAGCTGGAGTTCCGGAA
GAAGCGGAGCCAGCGGCCCAGCCGGTGCAGCAACCCCACCCCCGGCCCC.

In some embodiments, the amino acid sequence for FOXP3 is:

(SEQ ID NO: 83)
MPNPRPGKPSAPSLALGPSPGASPSWRAAPKASDLLGARGPGGTFQGRDLRGGAHASSS
SLNPMPPSQLQLPTLPLVMVAPSGARLGPLPHLQALLQDRPHFMHQLSTVDAHARTPVLQ
VHPLESPAMISLTPPTTATGVFSLKARPGLPPGINVASLEWWSREPALLCTFPNPSAPRKDS
TLSAVPQSSYPLLANGVCKWPGCEKVFEEPEDFLKHCQADHLLDEKGRAQCLLQREMVQS
LEQQLVLEKEKLSAMQAHLAGKMALTKASSVASSDKGSCCIVAAGSQGPVVPAWSGPREA
PDSLFAVRRHLWGSHGNSTFPEFLHNMDYFKFHNMRPPFTYATLIRWAILEAPEKQRTLNEN
YHWFTRMFAFFRNHPATWKNAIRHNLSLHKCFVRVESEKGAVWTVDELEFRKKRSQRPSR
CSNPTPGP.

In some embodiments, the nucleic acid sequence for IL12R is:

(SEQ ID NO: 84)
ATGGCCGCCCCCGCCCTGAGCTGGCGGCTGCCCCTGCTGATCCTGCTGCTGCCCCTG
GCCACCAGCTGGGCCAGCGCCGCCGTGAACGGCACCAGCCAGTTCACCTGCTTCTAC
AACAGCCGGGCCAACATCAGCTGCGTGTGGAGCCAGGACGGCGCCCTGCAGGACACC
AGCTGCCAGGTGCACGCCTGGCCCGACCGGCGGCGGTGGAACCAGACCTGCGAGCT
GCTGCCCGTGAGCCAGGCCAGCTGGGCCTGCAACCTGATCCTGGGCGCCCCCGACAG
CCAGAAGCTGACCACCGTGGACATCGTGACCCTGCGGGTGCTGTGCCGGGAGGGCGT
GCGGTGGCGGGTGATGGCCATCCAGGACTTCAAGCCCTTCGAGAACCTGCGGCTGAT
GGCCCCCATCAGCCTGCAGGTGGTGCACGTGGAGACCCACCGGTGCAACATCAGCTG
GGAGATCAGCCAGGCCAGCCACTACTTCGAGCGGCACCTGGAGTTCGAGGCCCGGAC
CCTGAGCCCCGGCCACACCTGGGAGGAGGCCCCCCTGCTGACCCTGAAGCAGAAGCA
GGAGTGGATCTGCCTGGAGACCCTGACCCCCGACACCCAGTACGAGTTCCAGGTGCG
GGTGAAGCCCCTGCAGGGCGAGTTCACCACCTGGAGCCCCTGGAGCCAGCCCCTGGC
CTTCCGGACCAAGCCCGCCGCCCTGGGCAAGGACACCATCCCCTGGCTGGGCCACCT
GCTGGTGGGCCTGAGCGGCGCCTTCGGCTTCATCATCCTGGTGTACCTGCTGATCAAC
TGCCGGAACACCGGCCCCTGGCTGAAGAAGGTGCTGAAGTGCAACACCCCCGACCCC
AGCAAGTTCTTCAGCCAGCTGAGCAGCGAGCACGGCGGCGACGTGCAGAAGTGGCTG
AGCAGCCCCTTCCCCAGCAGCAGCTTCAGCCCCGGCGGCCTGGCCCCCGAGATCAGC
CCCCTGGAGGTGCTGGAGCGGGACAAGGTGACCCAGCTGCTGCTGCAGCAGGACAAG
GTGCCCGAGCCCGCCAGCCTGAGCAGCAACCACAGCCTGACCAGCTGCTTCACCAAC
CAGGGCTACTTCTTCTTCCACCTGCCCGACGCCCTGGAGATCGAGGCCTGCCAGGTGT
ACTTCACCTACGACCCCTACAGCGAGGAGGACCCCGACGAGGGCGTGGCCGGCGCCC
CCACCGGCAGCAGCCCCCAGCCCCTGCAGCCCCTGAGCGGCGAGGACGACGCCTAC
TGCACCTTCCCCAGCCGGGACGACCTGCTGCTGTTCAGCCCCAGCCTGCTGGGCGGC
CCCAGCCCCCCCAGCACCGCCCCCGGCGGCAGCGGCGCCGGCGAGGAGCGGATGC
CCCCCAGCCTGCAGGAGCGGGTGCCCCGGGACTGGGACCCCCAGCCCCTGGGCCCC
CCCACCCCCGGCGTGCCCGACCTGGTGGACTTCCAGCCCCCCCCCGAGCTGGTGCTG
CGGGAGGCCGGCGAGGAGGTGCCCGACGCCGGCCCCCGGGAGGGCGTGAGCTTCC
CCTGGAGCCGGCCCCCCGGCCAGGGCGAGTTCCGGGCCCTGAACGCCCGGCTGCCC
CTGAACACCGACGCCTACCTGAGCCTGCAGGAGCTGCAGGGCCAGGACCCCACCCAC
CTGGTG.

In some embodiments, the amino acid sequence for IL12R is:

(SEQ ID NO: 85)
MAAPALSWRLPLLILLLPLATSWASAAVNGTSQFTCFYNSRANISCVWSQDGALQDTSCQV
HAWPDRRRWNQTCELLPVSQASWACNLILGAPDSQKLTTVDIVTLRVLCREGVRWRVMAI
QDFKPFENLRLMAPISLQVVHVETHRCNISWEISQASHYFERHLEFEARTLSPGHTWEEAPL
LTLKQKQEWICLETLTPDTQYEFQVRVKPLQGEFTTWSPWSQPLAFRTKPAALGKDTIPWL
GHLLVGLSGAFGFIILVYLLINCRNTGPWLKKVLKCNTPDPSKFFSQLSSEHGGDVQKWLSS
PFPSSSFSPGGLAPEISPLEVLERDKVTQLLLQQDKVPEPASLSSNHSLTSCFTNQGYFFFH
LPDALEIEACQVYFTYDPYSEEDPDEGVAGAPTGSSPQPLQPLSGEDDAYCTFPSRDDLLL
FSPSLLGGPSPPSTAPGGSGAGEERMPPSLQERVPRDWDPQPLGPPTPGVPDLVDFQPP
PELVLREAGEEVPDAGPREGVSFPWSRPPGQGEFRALNARLPLNTDAYLSLQELQGQDPT
HLV.

In some embodiments, the nucleic acid sequence for SOCS is:

(SEQ ID NO: 86)
ATGGTGACCCACAGCAAGTTCCCCGCCGCCGGCATGAGCCGGCCCCTGGACACCAGC
CTGCGGCTGAAGACCTTCAGCAGCAAGAGCGAGTACCAGCTGGTGGTGAACGCCGTG
CGGAAGCTGCAGGAGAGCGGCTTCTACTGGAGCGCCGTGACCGGCGGCGAGGCCAA
CCTGCTGCTGAGCGCCGAGCCCGCCGGCACCTTCCTGATCCGGGACAGCAGCGACCA
GCGGCACTTCTTCACCCTGAGCGTGAAGACCCAGAGCGGCACCAAGAACCTGCGGAT
CCAGTGCGAGGGCGGCAGCTTCAGCCTGCAGAGCGACCCCCGGAGCACCCAGCCCG
TGCCCCGGTTCGACTGCGTGCTGAAGCTGGTGCACCACTACATGCCCCCCCCCGGCG
CCCCCAGCTTCCCCAGCCCCCCCACCGAGCCCAGCAGCGAGGTGCCCGAGCAGCCCA
GCGCCCAGCCCCTGCCCGGCAGCCCCCCCCGGCGGGCCTACTACATCTACAGCGGC
GGCGAGAAGATCCCCCTGGTGCTGAGCCGGCCCCTGAGCAGCAACGTGGCCACCCTG
CAGCACCTGTGCCGGAAGACCGTGAACGGCCACCTGGACAGCTACGAGAAGGTGACC
CAGCTGCCCGGCCCCATCCGGGAGTTCCTGGACCAGTACGACGCCCCCCTG.

In some embodiments, the amino acid sequence for SOCS is:

(SEQ ID NO: 87)
MVTHSKFPAAGMSRPLDTSLRLKTFSSKSEYQLVVNAVRKLQESGFYWSAVTGGEANLLL
SAEPAGTFLIRDSSDQRHFFTLSVKTQSGTKNLRIQCEGGSFSLQSDPRSTQPVPRFDCVL
KLVHHYMPPPGAPSFPSPPTEPSSEVPEQPSAQPLPGSPPRRAYYIYSGGEKIPLVLSRPLS
SNVATLQHLCRKTVNGHLDSYEKVTQLPGPIREFLDQYDAPL.

### CAR Polypeptides

The disclosed methods can be used to produce chimeric antigen receptor (CAR) T_{reg} cells containing CAR polypeptides. A CAR polypeptide is generally made up of three domains: an ectodomain, a transmembrane domain, and an endodomain. The ectodomain is responsible for antigen recognition. It also optionally contains a signal peptide (SP) so that the CAR can be glycosylated and anchored in the cell membrane of the immune effector cell. The transmembrane domain (TD), is as its name suggests, connects the ectodomain to the endodomain and resides within the cell membrane when expressed by a cell. The endodomain is the business end of the CAR that transmits an activation signal to the immune effector cell after antigen recognition. For example, the endodomain can contain an intracellular signaling domain (ISD) and optionally a co-stimulatory signaling region (CSR). CAR polypeptides generally incorporate an antigen recognition domain from the single-chain variable fragments (scFv) of a monoclonal antibody (mAb) with transmembrane signaling motifs involved in lymphocyte activation (Sadelain M, et al. Nat Rev Cancer 2003 3:35-45).

A “signaling domain (SD)” generally contains immunoreceptor tyrosine-based activation motifs (ITAMs) that activate a signaling cascade when the ITAM is phosphorylated. The term “co-stimulatory signaling region (CSR)” refers to intracellular signaling domains from costimulatory protein receptors, such as CD28, 41BB, and ICOS, that are able to enhance T-cell activation by T-cell receptors.

Additional CAR constructs are described, for example, in Fresnak A D, et al. Engineered T cells: the promise and challenges of cancer immunotherapy. Nat Rev Cancer. 2016 Aug. 23; 16(9):566-81, which is incorporated by reference in its entirety for the teaching of these CAR models.

For example, the CAR can be a TRUCK, Universal CAR, Self-driving CAR, Armored CAR, Self-destruct CAR, Conditional CAR, Marked CAR, TenCAR, Dual CAR, or sCAR.

CAR T cells engineered to be resistant to immunosuppression (Armored CARs) may be genetically modified to no longer express various immune checkpoint molecules (for example, cytotoxic T lymphocyte-associated antigen 4 (CTLA4) or programmed cell death protein 1 (PD1)), with an immune checkpoint switch receptor, or may be administered with a monoclonal antibody that blocks immune checkpoint signaling.

A self-destruct CAR may be designed using RNA delivered by electroporation to encode the CAR. Alternatively, inducible apoptosis of the T cell may be achieved based on ganciclovir binding to thymidine kinase in gene-modified lymphocytes or the more recently described system of activation of human caspase 9 by a small-molecule dimerizer.

A conditional CAR T cell is by default unresponsive, or switched ‘off’, until the addition of a small molecule to complete the circuit, enabling full transduction of both signal 1 and signal 2, thereby activating the CAR T cell. Alternatively, T cells may be engineered to express an adaptor-specific receptor with affinity for subsequently administered secondary antibodies directed at target antigen.

A tandem CAR (TanCAR) T cell expresses a single CAR consisting of two linked single-chain variable fragments (scFvs) that have different affinities fused to intracellular co-stimulatory domain(s) and a CD3ζ domain. TanCAR T cell activation is achieved only when target cells co-express both targets.

A dual CAR T cell expresses two separate CARs with different ligand binding targets; one CAR includes only the CD3ζ domain and the other CAR includes only the co-stimulatory domain(s). Dual CAR T cell activation requires co-expression of both targets.

A safety CAR (sCAR) consists of an extracellular scFv fused to an intracellular inhibitory domain. sCAR T cells co-expressing a standard CAR become activated only when encountering target cells that possess the standard CAR target but lack the sCAR target.

The antigen recognition domain of the disclosed CAR is usually an scFv. There are however many alternatives. An antigen recognition domain from native T-cell receptor (TCR) alpha and beta single chains have been described, as have simple ectodomains (e.g. CD4 ectodomain to recognize HIV infected cells) and more exotic recognition components such as a linked cytokine (which leads to recognition of cells bearing the cytokine receptor). In fact almost anything that binds a given target with high affinity can be used as an antigen recognition region.

The endodomain is the business end of the CAR that after antigen recognition transmits a signal to the immune effector cell, activating at least one of the normal effector functions of the immune effector cell. Effector function of a T cell, for example, may be cytolytic activity or helper activity including the secretion of cytokines. Therefore, the endodomain may comprise the “intracellular signaling domain” of a T cell receptor (TCR) and optional co-receptors. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal.

Cytoplasmic signaling sequences that regulate primary activation of the TCR complex that act in a stimulatory manner may contain signaling motifs which are known as immunoreceptor tyrosine-based activation motifs (ITAMs). Examples of ITAM containing cytoplasmic signaling sequences include those derived from CD8, CD3ζ, CD35, CD3γ, CD3ε, CD32 (Fc gamma RIIa), DAP10, DAP12, CD79a, CD79b, FcγRly, FcγRIIIγ, FcεRIβ (FCERIB), and FcεRIγ (FCERIG).

In particular embodiments, the intracellular signaling domain is derived from CD3 zeta (CD3ζ) (TCR zeta, GenBank accno. BAG36664.1). T-cell surface glycoprotein CD3 zeta (CD3ζ) chain, also known as T-cell receptor T3 zeta chain or CD247 (Cluster of Differentiation 247), is a protein that in humans is encoded by the CD247 gene.

First-generation CARs typically had the intracellular domain from the CD3ζ chain, which is the primary transmitter of signals from endogenous TCRs. Second-generation CARs add intracellular signaling domains from various costimulatory protein receptors (e.g., CD28, 41BB, ICOS) to the endodomain of the CAR to provide additional signals to the T cell. More recent, third-generation CARs combine multiple signaling domains to further augment potency. T cells grafted with these CARs have demonstrated improved expansion, activation, persistence, and tumor-eradicating efficiency independent of costimulatory receptor/ligand interaction (Imai C, et al. Leukemia 2004 18:676-84; Maher J, et al. Nat Biotechnol 2002 20:70-5).

For example, the endodomain of the CAR can be designed to comprise the CD3ζ signaling domain by itself or combined with any other desired cytoplasmic domain(s) useful in the context of the CAR of the invention. For example, the cytoplasmic domain of the CAR can comprise a CD3ζ chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, ICOS, lymphocyte function-associated antigen-1 (LFA-1), CD2, CD7, LIGHT, NKG2C, B7-H3, and a ligand that specifically binds with CD123, CD8, CD4, b2c, CD80, CD86, DAP10, DAP12, MyD88, BTNL3, and NKG2D. Thus, while the CAR is exemplified primarily with CD28 as the co-stimulatory signaling element, other costimulatory elements can be used alone or in combination with other co-stimulatory signaling elements.

In some embodiments, the CAR comprises a hinge sequence. A hinge sequence is a short sequence of amino acids that facilitates antibody flexibility (see, e.g., Woof et al., Nat. Rev. Immunol., 4(2): 89-99 (2004)). The hinge sequence may be positioned between the antigen recognition moiety (e.g., scFv) and the transmembrane domain. The hinge sequence can be any suitable sequence derived or obtained from any suitable molecule. In some embodiments, for example, the hinge sequence is derived from a CD8a molecule or a CD28 molecule.

The transmembrane domain may be derived either from a natural or from a synthetic source. Where the source is natural, the domain may be derived from any membrane-bound or transmembrane protein. For example, the transmembrane region may be derived from (i.e. comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, or CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, and PAG/Cbp. Alternatively the transmembrane domain may be synthetic, in which case it will comprise predominantly hydrophobic residues such as leucine and valine. In some cases, a triplet of phenylalanine, tryptophan and valine will be found at each end of a synthetic transmembrane domain. A short oligo- or polypeptide linker, such as between 2 and 10 amino acids in length, may form the linkage between the transmembrane domain and the endoplasmic domain of the CAR.

In some embodiments, the CAR has more than one transmembrane domain, which can be a repeat of the same transmembrane domain, or can be different transmembrane domains.

In some embodiments, the CAR is a multi-chain CAR, as described in WO2015/039523, which is incorporated by reference for this teaching. A multi-chain CAR can comprise separate extracellular ligand binding and signaling domains in different transmembrane polypeptides. The signaling domains can be designed to assemble in juxtamembrane position, which forms flexible architecture closer to natural receptors, that confers optimal signal transduction. For example, the multi-chain CAR can comprise a part of an FCERI alpha chain and a part of an FCERI beta chain such that the FCERI chains spontaneously dimerize together to form a CAR.

In some embodiments, the antigen recognition domain is single chain variable fragment (scFv) antibody. The affinity/specificity of an scFv is driven in large part by specific sequences within complementarity determining regions (CDRs) in the heavy (V_{H}) and light (V_{L}) chain. Each V_{H} and V_{L} sequence will have three CDRs (CDR1, CDR2, CDR3).

In some embodiments, the antigen recognition domain is derived from natural antibodies, such as monoclonal antibodies. In some cases, the antibody is human. In some cases, the antibody has undergone an alteration to render it less immunogenic when administered to humans. For example, the alteration comprises one or more techniques selected from the group consisting of chimerization, humanization, CDR-grafting, deimmunization, and mutation of framework amino acids to correspond to the closest human germline sequence.

Also disclosed are bi-specific CARs that target two antigens. Also disclosed are CARs designed to work only in conjunction with another CAR that binds a different antigen. For example, in these embodiments, the endodomain of the disclosed CAR can contain only a signaling domain (SD) or a co-stimulatory signaling region (CSR), but not both. The second CAR (or endogenous T-cell) provides the missing signal if it is activated. For example, if the disclosed CAR contains an SD but not a CSR, then the immune effector cell containing this CAR is only activated if another CAR (or T-cell) containing a CSR binds its respective antigen. Likewise, if the disclosed CAR contains a CSR but not a SD, then the immune effector cell containing this CAR is only activated if another CAR (or T-cell) containing an SD binds its respective antigen.

### Therapeutic Methods

Cellular therapies with polyclonal regulatory T-cells (Tregs) in transplantation and autoimmune diseases have been carried out in both animal models and clinical trials. However, The use of large numbers of polyclonal Tregs with unknown antigen specificities has led to unwanted effects, such as systemic immunosuppression, which can be avoided via utilization of antigen-specific Tregs. Antigen-specific Tregs are also more potent in suppression than polyclonal ones. Although antigen-specific Tregs can be induced in vitro, these iTregs are usually contaminated with effector T cells during in vitro expansion. Fortunately, Tregs can be efficiently engineered with a predetermined antigen-specificity via transfection of viral vectors encoding specific T cell receptors (TCRs) or chimeric antigen receptors (CARs). Compared to Tregs engineered with TCRs (TCR-Tregs), CAR-modified Tregs (CAR-Tregs) engineered in a non-MHC restricted manner have the advantage of widespread applications, especially in transplantation and autoimmunity. CAR-Tregs also are less dependent on IL-2 than are TCR-Tregs. CAR-Tregs are promising given that they maintain stable phenotypes and functions, preferentially migrate to target sites, and exert more potent and specific immunosuppression than do polyclonal Tregs.

CAR-T_{reg} cells produced by the disclosed methods can therefore be used to treat subjects with transplantation and/or autoimmune diseases. A rapid and efficacious ex vivo production system for CAR-T_{reg} cells allows successful clinical translation of CAR-T_{reg} cells for patients with GVHD or autoimmunity.

The disclosed CAR-T_{reg} cells may be administered either alone, or as a pharmaceutical composition in combination with diluents and/or with other components such as IL-2, IL-15, or other cytokines or cell populations. Briefly, pharmaceutical compositions may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions for use in the disclosed methods are in some embodiments formulated for intravenous administration. Pharmaceutical compositions may be administered in any manner appropriate treat MM. The quantity and frequency of administration will be determined by such factors as the condition of the patient, and the severity of the patient's disease, although appropriate dosages may be determined by clinical trials.

When “an immunologically effective amount”, “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, such as 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

The administration of the disclosed compositions may be carried out in any convenient manner, including by injection, transfusion, or implantation. The compositions described herein may be administered to a patient subcutaneously, intradermally, intratumorally, intranodally, intramedullary, intramuscularly, by intravenous (i.v.) injection, or intraperitoneally. In some embodiments, the disclosed compositions are administered to a patient by intradermal or subcutaneous injection. In some embodiments, the disclosed compositions are administered by i.v. injection. The compositions may also be injected directly into a tumor, lymph node, or site of infection.

In certain embodiments, the disclosed CAR-T_{reg} cells are administered to a patient in conjunction with (e.g., before, simultaneously or following) any number of relevant treatment modalities, including but not limited to thalidomide, dexamethasone, bortezomib, and lenalidomide. In further embodiments, the CAR-modified immune effector cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. In some embodiments, the CAR-modified immune effector cells are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in some embodiments, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

The cancer of the disclosed methods can be any TAA-expressing cell in a subject undergoing unregulated growth, invasion, or metastasis. In some aspects, the cancer can be any neoplasm or tumor for which radiotherapy is currently used. Alternatively, the cancer can be a neoplasm or tumor that is not sufficiently sensitive to radiotherapy using standard methods. Thus, the cancer can be a sarcoma, lymphoma, leukemia, carcinoma, blastoma, or germ cell tumor. A representative but non-limiting list of cancers that the disclosed compositions can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, endometrial cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

The disclosed CAR-T_{reg} cells can be used in combination with any compound, moiety or group which has a cytotoxic or cytostatic effect. Drug moieties include chemotherapeutic agents, which may function as microtubulin inhibitors, mitosis inhibitors, topoisomerase inhibitors, or DNA intercalators, and particularly those which are used for cancer therapy.

The disclosed CAR-T_{reg} cells can be used in combination with a checkpoint inhibitor. The two known inhibitory checkpoint pathways involve signaling through the cytotoxic T-lymphocyte antigen-4 (CTLA-4) and programmed-death 1 (PD-1) receptors. These proteins are members of the CD28-B7 family of cosignaling molecules that play important roles throughout all stages of T cell function. The PD-1 receptor (also known as CD279) is expressed on the surface of activated T cells. Its ligands, PD-L1 (B7-H1; CD274) and PD-L2 (B7-DC; CD273), are expressed on the surface of APCs such as dendritic cells or macrophages. PD-L1 is the predominant ligand, while PD-L2 has a much more restricted expression pattern. When the ligands bind to PD-1, an inhibitory signal is transmitted into the T cell, which reduces cytokine production and suppresses T-cell proliferation. Checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

Human monoclonal antibodies to programmed death 1 (PD-1) and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

In some embodiments, the PDL1 inhibitor comprises an antibody that specifically binds PDL1, such as BMS-936559 (Bristol-Myers Squibb) or MPDL3280A (Roche). In some embodiments, the PD1 inhibitor comprises an antibody that specifically binds PD1, such as lambrolizumab (Merck), nivolumab (Bristol-Myers Squibb), or MED14736 (AstraZeneca). Human monoclonal antibodies to PD-1 and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

The disclosed CAR-T_{reg} cells can be used in combination with other cancer immunotherapies. There are two distinct types of immunotherapy: passive immunotherapy uses components of the immune system to direct targeted cytotoxic activity against cancer cells, without necessarily initiating an immune response in the patient, while active immunotherapy actively triggers an endogenous immune response. Passive strategies include the use of the monoclonal antibodies (mAbs) produced by B cells in response to a specific antigen. The development of hybridoma technology in the 1970s and the identification of tumor-specific antigens permitted the pharmaceutical development of mAbs that could specifically target tumor cells for destruction by the immune system. Thus far, mAbs have been the biggest success story for immunotherapy; the top three best-selling anticancer drugs in 2012 were mAbs. Among them is rituximab (Rituxan, Genentech), which binds to the CD20 protein that is highly expressed on the surface of B cell malignancies such as non-Hodgkin's lymphoma (NHL). Rituximab is approved by the FDA for the treatment of NHL and chronic lymphocytic leukemia (CLL) in combination with chemotherapy. Another important mAb is trastuzumab (Herceptin; Genentech), which revolutionized the treatment of HER2 (human epidermal growth factor receptor 2)-positive breast cancer by targeting the expression of HER2.

Generating optimal “killer” CD8 T cell responses also requires T cell receptor activation plus co-stimulation, which can be provided through ligation of tumor necrosis factor receptor family members, including OX40 (CD134) and 4-1BB (CD137). OX40 is of particular interest as treatment with an activating (agonist) anti-OX40 mAb augments T cell differentiation and cytolytic function leading to enhanced anti-tumor immunity against a variety of tumors.

In some embodiments, such an additional therapeutic agent may be selected from an antimetabolite, such as methotrexate, 6-mercaptopurine, 6-thioguanine, cytarabine, fludarabine, 5-fluorouracil, decarbazine, hydroxyurea, asparaginase, gemcitabine or cladribine.

In some embodiments, such an additional therapeutic agent may be selected from an alkylating agent, such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin.

In some embodiments, such an additional therapeutic agent may be selected from an anti-mitotic agent, such as taxanes, for instance docetaxel, and paclitaxel, and vinca alkaloids, for instance vindesine, vincristine, vinblastine, and vinorelbine.

In some embodiments, such an additional therapeutic agent may be selected from a topoisomerase inhibitor, such as topotecan or irinotecan, or a cytostatic drug, such as etoposide and teniposide.

In some embodiments, such an additional therapeutic agent may be selected from a growth factor inhibitor, such as an inhibitor of ErbBI (EGFR) (such as an EGFR antibody, e.g. zalutumumab, cetuximab, panitumumab or nimotuzumab or other EGFR inhibitors, such as gefitinib or erlotinib), another inhibitor of ErbB2 (HER2/neu) (such as a HER2 antibody, e.g. trastuzumab, trastuzumab-DM I or pertuzumab) or an inhibitor of both EGFR and HER2, such as lapatinib).

In some embodiments, such an additional therapeutic agent may be selected from a tyrosine kinase inhibitor, such as imatinib (Glivec, Gleevec ST1571) or lapatinib.

Therefore, in some embodiments, a disclosed antibody is used in combination with ofatumumab, zanolimumab, daratumumab, ranibizumab, nimotuzumab, panitumumab, hu806, daclizumab (Zenapax), basiliximab (Simulect), infliximab (Remicade), adalimumab (Humira), natalizumab (Tysabri), omalizumab (Xolair), efalizumab (Raptiva), and/or rituximab.

In some embodiments, a therapeutic agent for use in combination with a CARs for treating the disorders as described above may be an anti-cancer cytokine, chemokine, or combination thereof. Examples of suitable cytokines and growth factors include IFNy, IL-2, IL-4, IL-6, IL-7, IL-10, IL-12, IL-13, IL-15, IL-18, IL-23, IL-24, IL-27, IL-28a, IL-28b, IL-29, KGF, IFNa (e.g., INFa2b), IFN, GM-CSF, CD40L, Flt3 ligand, stem cell factor, ancestim, and TNFa. Suitable chemokines may include Glu-Leu-Arg (ELR)-negative chemokines such as IP-10, MCP-3, MIG, and SDF-la from the human CXC and C-C chemokine families. Suitable cytokines include cytokine derivatives, cytokine variants, cytokine fragments, and cytokine fusion proteins.

In some embodiments, a therapeutic agent for use in combination with CAR-T_{reg} cells for treating the disorders as described above may be a cell cycle control/apoptosis regulator (or “regulating agent”). A cell cycle control/apoptosis regulator may include molecules that target and modulate cell cycle control/apoptosis regulators such as (i) cdc-25 (such as NSC 663284), (ii) cyclin-dependent kinases that overstimulate the cell cycle (such as flavopiridol (L868275, HMR1275), 7-hydroxystaurosporine (UCN-01, KW-2401), and roscovitine (R-roscovitine, CYC202)), and (iii) telomerase modulators (such as BIBR1532, SOT-095, GRN163 and compositions described in for instance U.S. Pat. Nos. 6,440,735 and 6,713,055). Non-limiting examples of molecules that interfere with apoptotic pathways include TNF-related apoptosis-inducing ligand (TRAIL)/apoptosis-2 ligand (Apo-2L), antibodies that activate TRAIL receptors, IFNs, and anti-sense Bcl-2.

In some embodiments, a therapeutic agent for use in combination with a CAR-T_{reg} cells for treating the disorders as described above may be a hormonal regulating agent, such as agents useful for anti-androgen and anti-estrogen therapy. Examples of such hormonal regulating agents are tamoxifen, idoxifene, fulvestrant, droloxifene, toremifene, raloxifene, diethylstilbestrol, ethinyl estradiol/estinyl, an antiandrogene (such as flutaminde/eulexin), a progestin (such as such as hydroxyprogesterone caproate, medroxy-progesterone/provera, megestrol acepate/megace), an adrenocorticosteroid (such as hydrocortisone, prednisone), luteinizing hormone-releasing hormone (and analogs thereof and other LHRH agonists such as buserelin and goserelin), an aromatase inhibitor (such as anastrazole/arimidex, aminoglutethimide/cytraden, exemestane) or a hormone inhibitor (such as octreotide/sandostatin).

In some embodiments, a therapeutic agent for use in combination with CAR-T_{reg} cells for treating the disorders as described above may be an anti-cancer nucleic acid or an anti-cancer inhibitory RNA molecule.

Combined administration, as described above, may be simultaneous, separate, or sequential. For simultaneous administration the agents may be administered as one composition or as separate compositions, as appropriate.

In some embodiments, the disclosed CAR-T_{reg} cells are administered in combination with radiotherapy. Radiotherapy may comprise radiation or associated administration of radiopharmaceuticals to a patient is provided. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, and indium-111.

In some embodiments, the disclosed CAR-T_{reg} cells are administered in combination with surgery.

CAR-T cells may be designed in several ways that enhance tumor cytotoxicity and specificity, evade tumor immunosuppression, avoid host rejection, and prolong their therapeutic half-life. TRUCK (T-cells Redirected for Universal Cytokine Killing) T cells for example, possess a CAR but are also engineered to release cytokines such as IL-12 that promote tumor killing. Because these cells are designed to release a molecular payload upon activation of the CAR once localized to the tumor environment, these CAR-T cells are sometimes also referred to as ‘armored CARs’. Several cytokines as cancer therapies are being investigated both pre-clinically and clinically, and may also prove useful when similarly incorporated into a TRUCK form of CAR-T therapy. Among these include IL-2, IL-3. IL-4, IL-5, IL-6, IL-7, IL-10, IL-12, IL-13, IL-15, IL-18, M-CSF, GM-CSF, IFN-α, IFN-γ, TNF-α, TRAIL, FLT3 ligand, Lymphotactin, and TGF-β (Dranoff 2004). “Self-driving” or “homing” CAR-T cells are engineered to express a chemokine receptor in addition to their CAR. As certain chemokines can be upregulated in tumors, incorporation of a chemokine receptor aids in tumor trafficking to and infiltration by the adoptive T-cell, thereby enhancing both specificity and functionality of the CAR-T (Moon 2011). Universal CAR-T cells also possess a CAR, but are engineered such that they do not express endogenous TCR (T-cell receptor) or MHC (major histocompatibility complex) proteins. Removal of these two proteins from the signaling repertoire of the adoptive T-cell therapy prevents graft-versus-host-disease and rejection, respectively. Armored CAR-T cells are additionally so named for their ability to evade tumor immunosuppression and tumor-induced CAR-T hypofunction. These particular CAR-Ts possess a CAR, and may be engineered to not express checkpoint inhibitors. Alternatively, these CAR-Ts can be co-administered with a monoclonal antibody (mAb) that blocks checkpoint signaling. Administration of an anti-PDL1 antibody significantly restored the killing ability of CAR TILs (tumor infiltrating lymphocytes). While PD1-PDL1 and CTLA-4-CD80/CD86 signaling pathways have been investigated, it is possible to target other immune checkpoint signaling molecules in the design of an armored CAR-T including LAG-3, Tim-3, IDO-1, 2B4, and KIR. Other intracellular inhibitors of TILs include phosphatases (SHP1), ubiquitin-ligases (i.e., cbl-b), and kinases (i.e., diacylglycerol kinase). Armored CAR-Ts may also be engineered to express proteins or receptors that protect them against or make them resistant to the effects of tumor-secreted cytokines. For example, CTLs (cytotoxic T lymphocytes) transduced with the double negative form of the TGF-β receptor are resistant to the immunosuppression by lymphoma secreted TGF-β. These transduced cells showed notably increased antitumor activity in vivo when compared to their control counterparts.

Tandem and dual CAR-T cells are unique in that they possess two distinct antigen binding domains. A tandem CAR contains two sequential antigen binding domains facing the extracellular environment connected to the intracellular costimulatory and stimulatory domains. A dual CAR is engineered such that one extracellular antigen binding domain is connected to the intracellular costimulatory domain and a second, distinct extracellular antigen binding domain is connected to the intracellular stimulatory domain. Because the stimulatory and costimulatory domains are split between two separate antigen binding domains, dual CARs are also referred to as “split CARs”. In both tandem and dual CAR designs, binding of both antigen binding domains is necessary to allow signaling of the CAR circuit in the T-cell. Because these two CAR designs have binding affinities for different, distinct antigens, they are also referred to as “bi-specific” CARs.

One primary concern with CAR-T cells as a form of “living therapeutic” is their manipulability in vivo and their potential immune-stimulating side effects. To better control CAR-T therapy and prevent against unwanted side effects, a variety of features have been engineered including off-switches, safety mechanisms, and conditional control mechanisms. Both self-destruct and marked/tagged CAR-T cells for example, are engineered to have an “off-switch” that promotes clearance of the CAR-expressing T-cell. A self-destruct CAR-T contains a CAR, but is also engineered to express a pro-apoptotic suicide gene or “elimination gene” inducible upon administration of an exogenous molecule. A variety of suicide genes may be employed for this purpose, including HSV-TK (herpes simplex virus thymidine kinase), Fas, iCasp9 (inducible caspase 9), CD20, MYC tag, and truncated EGFR (endothelial growth factor receptor). HSK for example, will convert the prodrug ganciclovir (GCV) into GCV-triphosphate that incorporates itself into replicating DNA, ultimately leading to cell death. iCasp9 is a chimeric protein containing components of FK506-binding protein that binds the small molecule AP1903, leading to caspase 9 dimerization and apoptosis. A marked/tagged CAR-T cell however, is one that possesses a CAR but also is engineered to express a selection marker. Administration of a mAb against this selection marker will promote clearance of the CAR-T cell. Truncated EGFR is one such targetable antigen by the anti-EGFR mAb, and administration of cetuximab works to promotes elimination of the CAR-T cell. CARs created to have these features are also referred to as sCARs for ‘switchable CARs’, and RCARs for ‘regulatable CARs’. A “safety CAR”, also known as an “inhibitory CAR” (iCAR), is engineered to express two antigen binding domains. One of these extracellular domains is directed against a tumor related antigen and bound to an intracellular costimulatory and stimulatory domain. The second extracellular antigen binding domain however is specific for normal tissue and bound to an intracellular checkpoint domain such as CTLA4, PD1, or CD45. Incorporation of multiple intracellular inhibitory domains to the iCAR is also possible. Some inhibitory molecules that may provide these inhibitory domains include B7-H1, B7-1, CD160, PIH, 2B4, CEACAM (CEACAM-1. CEACAM-3, and/or CEACAM-5), LAG-3, TIGIT, BTLA, LAIR1, and TGFβ-R. In the presence of normal tissue, stimulation of this second antigen binding domain will work to inhibit the CAR. It should be noted that due to this dual antigen specificity, iCARs are also a form of bi-specific CAR-T cells. The safety CAR-T engineering enhances specificity of the CAR-T cell for tumor tissue, and is advantageous in situations where certain normal tissues may express very low levels of a tumor associated antigen that would lead to off target effects with a standard CAR (Morgan 2010). A conditional CAR-T cell expresses an extracellular antigen binding domain connected to an intracellular costimulatory domain and a separate, intracellular costimulator. The costimulatory and stimulatory domain sequences are engineered in such a way that upon administration of an exogenous molecule the resultant proteins will come together intracellularly to complete the CAR circuit. In this way, CAR-T activation can be modulated, and possibly even ‘fine-tuned’ or personalized to a specific patient. Similar to a dual CAR design, the stimulatory and costimulatory domains are physically separated when inactive in the conditional CAR; for this reason these too are also referred to as a “split CAR”.

In some embodiments, two or more of these engineered features may be combined to create an enhanced, multifunctional CAR-T. For example, it is possible to create a CAR-T cell with either dual- or conditional-CAR design that also releases cytokines like a TRUCK. In some embodiments, a dual-conditional CAR-T cell could be made such that it expresses two CARs with two separate antigen binding domains against two distinct cancer antigens, each bound to their respective costimulatory domains. The costimulatory domain would only become functional with the stimulatory domain after the activating molecule is administered. For this CAR-T cell to be effective the cancer must express both cancer antigens and the activating molecule must be administered to the patient; this design thereby incorporating features of both dual and conditional CAR-T cells.

Typically, CAR-T cells are created using α-β T cells, however γ-δ T cells may also be used. In some embodiments, the described CAR constructs, domains, and engineered features used to generate CAR-T cells could similarly be employed in the generation of other types of CAR-expressing immune cells including NK (natural killer) cells, B cells, mast cells, myeloid-derived phagocytes, and NKT cells. Alternatively, a CAR-expressing cell may be created to have properties of both T-cell and NK cells. In an additional embodiment, the transduced with CARs may be autologous or allogeneic.

Several different methods for CAR expression may be used including retroviral transduction (including γ-retroviral), lentiviral transduction, transposon/transposases (Sleeping Beauty and PiggyBac systems), and messenger RNA transfer-mediated gene expression. Gene editing (gene insertion or gene deletion/disruption) has become of increasing importance with respect to the possibility for engineering CAR-T cells as well. CRISPR-Cas9, ZFN (zinc finger nuclease), and TALEN (transcription activator like effector nuclease) systems are three potential methods through which CAR-T cells may be generated.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Cell-Based AAPC System for Rapidly Expanding Gene-Engineered T Cells

Ex vivo production of Tregs can be lengthy, cumbersome, expensive, and/or inefficacious. A cell-based AAPC system for rapidly expanding gene-engineered T cells was developed. A previous system used host-derived dendritic cells to present alloantigens to recipient-derived Tregs, which was an important innovation because it allowed production of antigen-specific Tregs (Veerapathran A, et al. Blood. 2013 122(13):2251-61). However, targeting CD83+ alloreactive T cells with a CAR allows development of a large population of antigen-specific Tregs by gene-transfer. Therefore, the focus with the AAPC was to use them to support gene-modification and expansion of Tregs. The rationale is that a rapid and efficacious ex vivo production system for Tregs allows successful clinical translation of Tregs for patients with GVHD or autoimmunity.

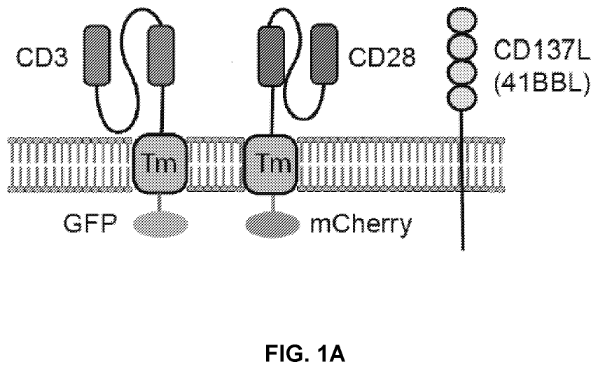

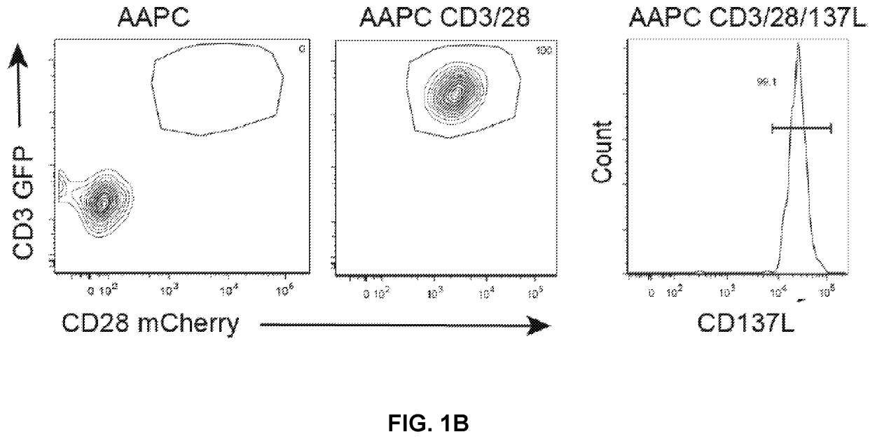

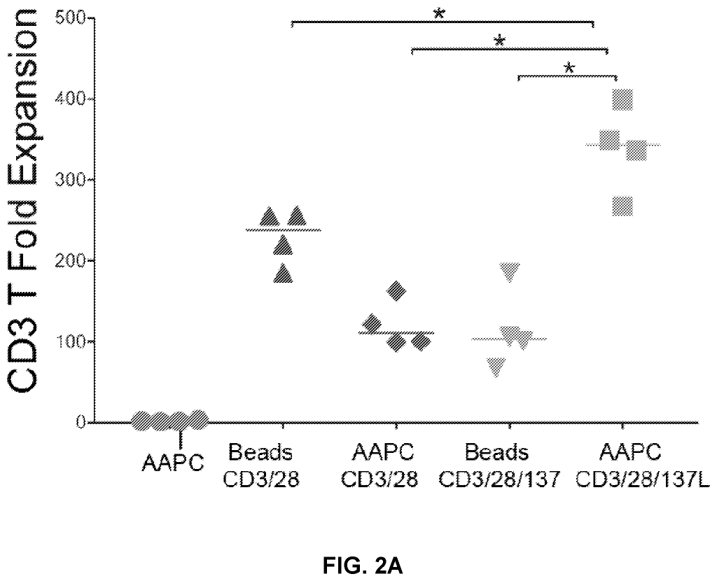

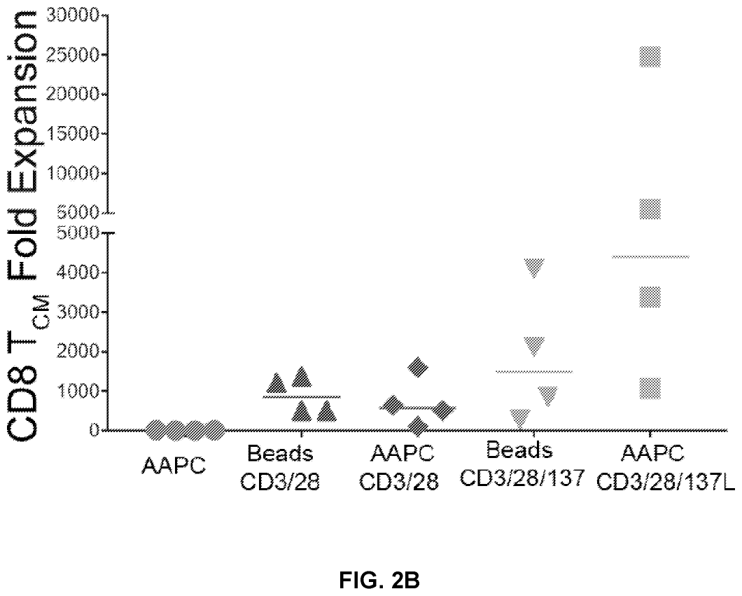

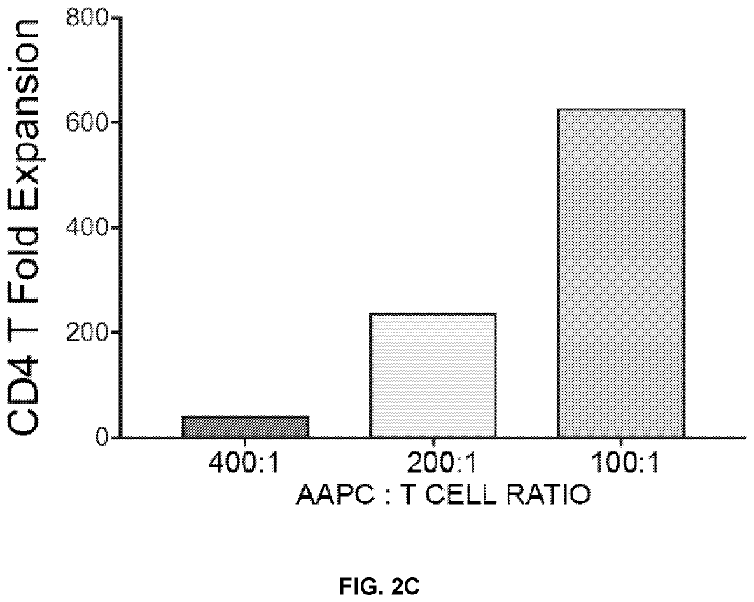

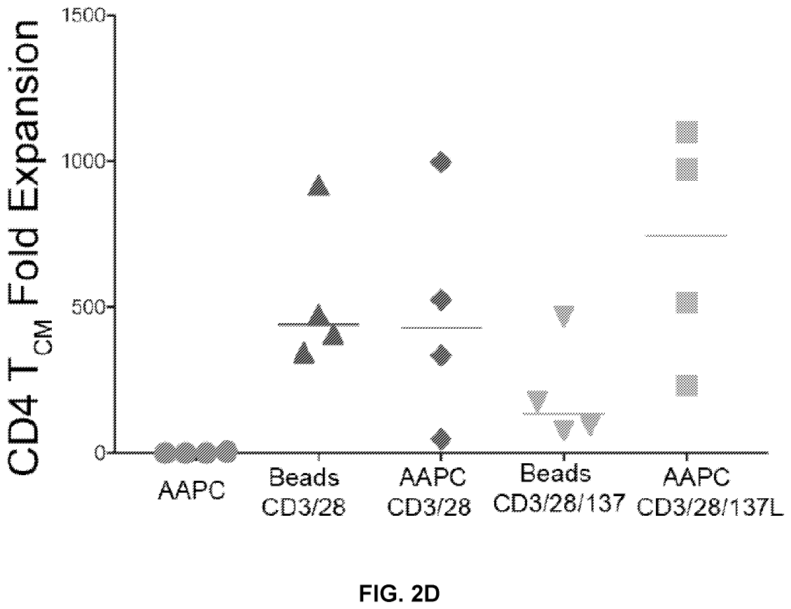

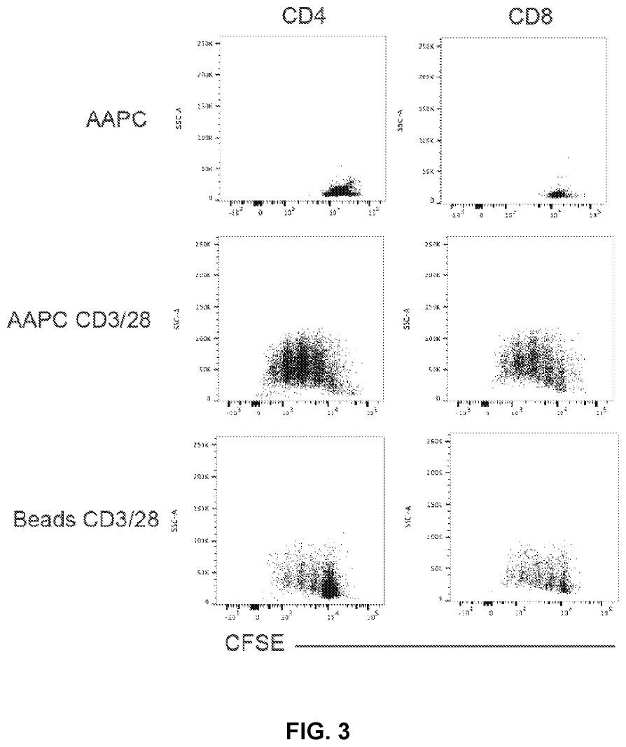

AAPCs were created to provide primary activation via CD3ζ and co-stimulation via CD28 (FIG. 1). This was accomplished by cloning IgH and lgL rearrangements associated with an anti-CD3 antibody (OKT3) and an anti-CD28 antibody (YTH 913.12). anti-CD3 and anti-CD28 scFv's from these IgH and lgL rearrangements were designed, paired with a reporter, and cloned into the SFG gammaretroviral construct. The human K562 cell line was transduced with these constructs as well as the 41 BB ligand (41 BBL), which enhance CD8 T cell memory expansion, as well as Treg expansion. AAPC stimulated proliferation of T cells collected from healthy donors up to 400-fold. This was almost 2× greater than T cell proliferation after stimulation with CD3/C28 magnetic beads, which are often used for clinical expansion of T cells and/or Tregs (FIG. 2).

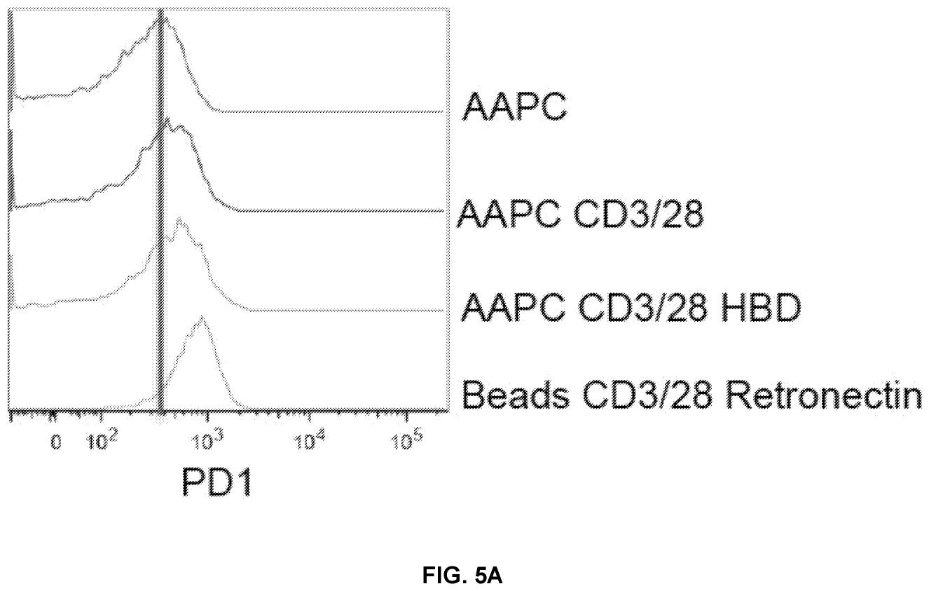

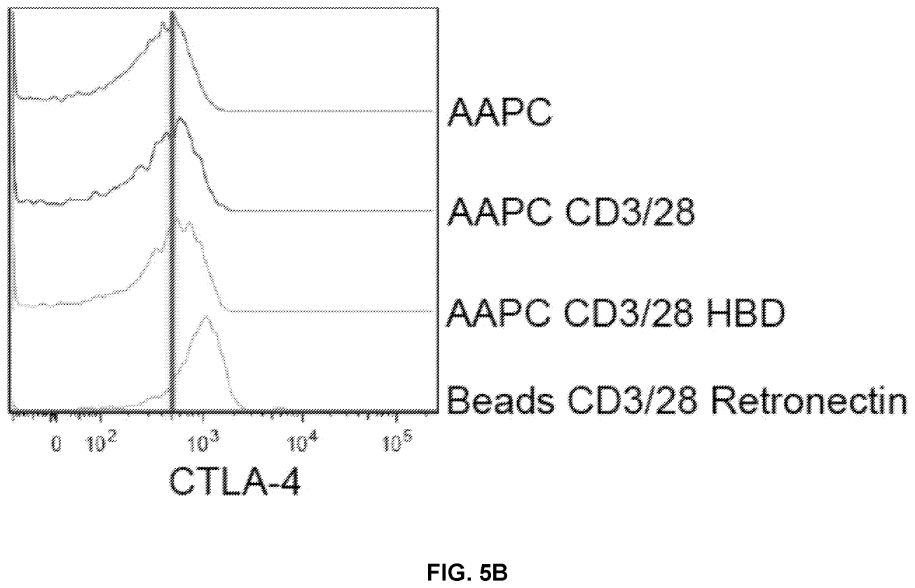

The proliferation of CD4 and CD8 T cells was also examined via CFSE. AAPC activated T cells had similar proliferation compared to bead activated T cells. AAPC was further modified to provide IL15 costimulation, which supports Treg expansion, by cloning in the IL15 receptor (IL15R). To facilitate retroviral gene transfer by using AAPC the K562 cell line was also modified to express the heparin-binding domain (HBD), which binds retrovirus (FIG. 4). The AAPC that ligate CD3 and bind virus via HBD support efficient gene transfer of primary human T cells (FIG. 4). Different methods for expressing HBD within the AAPC were evaluated, two that are expressed as part of the scFv (AAPC/GE I and II) and a third optimal design that expresses the HBD as a separate protein. The level of gene-transfer with AAPC approaches that of the gold-standard CD3/CD28 beads and retronectin. The exhaustion level of T cells stimulated with AAPC vs. beads was also examined, showing that AAPC activated T cells had lower levels of exhaustion compared to the bead activated T cells (FIG. 5).

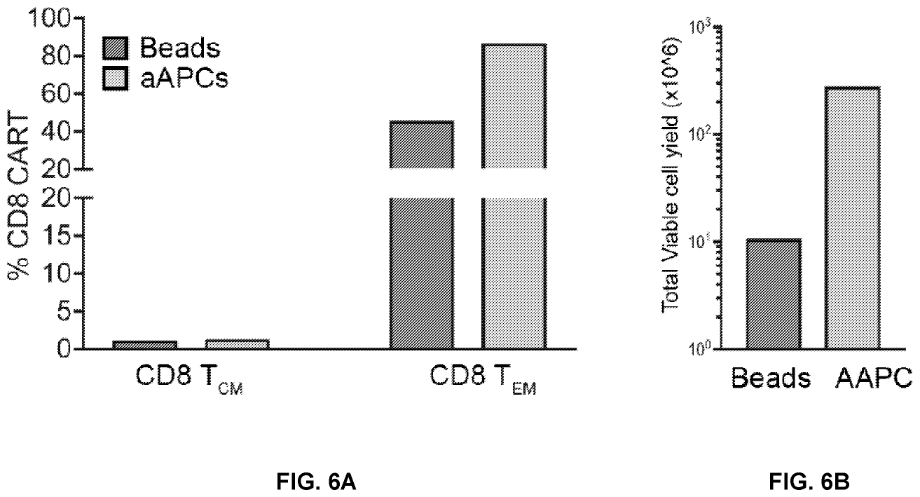

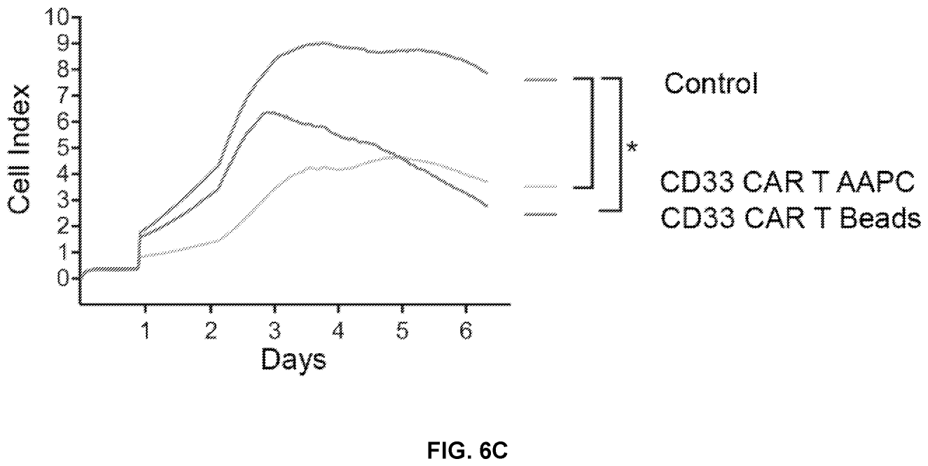

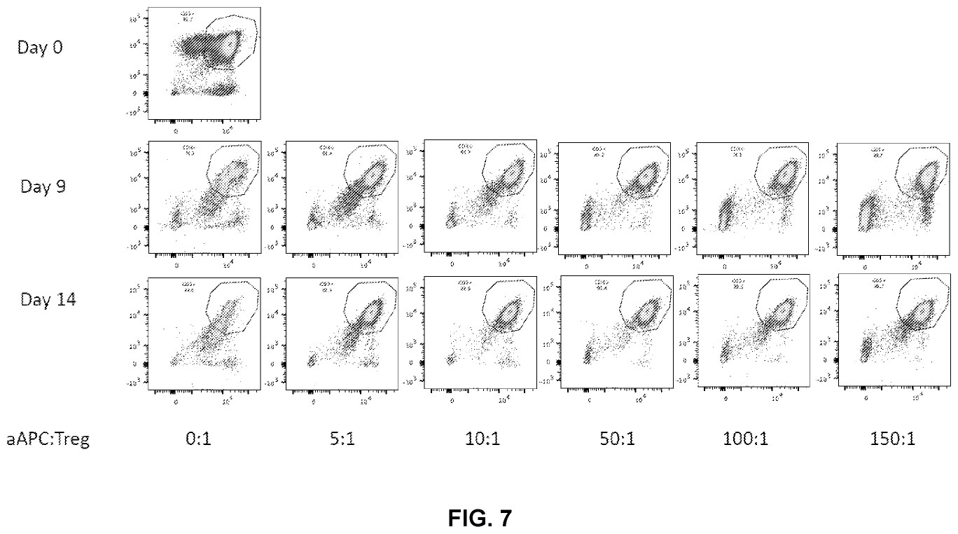

Also, production of gene-modified T cells with our AAPC is scalable for clinical use (FIG. 6). And the AAPC can be used to gene-engineer Tregs with the anti-CD83 CAR (FIG. 7) and support their expansion (FIG. 7). Data demonstrate the feasibility of the objective to develop an optimized CAR Treg production process using AAPC.

### Example 2: Test the Production Efficacy of CD83 CAR Tregs

A previous clinical evaluation of Tregs in patients relied on host-derived dendritic cell presentation of alloantigens for antigen-specific activation of recipient derived Tregs (Veerapathran A, et al. Blood. 2013 122(13):2251-61). However, this system was costly, lengthy, and somewhat inefficient. Since CD83 provides targeting to alloreactive T cells we are genetically engineering antigen-specificity and will use AAPC to gene-engineer and expand this antigen-specific Tregs. Experiments are conducted to evaluate how rapidly AAPC, and at what AAPC:Treg ratio, an optimal number of efficacious Tregs is produced. Treg function is evaluated in vitro by cytokine and proliferation suppression assays and in vivo by prevention of GVHD in mice.

Experiments are conducted to evaluate how rapidly AAPC, and at what AAPC:Treg ratio and IL2 design, an optimal number of efficacious Tregs is produced. 1×10⁵ Tregs are isolate from the PBMC of donors. The Tregs are cultured with IL2 at 300 IU/mL and irradiated (5,000 cGy) K562 AAPC that express anti-CD3, anti-CD28, 41BBL, IL15R, and the HBD at a AAPC:TIL ratio of 200:1, 100:1, 50:1, and 25:1 in Grex flasks. On Day 7 the Tregs are co-cultured with another dose of AAPC (200:1, 100:1, 50:1, and 25:1) and expanded for 7 more days for a total production time of 14 days. There are 3 comparison groups: 1) no stim as a negative control, 2) anti-CD3/CD28 beads, and 3) our system of host-derived dendritic cell presentation of alloantigens for antigen-specific activation of recipient derived Tregs for expansion (Veerapathran A, et al. Blood. 2013 122(13):2251-61). total Treg cell yields, as well as immune phenotypes, are compared. In addition, in vitro antigen-specific proliferation, cytokine, and suppression are compared. Once optimal AAPC:Treg ratios are identified, the IL2 dosage is varied from 100, 300, 1000, and 3000 IU/mL and Treg production compare again. A greater yield of Tregs with AAPC is produced. Tregs also have optimal functional characteristics of cytokine production and cytotoxic T cell suppression.

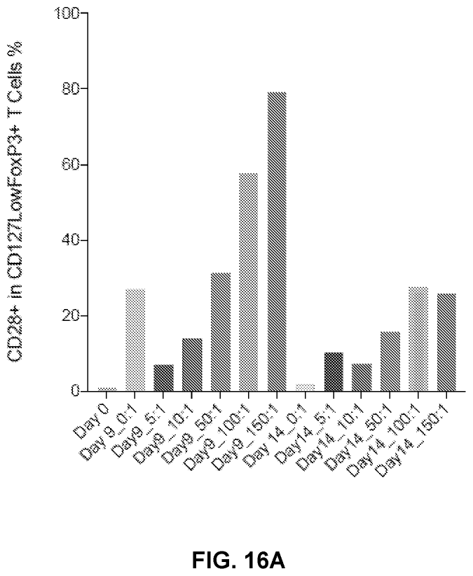

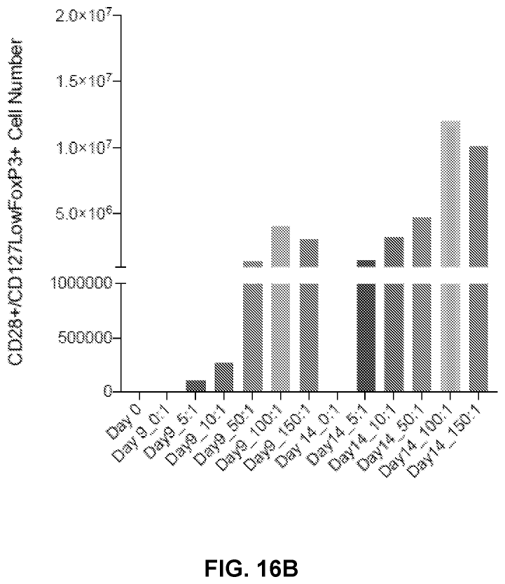

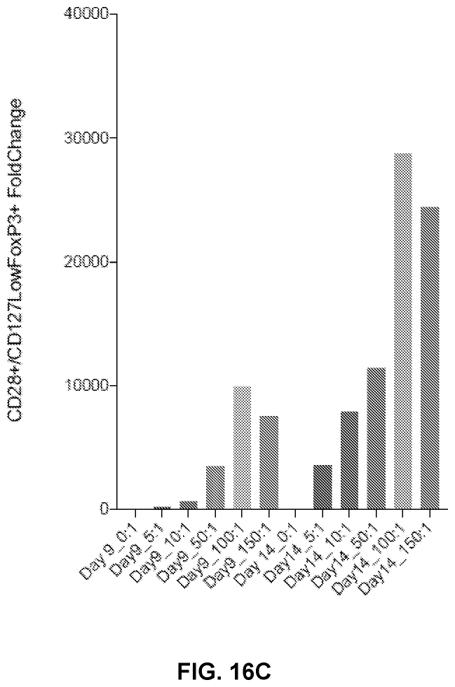

The optimal conditions for CAR Treg production is identified. The protocol that we developed in FIG. 16 call for T cell activation with AAPC for 5 days. On days 4-5 the Tregs are incubated and spinoculated with anti-CD83 CAR gamma-retrovirus following protocols that we have published (Li G, et al. Methods Mol Biol. 2017 1514:111-8) and on Day 7 another dose of AAPC is provided and the CAR T cells expanded over the following week and evaluate CAR T cells on Day 14. Optimization included comparison of the AAPC that include the HBD in cis (I and II) or in trans (Ill). A determination is made of what configuration of HBD in the AAPC supports the highest level of CAR transduction and CAR Treg cell expansion. As a control, CAR transduction and expansion over 14 days is compared to the standard method using CD3/CD28 beads (Hollyman D, et al. J Immunother. 2009 32(2):169-80; Davila M L, et al. Sci Transl Med. 2014 6(224):224ra25). All productions are performed in biologic triplicates and repeated two times with T cells collected from other healthy donors to confirm results.

### Example 3: Induce a Treg Phenotype Through CD4/CD25 T Cell Gene-Engineering

Another method to create a large population of T_{reg} cells is by inducing T_{reg} differentiation through gene-modification. CD4/CD25 T cells are isolated and gene-engineered to express IL2RB and/or FOXP3, and/or SOCS, which regulate T_{reg} differentiation. An evaluation and comparison is made of gene-induced T_{reg} phenotype to Tregs produced by host-derived dendritic cell presentation of alloantigens to recipient derived Tregs. This analysis includes gene-expression, immune phenotyping, and in vitro and in vivo functional assays.

An evaluation and comparison is made of gene-induced T_{reg} (giT_{reg}) phenotype to T_{reg} cells produced by hostderived dendritic cell presentation of alloantigens to recipient derived T_{reg} cells. Another method to create a large population of T_{reg} cells is by inducing Treg differentiation through gene-modification. IL2RB, FOXP3, and SOCS have all been shown to be critical for inducing or maintain the T_{reg} phenotype differentiation. Therefore, to determine if one can rapidly produce a large number of T_{reg} cells by gene-engineering CD4+CD25+ T cells are isolate (Veerapathran A, et al. Blood. 2013 122(13):2251-61). However, the CD4/CD25 T cells are modified by transduction with IL2RB, FOXP3, or SOCS. This is accomplished by cloning the Il2rb, foxp3, and socs genes individually into a SFG retroviral vector (SFG-il2rb, SFG-foxp3, SFG-socs). Virus is then produced and the CD4/CD25 T cells gene-modified by spinoculation after CD3/CD28 bead activation (Li G, et al. Methods Mol Biol. 2017 1514:111-8). Control groups are mock transduced CD4/CD25 T cells (negative control) and (positive control) Tregs produced by host-derived dendritic cell presentation of alloantigens by recipient derived Tregs as described (Veerapathran A, et al. Blood. 2013 122(13):2251-61). All productions occur from 14-21 days and include IL2 at 300 IU/mL. To evaluate the function of the conventional and giT_{reg} cells, their gene-expression, immune phenotype including intracellular foxp3, and ability to suppress cytotoxic T cell function in vitro in a MLR assay are compared. Their in vivo function is also compared by determining how well they prevent GVHD in an allogeneic PBMC adoptive transfer into NSG mice as described (FIG. 5).

Considering that modification of CD4/CD25 T cells with IL2RB, FOXP3, or SOCS may alter the function of T_{reg} cells, the function of CD83-targeted CARs is compared with different co-stimulatory domains. 2nd generation CD83 CARs are designed with a CD28, CD27, OX40, ICOS, or 41BB domain. these CAR constructs are combined with the giT_{reg} constructs to allow production of CD83 CAR giT_{reg} cells. Control groups are conventional T_{reg} cells (negative control) and CD83 CAR conventional T_{reg} cells (positive control). All productions will occur from 14-21 days and include CD3/CD28 bead expansion and IL2 at 300 IU/mL. Their gene-expression is compared by RNA-SEQ, immune phenotype, and ability to suppress cytotoxic T cell function in vitro in a MLR assay.

A GMP-grade AAPC cell line is produced. This GMP-grade cell line is used to validate a scaled-up GMP protocol for CAR giT_{reg} production. Modified GMP-protocol is scale up and validated starting from a leukapheresis product collected from a healthy donor. CD4/CD25 T cells are isolate from the pheresis using the CTF's MACs enrichment columns. These CD4/CD25 T cells are modified with a bi-cistronic SFG retrorviral construct that includes both CD83 CAR and socs, il2rb, and/or foxp3. The yield, gene-transfer, and in vitro function are evaluated, which includes antigen-specific cytokine-production and cytotoxic T cell suppression in an MLR assay. Finally, sterility testing is performed as part of a certificate of analyses. This protocol is valided by repeating the process with 2 other leukapheresis products to include a total of 3 scaled-up CAR T cell productions.

### Example 4: Human Treg Expansion with Quadruplet aAPCs

FIG. 7 shows CD3 in live cells at day 0, 9, and 14 with aAPC:Treg at 0:1, 5:1, 10:1, 50:1, 100:1, and 150:1.

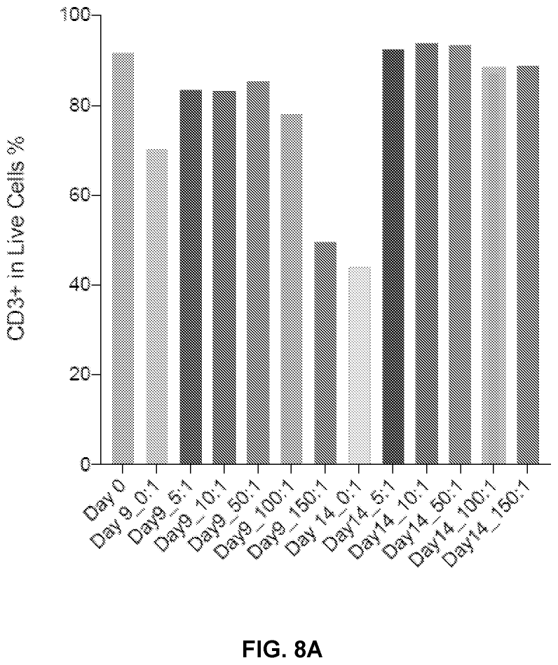

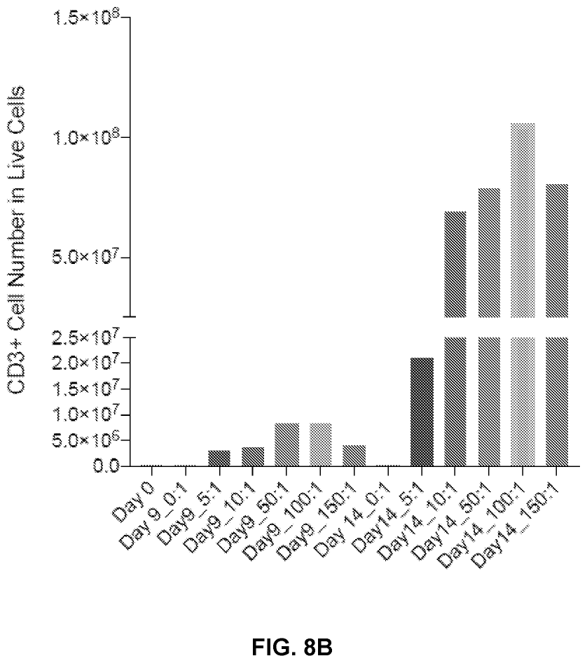

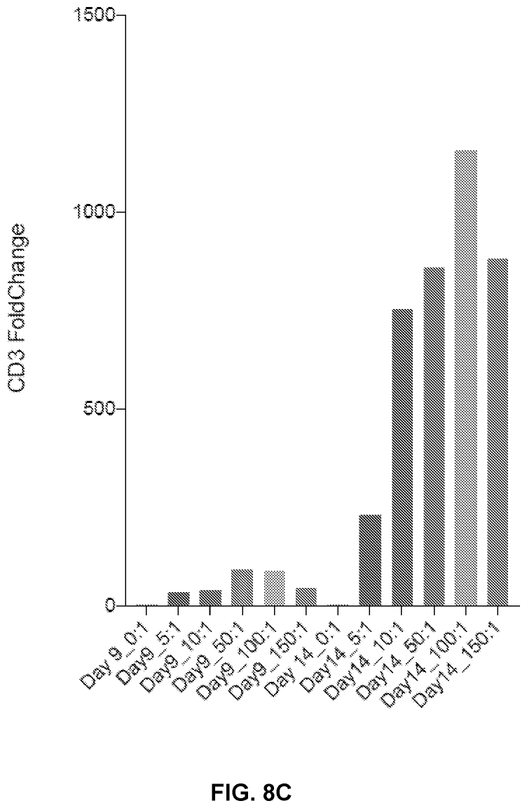

FIGS. 8A to 8C show CD3 percentage (FIG. 8A), number (FIG. 8B), and fold change (FIG. 8C) in live cells at day 0, 9, and 14 with aAPC:Treg at 0:1, 5:1, 10:1, 50:1, 100:1, and 150:1

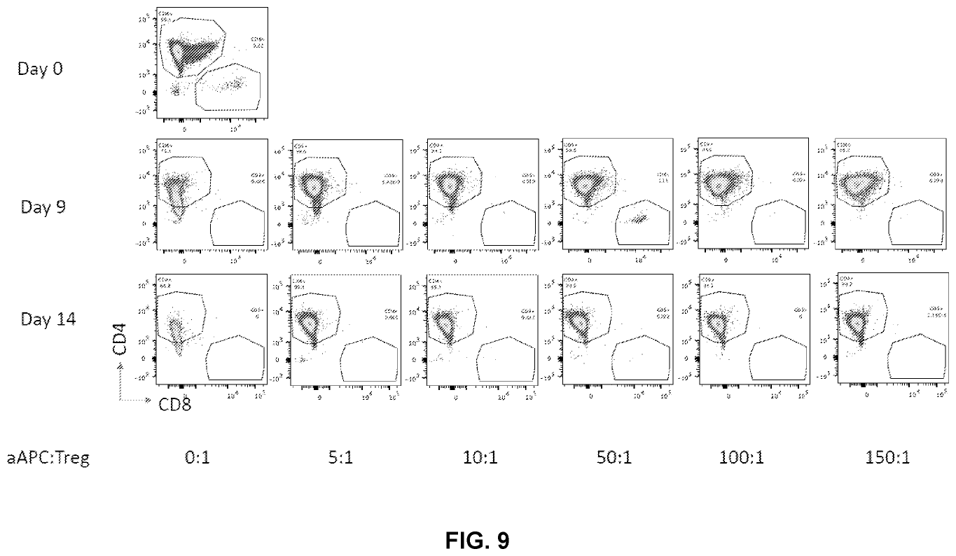

FIG. 9 shows CD4+/CD3+ cells at day 0, 9, and 14 with aAPC:Treg at 0:1, 5:1, 10:1, 50:1, 100:1, and 150:1.

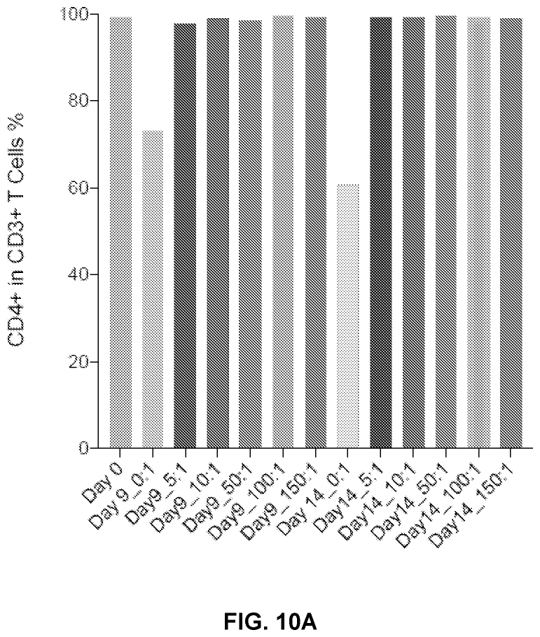

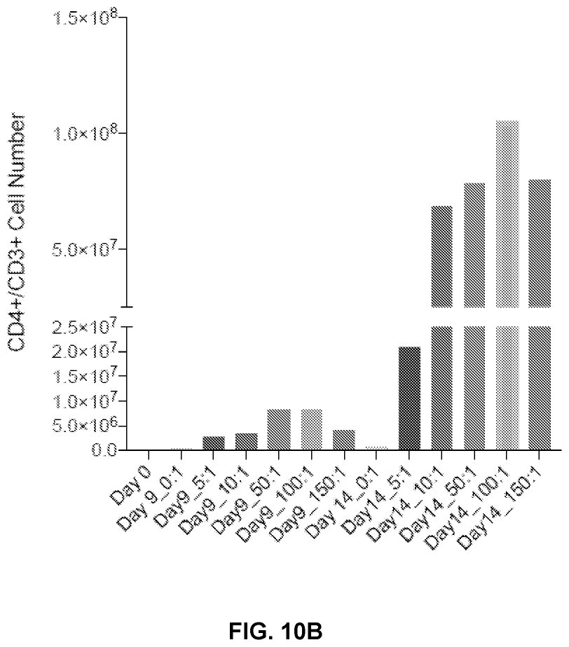

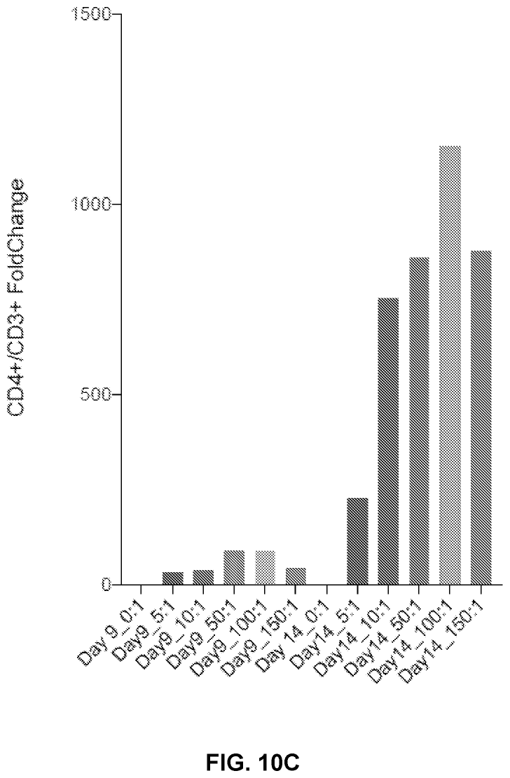

FIGS. 10A to 10C show CD4+ percentage in CD3+ T cells (FIG. 10A), CD4+/CD3+ cell number (FIG. 10B), and CD4+/CD3+ fold change (FIG. 10C) at day 0, 9, and 14 with aAPC:Treg at 0:1, 5:1, 10:1, 50:1, 100:1, and 150:1.

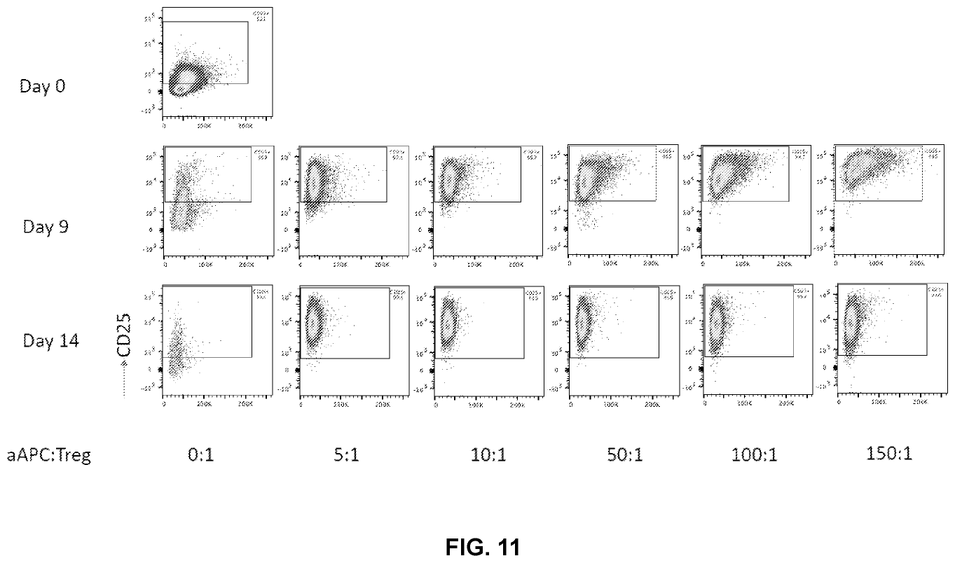

FIG. 11 shows CD25+/CD4+ cells at day 0, 9, and 14 with aAPC:Treg at 0:1, 5:1, 10:1, 50:1, 100:1, and 150:1.

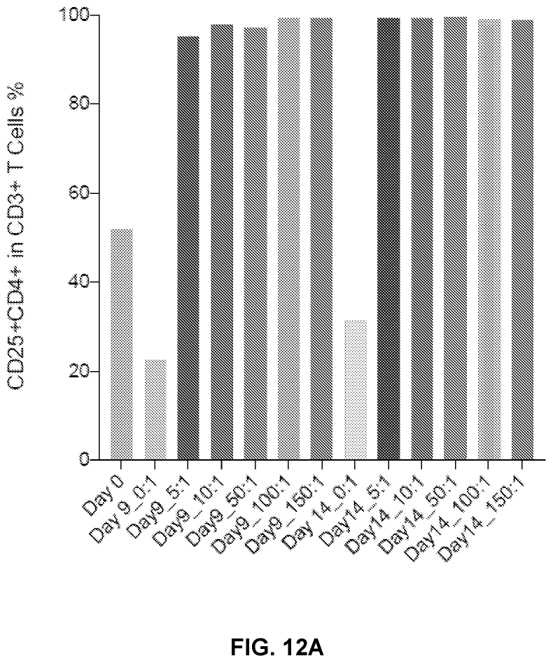

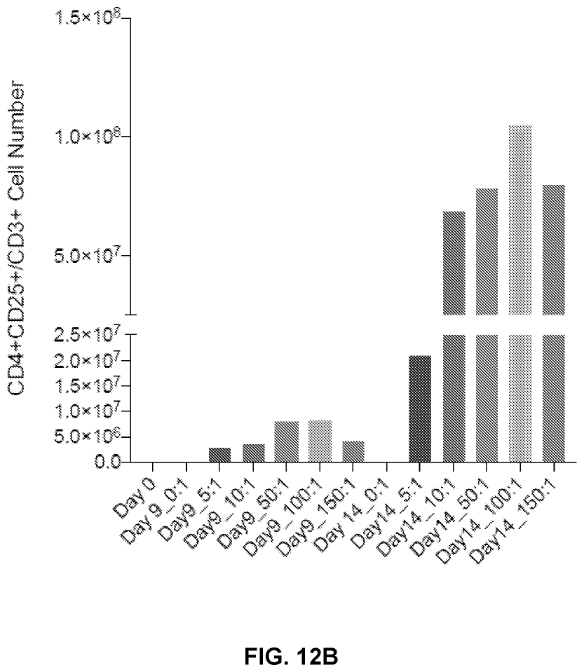

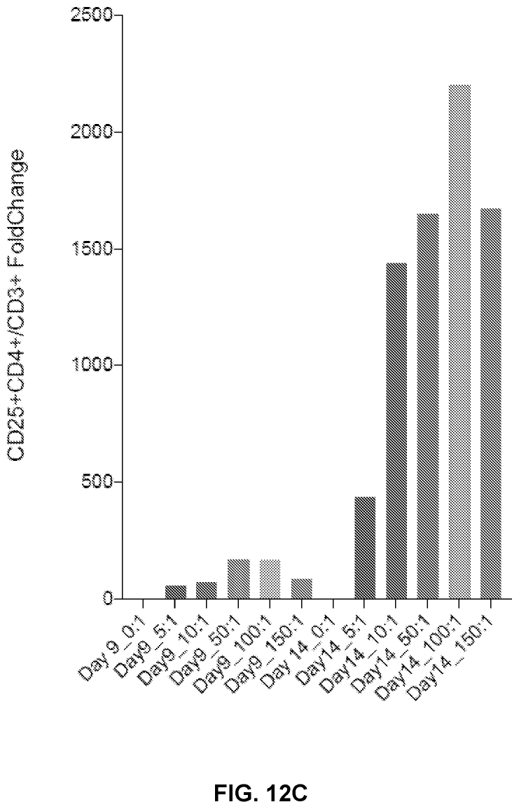

FIGS. 12A to 12C show CD25+CD4+ percentage in CD3+ T cells (FIG. 12A), CD25+CD4+/CD3+ cell number (FIG. 12B), and CD25+CD4+/CD3+ fold change (FIG. 12C) at day 0, 9, and 14 with aAPC:Treg at 0:1, 5:1, 10:1, 50:1, 100:1, and 150:1.

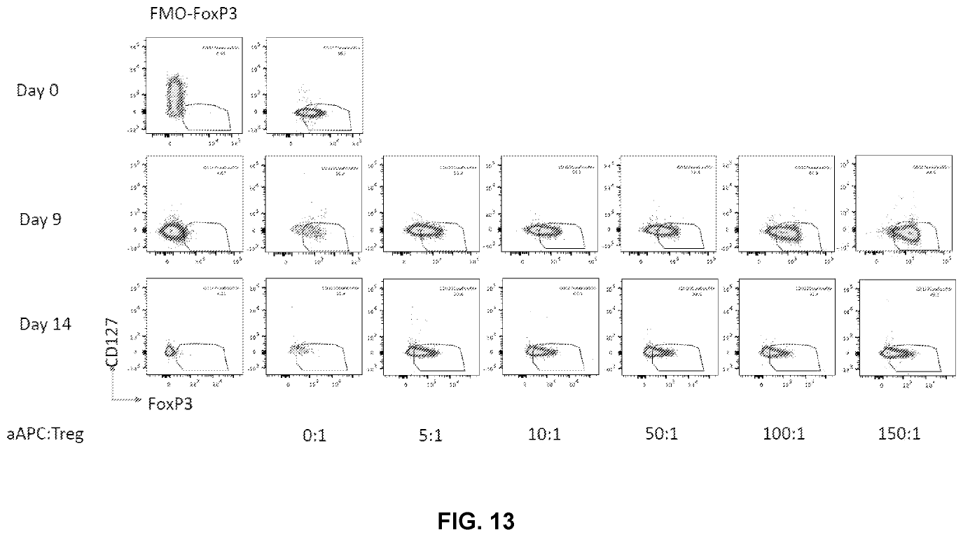

FIG. 13 shows CD25+CD4+CD127lowFoxP3/CD3+ cells at day 0, 9, and 14 with aAPC:Treg at 0:1, 5:1, 10:1, 50:1, 100:1, and 150:1.

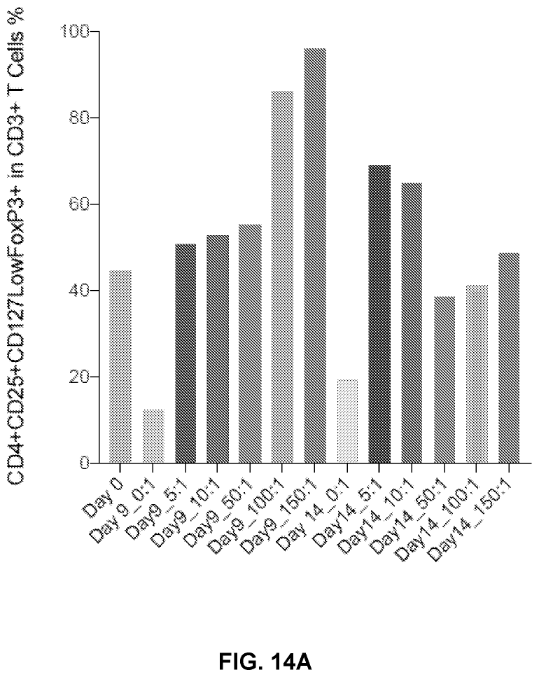

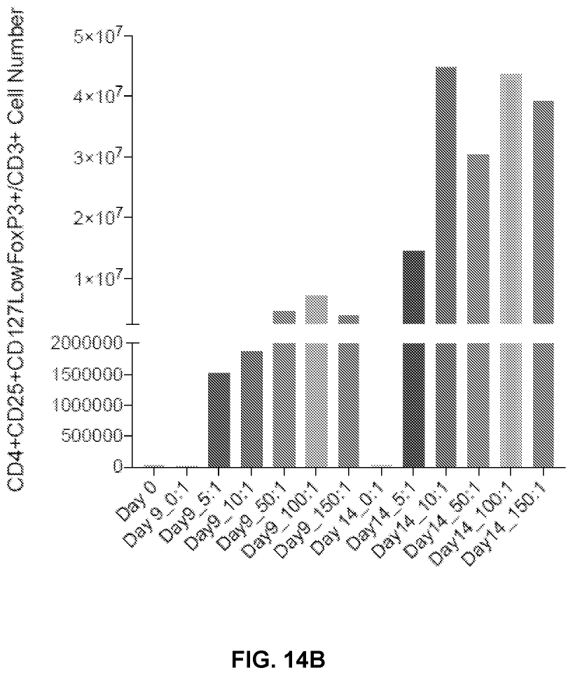

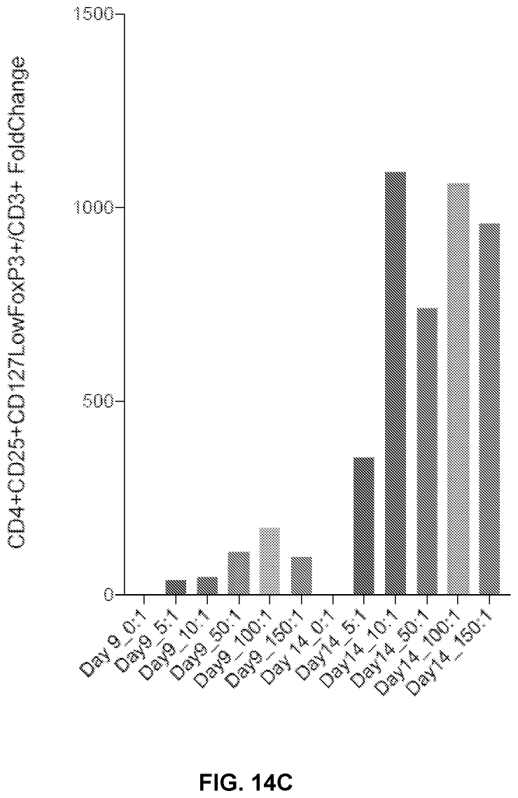

FIGS. 14A to 14C show CD25+CD4+CD127lowFoxP3 percentage in CD3+ T cells (FIG. 14A), CD25+CD4+CD127lowFoxP3/CD3+ cell number (FIG. 14B), and CD25+CD4+CD127lowFoxP3/CD3+ fold change (FIG. 14C) at day 0, 9, and 14 with aAPC:Treg at 0:1, 5:1, 10:1, 50:1, 100:1, and 150:1.

### Example 5: Biomarker Data for Human Treg Expansion with Quadruplet aAPCs

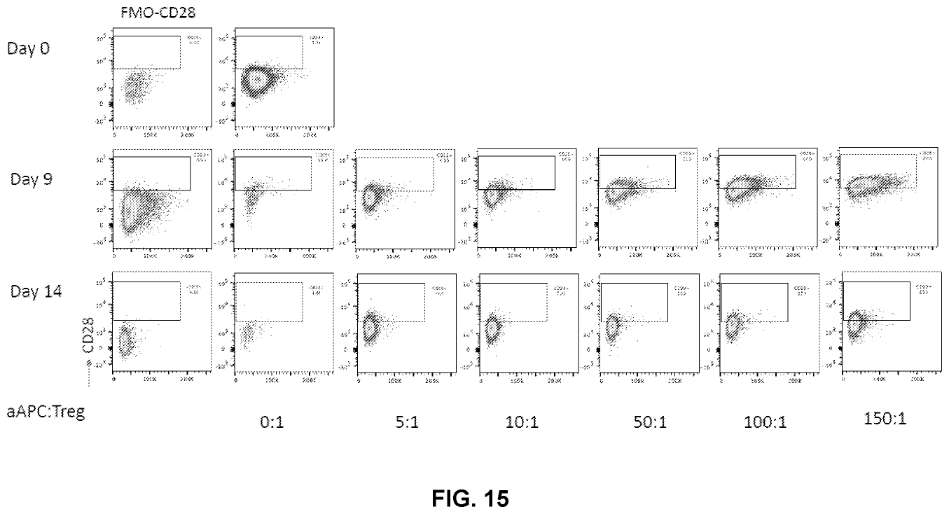

FIG. 15 shows CD28+ in CD127lowFoxP3+ cells at day 0, 9, and 14 with aAPC:Treg at 0:1, 5:1, 10:1, 50:1, 100:1, and 150:1.

FIGS. 16A to 16C show CD28+ percentage in CD127lowFoxP3+ T cells (FIG. 16A), CD28+/CD127lowFoxP3+ cell number (FIG. 16B), and CD28+/CD127lowFoxP3+ fold change (FIG. 16C) at day 0, 9, and 14 with aAPC:Treg at 0:1, 5:1, 10:1, 50:1, 100:1, and 150:1.

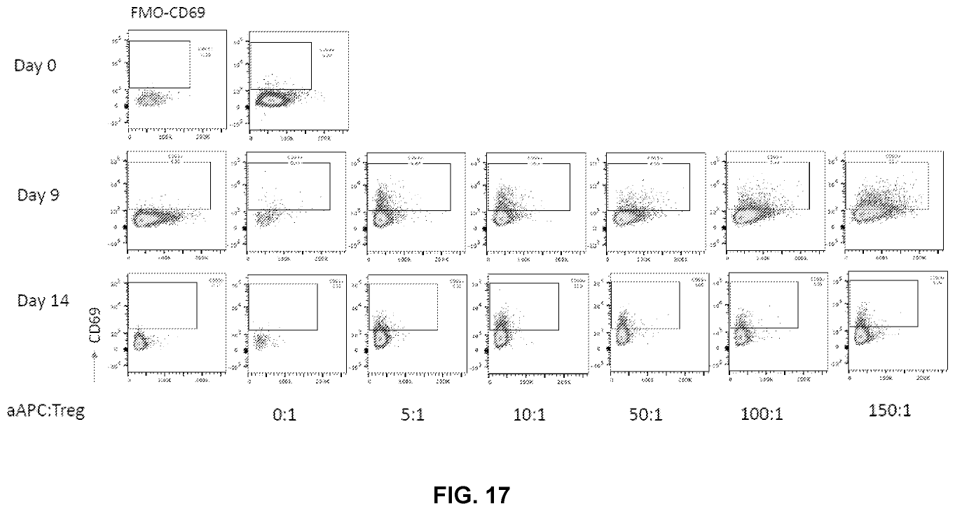

FIG. 17 shows CD69+/CD127lowFoxP3+ cells at day 0, 9, and 14 with aAPC:Treg at 0:1, 5:1, 10:1, 50:1, 100:1, and 150:1.

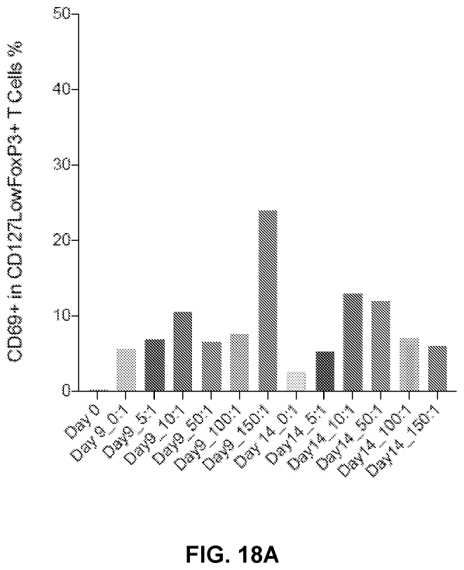

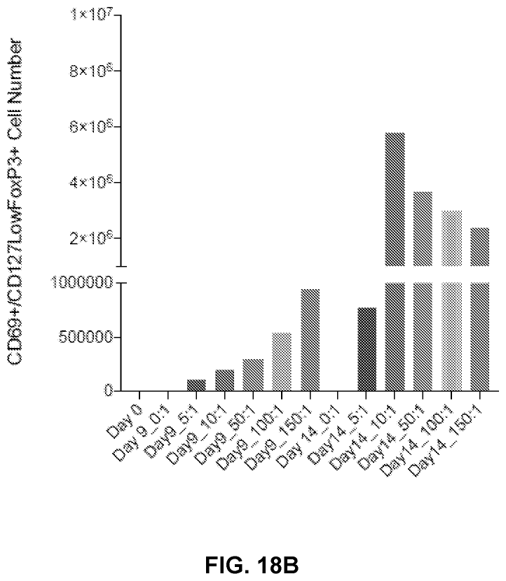

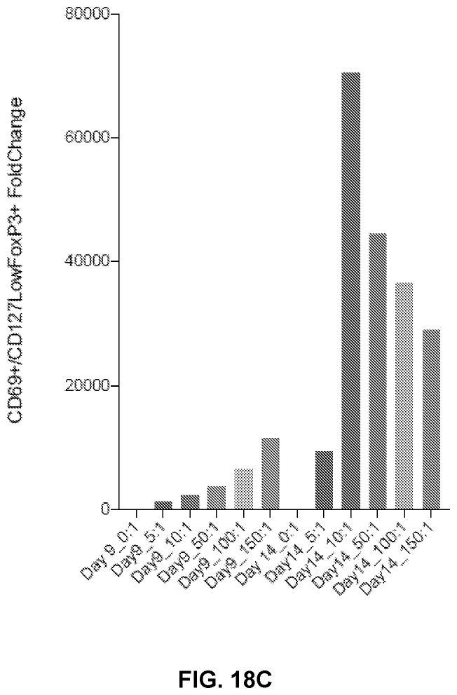

FIGS. 18A to 18C show CD69+ percentage in CD127lowFoxP3+ T cells (FIG. 18A), CD69+/CD127lowFoxP3+ cell number (FIG. 18B), and CD69+/CD127lowFoxP3+ fold change (FIG. 18C) at day 0, 9, and 14 with aAPC:Treg at 0:1, 5:1, 10:1, 50:1, 100:1, and 150:1.

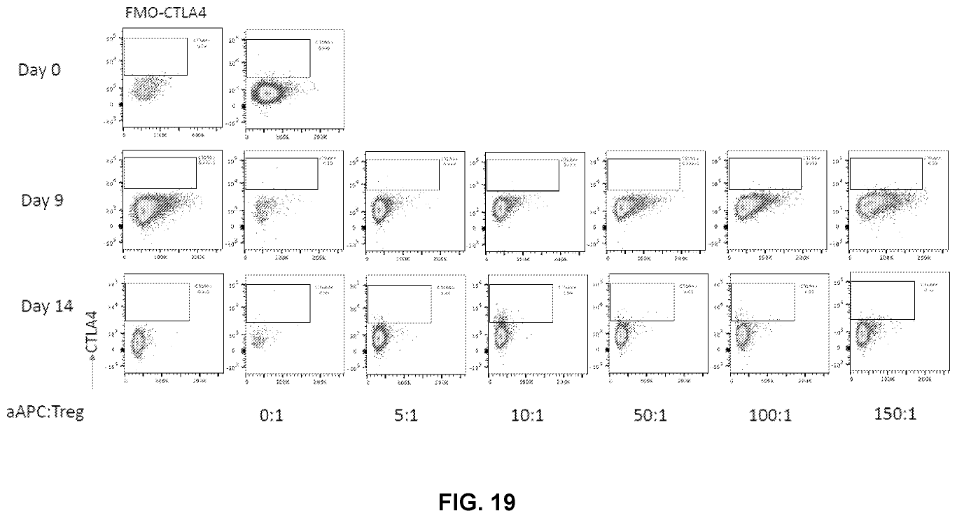

FIG. 19 shows CTLA4+/CD127lowFoxP3+ cells at day 0, 9, and 14 with aAPC:Treg at 0:1, 5:1, 10:1, 50:1, 100:1, and 150:1.

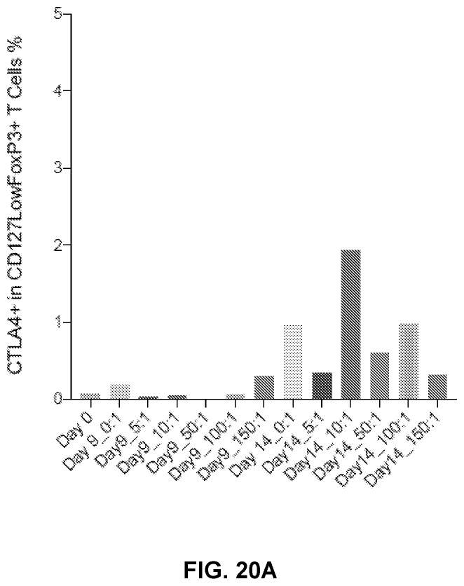

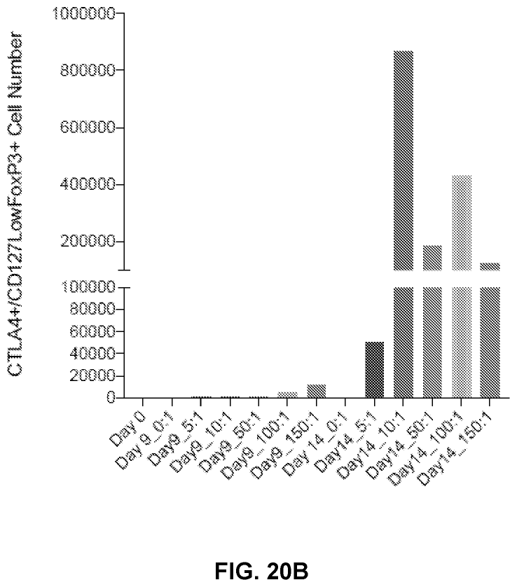

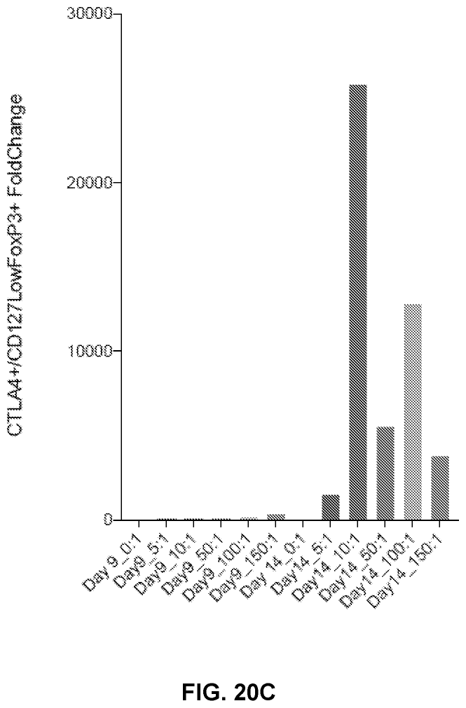

FIGS. 20A to 20C show CTLA4+ percentage in CD127lowFoxP3+ T cells (FIG. 20A), CTLA4+/CD127lowFoxP3+ cell number (FIG. 20B), and CTLA4+/CD127lowFoxP3+ fold change (FIG. 20C) at day 0, 9, and 14 with aAPC:Treg at 0:1, 5:1, 10:1, 50:1, 100:1, and 150:1.

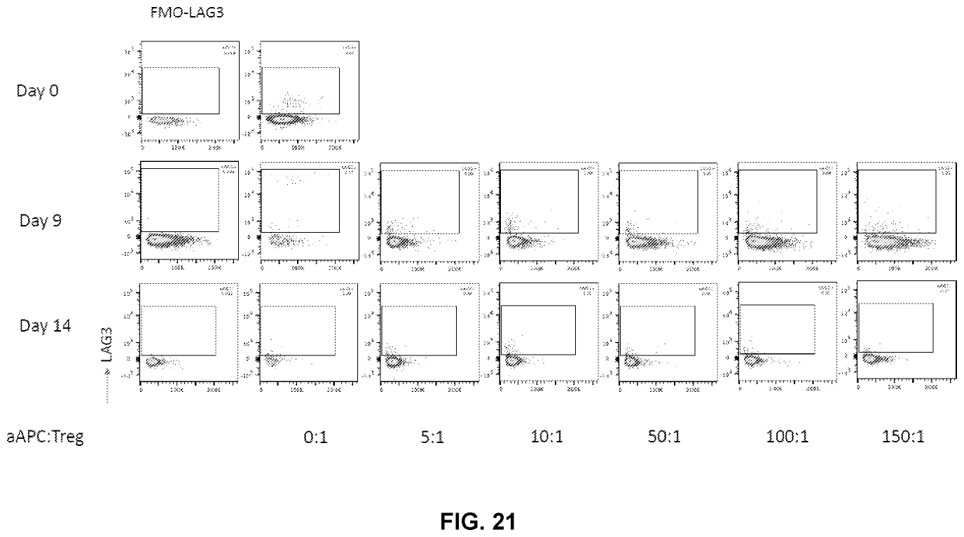

FIG. 21 shows LAG3+/CD127lowFoxP3+ cells at day 0, 9, and 14 with aAPC:Treg at 0:1, 5:1, 10:1, 50:1, 100:1, and 150:1.

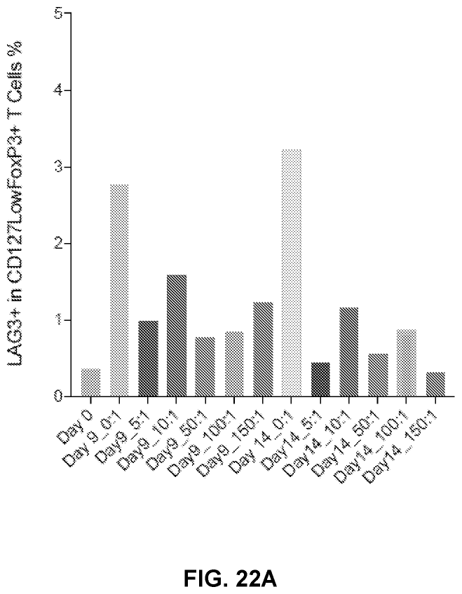

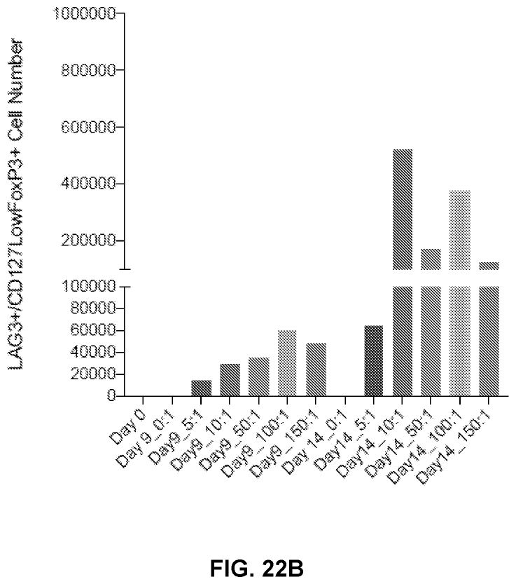

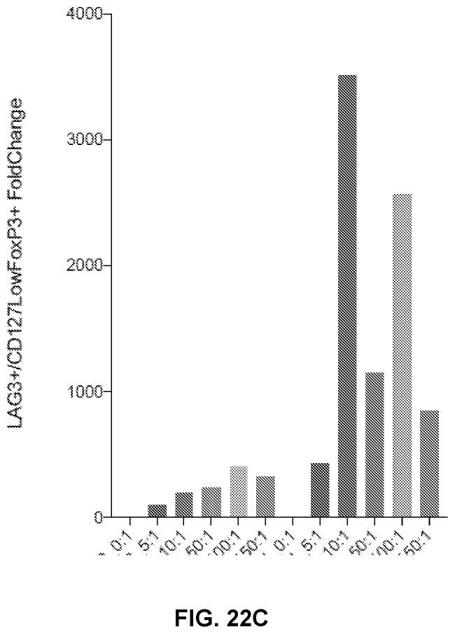

FIGS. 22A to 22C show LAG3+ percentage in CD127lowFoxP3+ T cells (FIG. 22A), LAG3+/CD127lowFoxP3+ cell number (FIG. 22B), and LAG3+/CD127lowFoxP3+ fold change (FIG. 22C) at day 0, 9, and 14 with aAPC:Treg at 0:1, 5:1, 10:1, 50:1, 100:1, and 150:1.

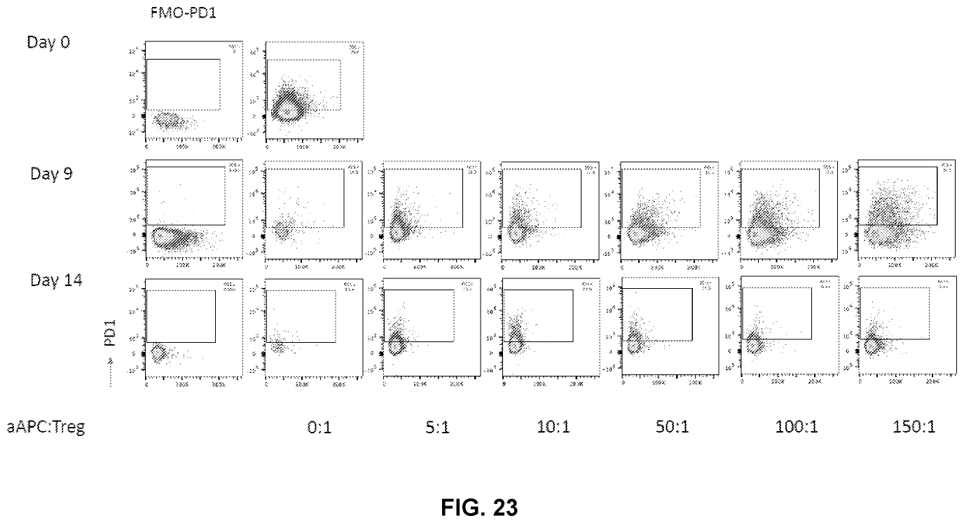

FIG. 23 shows PD1+/CD127lowFoxP3+ cells at day 0, 9, and 14 with aAPC:Treg at 0:1, 5:1, 10:1, 50:1, 100:1, and 150:1.

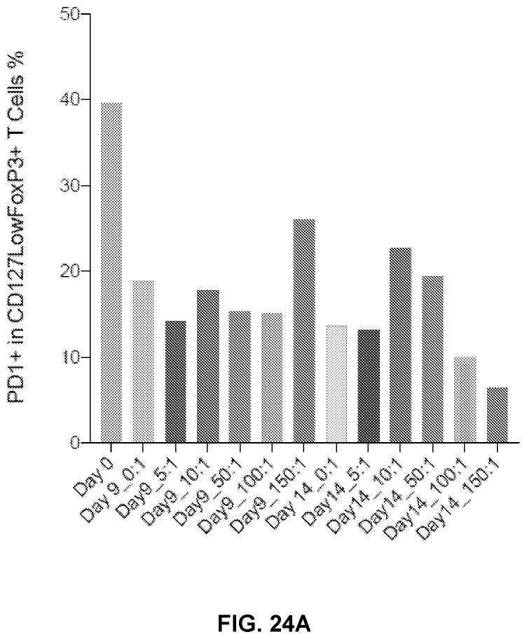

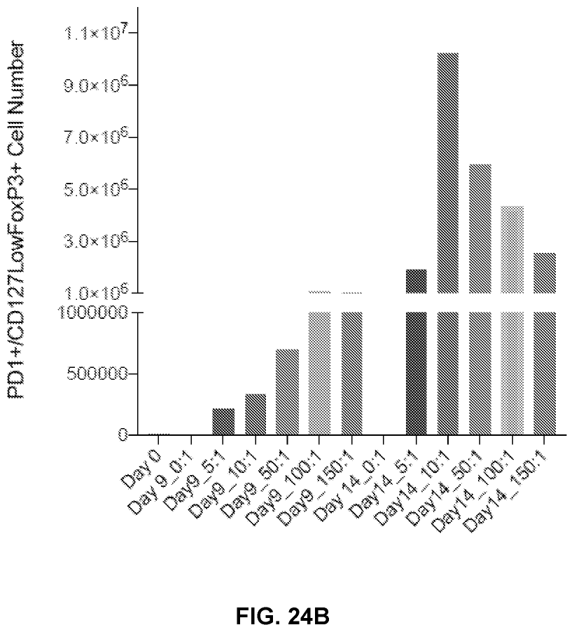

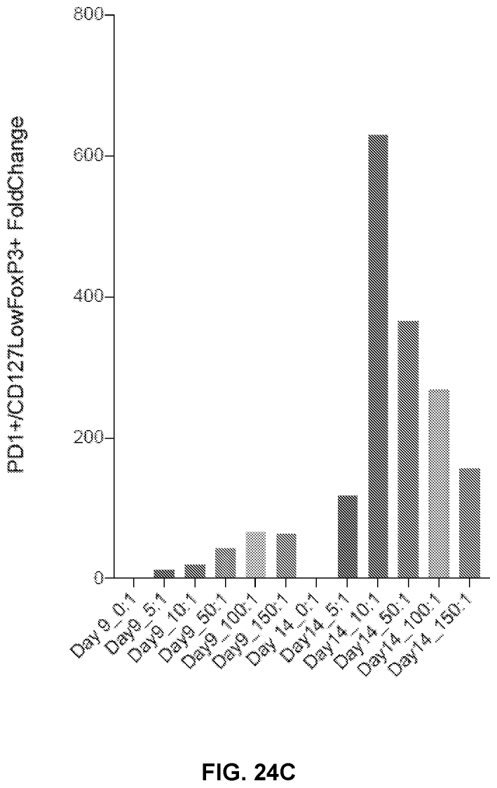

FIGS. 24A to 24C show PD1+ percentage in CD127lowFoxP3+ T cells (FIG. 24A), PD1+/CD127lowFoxP3+ cell number (FIG. 24B), and PD1+/CD127lowFoxP3+ fold change (FIG. 24C) at day 0, 9, and 14 with aAPC:Treg at 0:1, 5:1, 10:1, 50:1, 100:1, and 150:1.

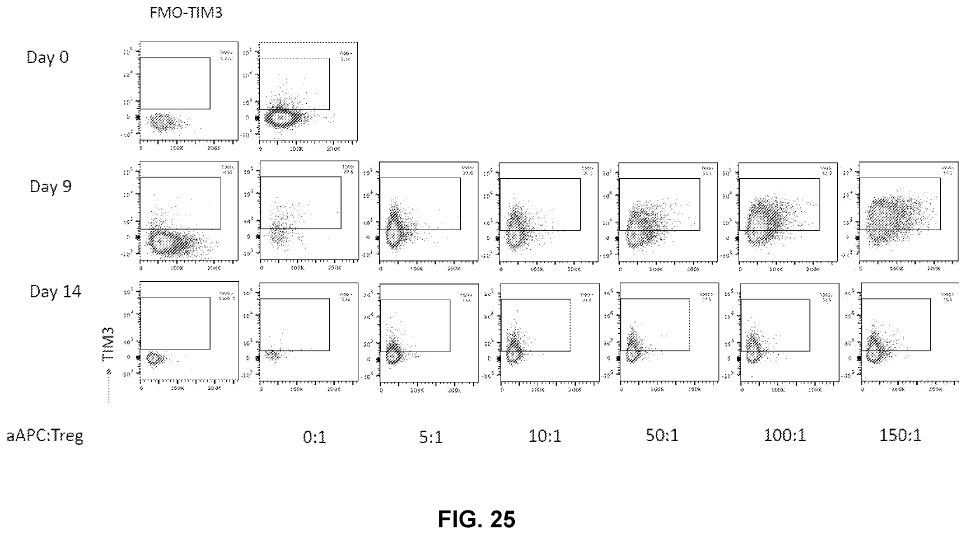

FIG. 25 shows TIM3+/CD127lowFoxP3+ cells at day 0, 9, and 14 with aAPC:Treg at 0:1, 5:1, 10:1, 50:1, 100:1, and 150:1.

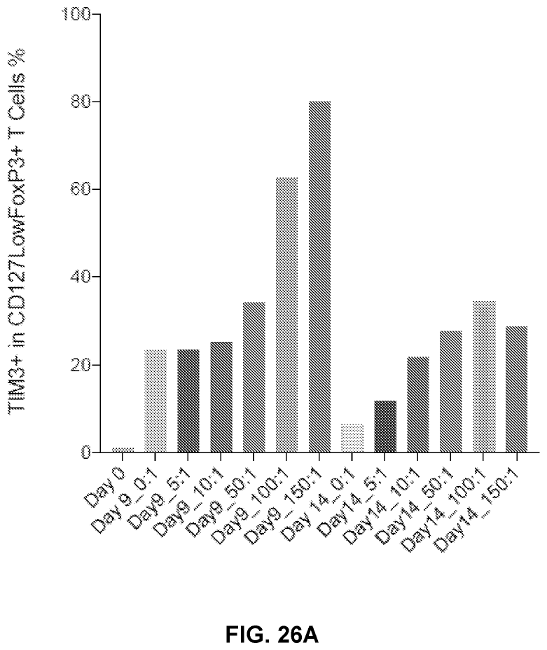

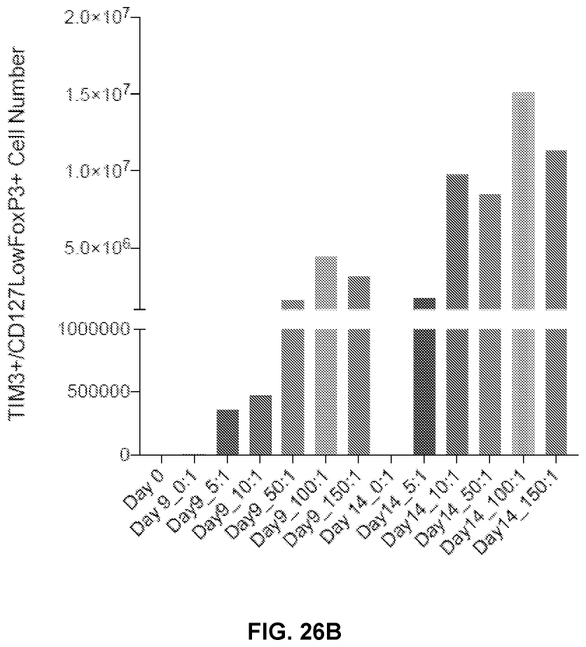

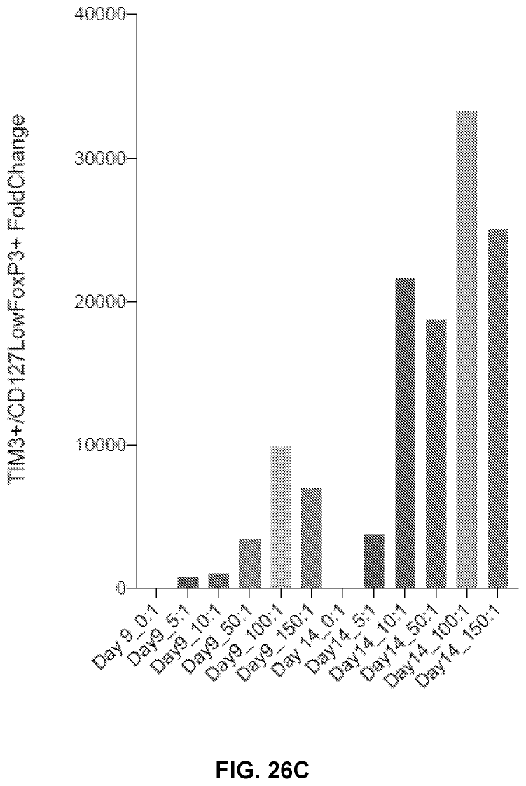

FIGS. 26A to 26C show TIM3+ percentage in CD127lowFoxP3+ T cells (FIG. 26A), TIM3+/CD127lowFoxP3+ cell number (FIG. 26B), and TIM3+/CD127lowFoxP3+ fold change (FIG. 26C) at day 0, 9, and 14 with aAPC:Treg at 0:1, 5:1, 10:1, 50:1, 100:1, and 150:1.

### Example 6: Human Healthy Donor Tregs and Biomarkers Expansion Data with Quadruplet aAPCs

Experimental Design

0.8×10⁶ live CD4+CD25+CD127low from Stemcell Technologies were planted with 8×10⁶ irradiated quadruplet aAPCs co-cultured with 5% human AB serum and 30 IU/ml human IL2 for 14 days. CD4, CD25, CD127low and FoxP3 were phenytypes and were positive in all defined as Tregs. Activation marker CD28, CD69, and inhibitory marker CTLA4, LAG3, PD1, and TIM3 were evaluated on day 0, day 11, and day 14.

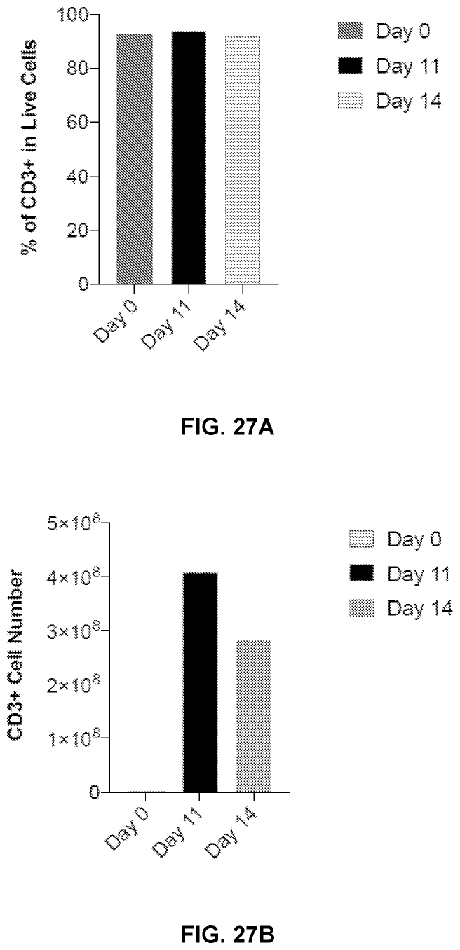

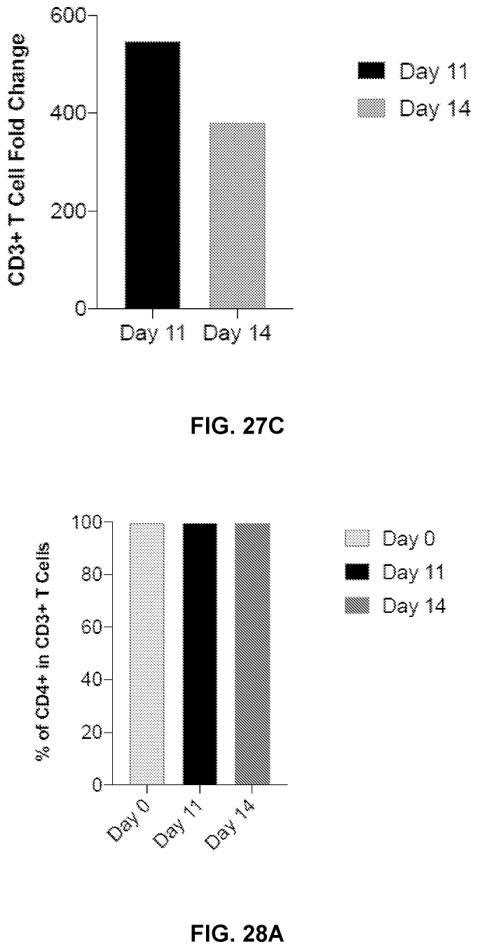

FIGS. 27A to 27C show CD3 percentage (FIG. 27A), number (FIG. 278B), and fold change (FIG. 27C) in live cells at day 0, 11, and 14.

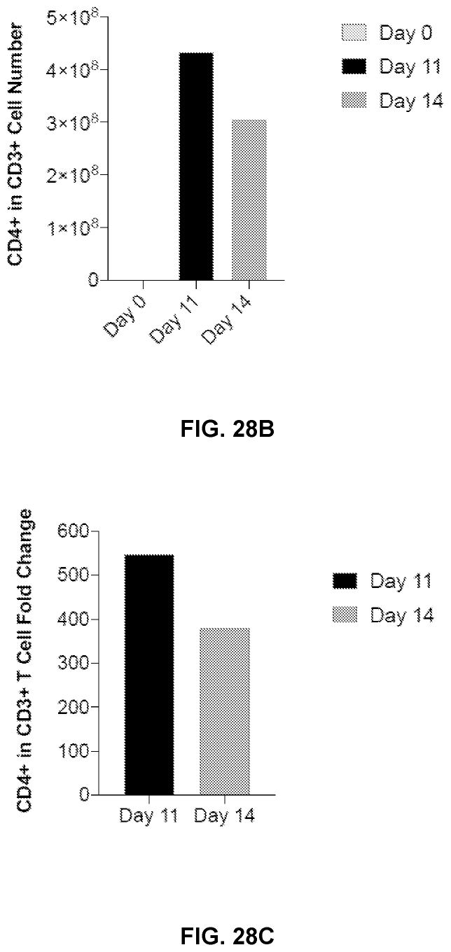

FIGS. 28A to 28C show CD4+ percentage in CD3+ T cells (FIG. 28A), CD4+/CD3+ cell number (FIG. 28B), and CD4+/CD3+ fold change (FIG. 28C) at day 0, 11, and 14.

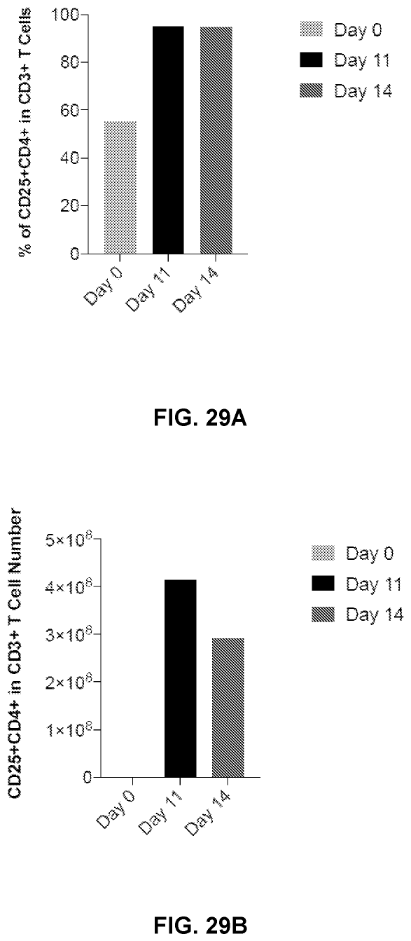

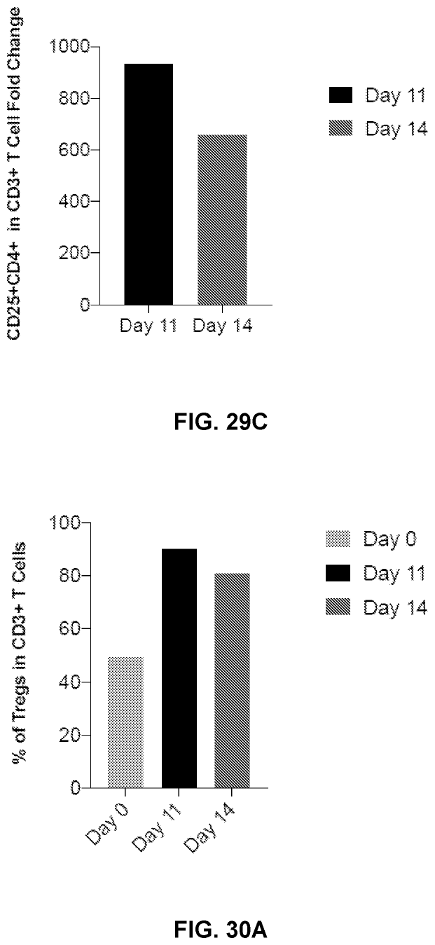

FIGS. 29A to 29C show CD25+CD4+ percentage in CD3+ T cells (FIG. 29A), CD25+CD4+/CD3+ cell number (FIG. 29B), and CD25+CD4+/CD3+ fold change (FIG. 29C) at day 0, 11, and 14.

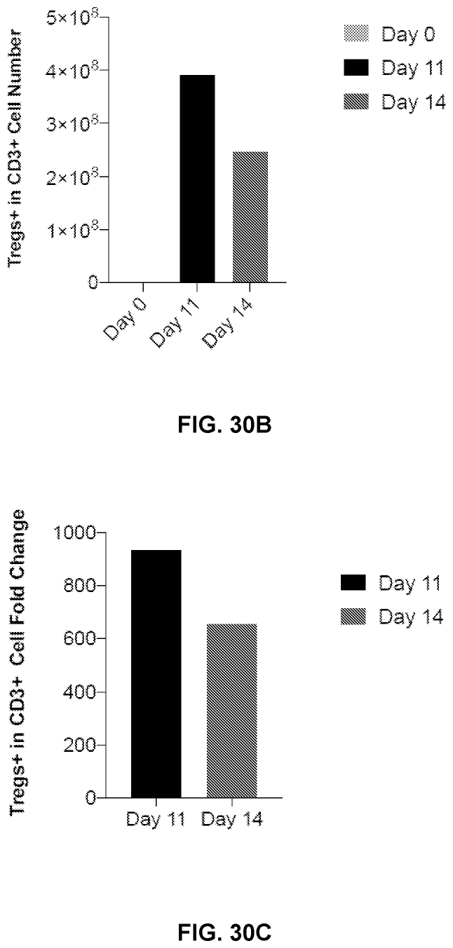

FIGS. 30A to 30C show CD25+CD4+CD127lowFoxP3 percentage in CD3+ T cells (FIG. 30A), CD25+CD4+CD127lowFoxP3/CD3+ cell number (FIG. 30B), and CD25+CD4+CD127lowFoxP3/CD3+ fold change (FIG. 30C) at day 0, 11, and 14.

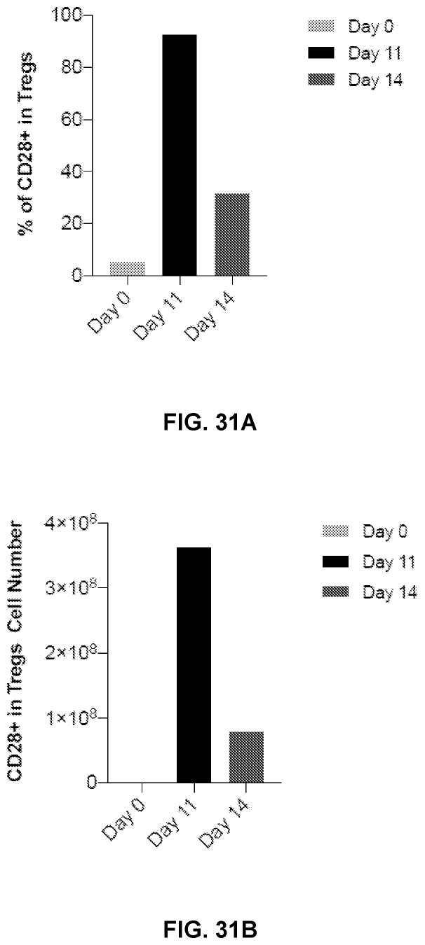

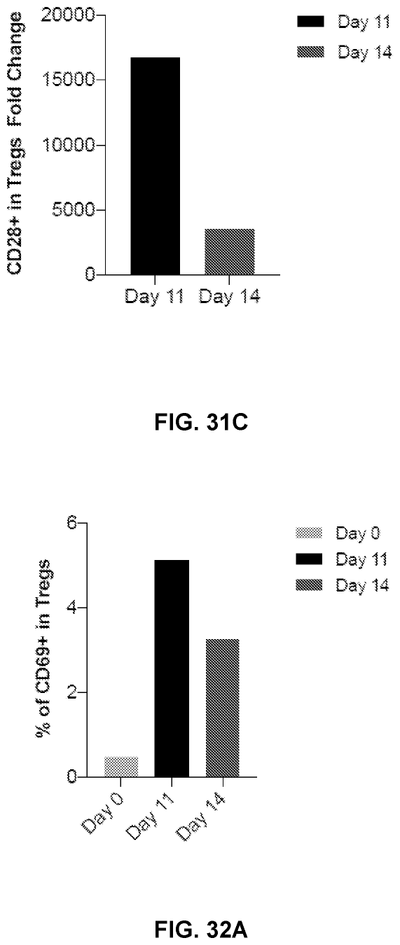

FIGS. 31A to 31C show CD28+ percentage in CD127lowFoxP3+ T cells (FIG. 31A), CD28+CD127lowFoxP3+ cell number (FIG. 31B), and CD28+CD127lowFoxP3+ fold change (FIG. 31C) at day 0, 11, and 14.

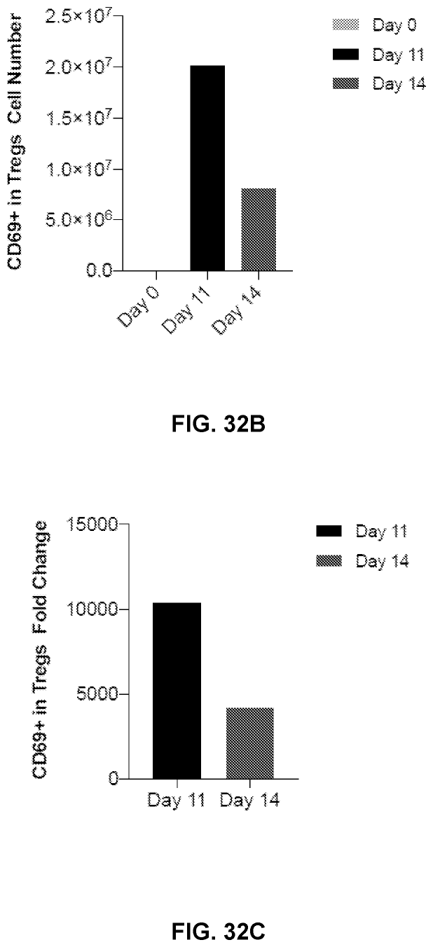

FIGS. 32A to 32C show CD69+ percentage in CD127lowFoxP3+ T cells (FIG. 32A), CD28+CD127lowFoxP3+ cell number (FIG. 32B), and CD28+CD127lowFoxP3+ fold change (FIG. 32C) at day 0, 11, and 14.

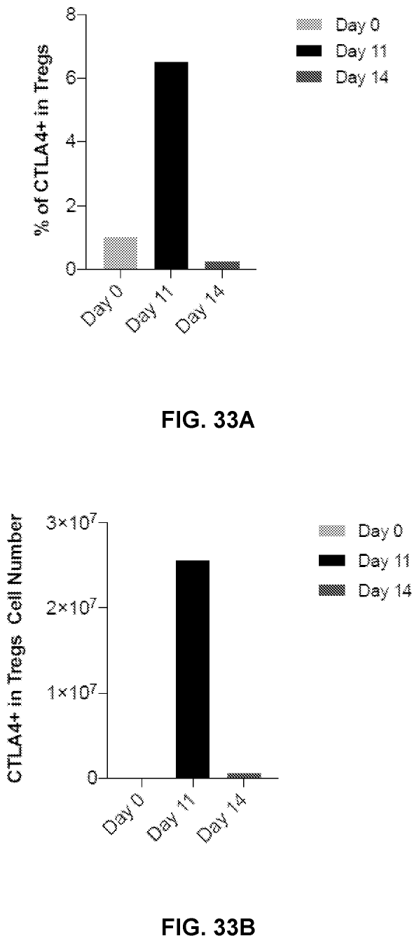

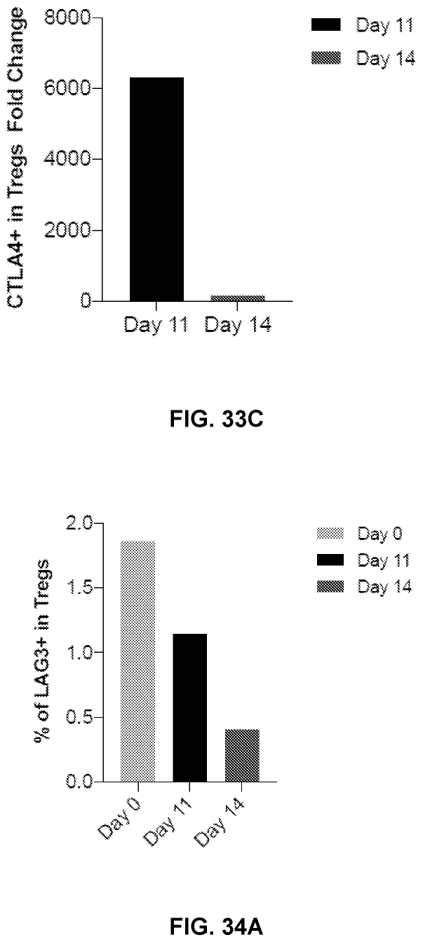

FIGS. 33A to 33C show CTLA4+ percentage in CD127lowFoxP3+ T cells (FIG. 33A), CTLA4+CD127lowFoxP3+ cell number (FIG. 33B), and CTLA4+CD127lowFoxP3+ fold change (FIG. 33C) at day 0, 11, and 14.

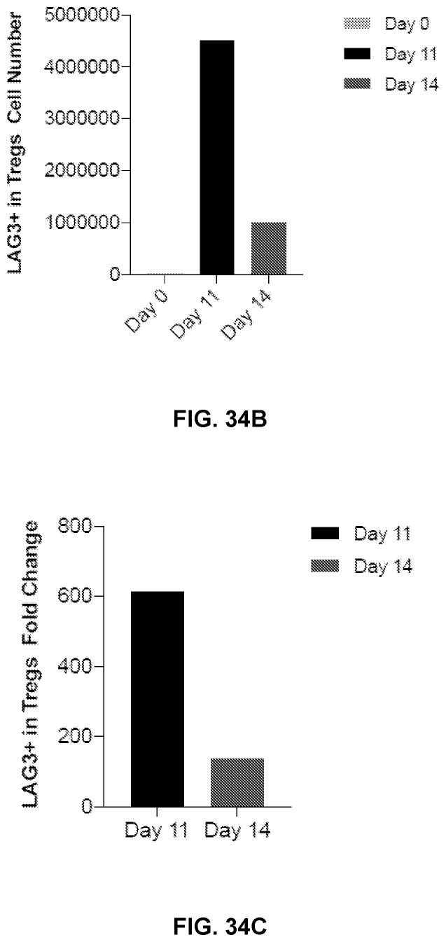

FIGS. 34A to 34C show LAG3+ percentage in CD127lowFoxP3+ T cells (FIG. 34A), LAG3+CD127lowFoxP3+ cell number (FIG. 34B), and LAG3+CD127lowFoxP3+ fold change (FIG. 34C) at day 0, 11, and 14.

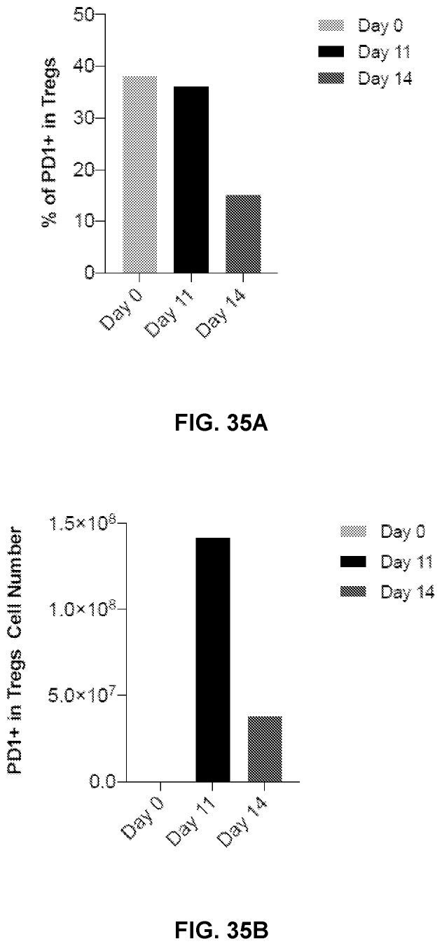

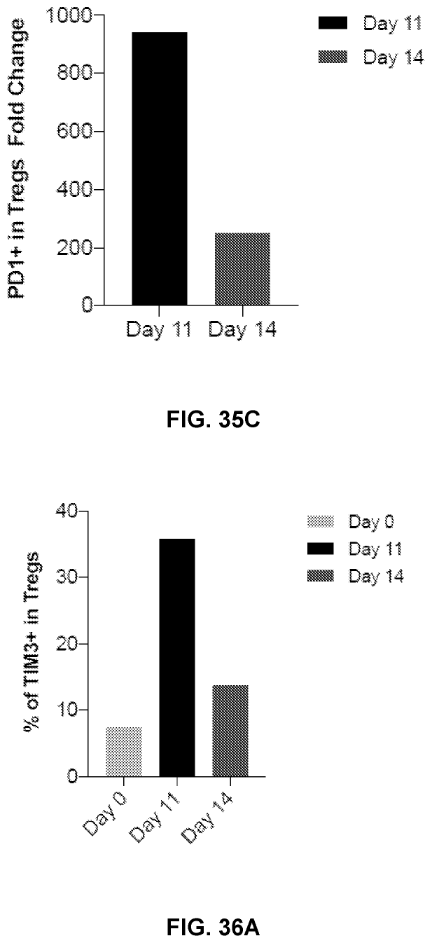

FIGS. 35A to 35C show PD1+ percentage in CD127lowFoxP3+ T cells (FIG. 35A), PD1+CD127lowFoxP3+ cell number (FIG. 35B), and PD1+CD127lowFoxP3+ fold change (FIG. 35C) at day 0, 11, and 14.

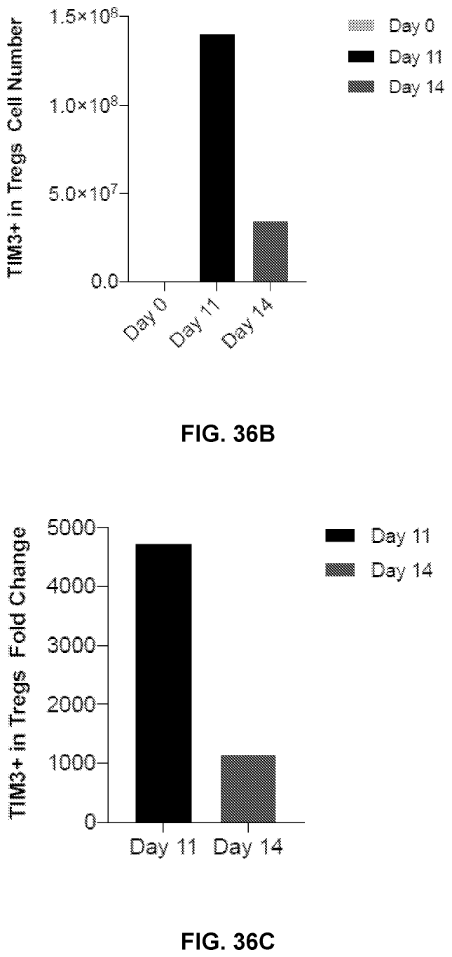

FIGS. 36A to 36C show TIM3+ percentage in CD127lowFoxP3+ T cells (FIG. 36A), TIM3+CD127lowFoxP3+ cell number (FIG. 6B), and TIM3+CD127lowFoxP3+ fold change (FIG. 36C) at day 0, 11, and 14.

### CONCLUSION

CD4+CD25+ started at 50% and bounced up to 99% at day 11 and day 14. Tregs were started from 50% and increased to 90% at day 11 and decreased to 80% at day 14. Tregs had more than 930 fold increase by day 11 but decreased to 650 fold at day 14.

CD28 and CD69 had huge fold increase at day 11 that were more than 10,000 fold.

CTLA4, TIM3, PD1, and LAG3 also had decent fold increase at day 11, specifically CTLA4, that was going to 6,000 fold but dropped to 130 fold by day 14.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for producing regulatory T (T_{reg}) cells, comprising
(a) isolating PBMCs from a donor;
(b) isolating T cells from the PBMCs; and
(c) contacting the T cells with artificial antigen presenting cells (AAPCs) expressing on its outer membrane an anti-CD3 scFv, an anti-CD28 scFv, a 41 BB ligand (41 BBL), an IL15 receptor (IL15R), and optionally a heparin-binding domain (HBD) to produce activated T_{reg} cells.

2. The method of claim 1, wherein the AAPCs comprises an antigen presenting cell line.

3. The method of claim 2, wherein the antigen presenting cell line is selected from the group consisting of a K562, a NIH/3T3, a Chinese hamster ovary (CHO), or a Human Embryonic Kidney (HEK) cell line.

4. The method of claim 1, further comprising transducing the activated Treg cells with a viral vector encoding a CAR polypeptide.

5. The method of claim 4, wherein the CAR polypeptide comprises a CD83 antigen binding domain, a transmembrane domain, an intracellular signaling domain, and a co-stimulatory signaling region.

6. The method of claim 5, wherein the CD83 antigen binding domain is a single-chain variable fragment (scFv) of an antibody that specifically binds CD83.

7. The method of claim 6, wherein the anti-CD83 scFv comprises a variable heavy (V_{H}) domain having CDR1, CDR2 and CDR3 sequences and a variable light (V_{L}) domain having CDR1, CDR2 and CDR3 sequences, wherein the CDR1 sequence of the V_{H} domain comprises the amino acid sequence SEQ ID NO:1, SEQ ID NO:7, or SEQ ID NO:13; the CDR2 sequence of the V_{H} domain comprises the amino acid sequence SEQ ID NO:2, SEQ ID NO:8, or SEQ ID NO:14; the CDR3 sequence of the V_{H} domain comprises the amino acid sequence SEQ ID NO:3, SEQ ID NO:9, or SEQ ID NO:15; the CDR1 sequence of the V_{L} comprises the amino acid sequence SEQ ID NO:4, SEQ ID NO:10, or SEQ ID NO:16; the CDR2 sequence of the V_{L} domain comprises the amino acid sequence SEQ ID NO:5, SEQ ID NO:11, or SEQ ID NO:17; and the CDR3 sequence of the V_{L} domain comprises the amino acid sequence SEQ ID NO:6, SEQ ID NO:12, or SEQ ID NO:18.

8. The method of claim 7, wherein the anti-CD83 scFv V_{H} domain comprises the amino acid sequence SEQ ID NO:19, SEQ ID NO:48, SEQ ID NO:49, SEQ ID NO:50, SEQ ID NO:51, SEQ ID NO:52, or SEQ ID NO:53.

9. The method of claim 7, wherein the anti-CD83 scFv V_{L} domain comprises the amino acid sequence SEQ ID NO:20, SEQ ID NO:54, or SEQ ID NO:55.

10. The method of claim 6, wherein the anti-CD83 scFv comprises the amino acid sequence SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, or SEQ ID NO:71.

11. The method of claim 5, wherein the costimulatory signaling region comprises the cytoplasmic domain of a costimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and any combination thereof

12. The method of claim 4, wherein the CAR polypeptide is defined by the formula:
SP-CD83-HG-TM-CSR-ISD; or
SP-CD83-HG-TM-ISD-CSR
wherein “SP” represents a signal peptide,
wherein “CD83” represents a CD83-binding region,
wherein “HG” represents and optional hinge domain,
wherein “TM” represents a transmembrane domain,
wherein “CSR” represents a co-stimulatory signaling region,
wherein “ISD” represents an intracellular signaling domain, and
wherein “-” represents a bivalent linker.

13. The method of claim 5, wherein the intracellular signaling domain comprises a CD3 zeta (CD3ζ) signaling domain.

14. A method for producing regulatory CAR-T (CAR-T_{reg}) cells, comprising
(d) isolating PBMCs from a donor;
(e) isolating T cells from the PBMCs;
(f) stimulating the T cells with CD3/CD28 beads;
(g) transducing the activated T cells with a viral vector encoding a CAR polypeptide; and
(h) transducing the activated T cells with a viral vector encoding IL2RB, FOXP3, SOCS, or any combination thereof.

15-26. (canceled)


SEQUENCE LISTING (87 sequences)

SEQ ID NO: 1 - protein, 12 aa, Artificial Sequence
  Synthetic Construct
GFSITTGGYWWT

SEQ ID NO: 2 - protein, 17 aa, Artificial Sequence
  Synthetic Construct
GYIFSSGNTNYNPSIKS

SEQ ID NO: 3 - protein, 12 aa, Artificial Sequence
  Synthetic Construct
CARAYGKLGFDY

SEQ ID NO: 4 - protein, 12 aa, Artificial Sequence
  Synthetic Construct
TLSSQHSTYTIG

SEQ ID NO: 5 - protein, 11 aa, Artificial Sequence
  Synthetic Construct
VNSDGSHSKGD

SEQ ID NO: 6 - protein, 9 aa, Artificial Sequence
  Synthetic Construct
GSSDSSGYV

SEQ ID NO: 7 - protein, 5 aa, Artificial Sequence
  Synthetic Construct
SDGIS

SEQ ID NO: 8 - protein, 16 aa, Artificial Sequence
  Synthetic Construct
IISSGGNTYYASWAKG

SEQ ID NO: 9 - protein, 8 aa, Artificial Sequence
  Synthetic Construct
VVGGTYSI

SEQ ID NO: 10 - protein, 13 aa, Artificial Sequence
  Synthetic Construct
QSSQSVYNNDFLS

SEQ ID NO: 11 - protein, 7 aa, Artificial Sequence
  Synthetic Construct
YASTLAS

SEQ ID NO: 12 - protein, 13 aa, Artificial Sequence
  Synthetic Construct
TGTYGNSAWYEDA

SEQ ID NO: 13 - protein, 5 aa, Artificial Sequence
  Synthetic Construct
SNAMI

SEQ ID NO: 14 - protein, 16 aa, Artificial Sequence
  Synthetic Construct
AMDSNSRTYYATWAKG

SEQ ID NO: 15 - protein, 11 aa, Artificial Sequence
  Synthetic Construct
GDGGSSDYTEM

SEQ ID NO: 16 - protein, 13 aa, Artificial Sequence
  Synthetic Construct
QSSQSVYGNNELS

SEQ ID NO: 17 - protein, 7 aa, Artificial Sequence
  Synthetic Construct
QASSLAS

SEQ ID NO: 18 - protein, 11 aa, Artificial Sequence
  Synthetic Construct
LGEYSISADNH

SEQ ID NO: 19 - protein, 120 aa, Artificial Sequence
  Synthetic Construct
QVQLKESGPGLVKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTLVTVSS

SEQ ID NO: 20 - protein, 111 aa, Artificial Sequence
  Synthetic Construct
QPVLTQSPSASASLGNSVKITCTLSSQHSTYTIGWYQQHPDKAPKYVMYVNSDGSHSKGDGIPDRFSGSSSGAHRYLSISNIQPEDEADYFCGSSDSSGYVFGSGTQLTVL

SEQ ID NO: 21 - protein, 454 aa, Artificial Sequence
  Synthetic Construct
METGLRWLLLVAVLKGVQCQSVEESGGRLVTPGTPLTLTCTVSGFSLSNNAINWVRQAPGKGLEWIGYIWSGGLTYYANWAEGRFTISKTSTTVDLKMTSPTIEDTATYFCARGINNSALWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 22 - protein, 239 aa, Artificial Sequence
  Synthetic Construct
MDMRAPTQLLGLLLLWLPGARCADVVMTQTPASVSAAVGGTVTINCQASESISNYLSWYQQKPGQPPKLLIYRTSTLASGVSSRFKGSGSGTEYTLTISGVQCDDVATYYCQCTSGGKFISDGAAFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 23 - protein, 452 aa, Artificial Sequence
  Synthetic Construct
METGLRWLLLVAVLKGVQCQSVEESGGRLVTPGTPLTLTCTVSGFTISDYDLSWVRQAPGEGLKYIGFIAIDGNPYYATWAKGRFTISKTSTTVDLKITAPTTEDTATYFCARGAGDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 24 - protein, 238 aa, Artificial Sequence
  Synthetic Construct
MDTREPTQLLGLLLLWLPGARCADVVMTQTPASVSAAVGGTVTINCQSSKNVYNNNWLSWFQQKPGQPPKLLIYYASTLASGVPSRFRGSGSGTQFTLTISDVQCDDAATYYCAGDYSSSSDNGFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 25 - protein, 454 aa, Artificial Sequence
  Synthetic Construct
METGLRWLLLVAVLKGVHCQSVEESGGRLVTPGTPLTLTCTASGFSRSSYDMSWVRQAPGKGLEWVGVISTAYNSHYASWAKGRFTISRTSTTVDLKMTSLTTEDTATYFCARGGSWLDLWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 26 - protein, 239 aa, Artificial Sequence
  Synthetic Construct
  misc_feature (3)..(3): Xaa can be any naturally occurring amino acid
MDXRAPTQLLGLLLLWLPGARCALVMTQTPASVSAAVGGTVTINCQSSQSVYDNDELSWYQQKPGQPPKLLIYALASKLASGVPSRFKGSGSGTQFALTISGVQCDDAATYYCQATHYSSDWYLTFGGGTEVVVKGFPVAPTVLLFPPSSDEVATGTVTIVCVANKYFPDVTVTWEVDGTTQTTGTENSKTPQNSADCTYNLSSTLTLTSTQYNSHKEYTCKVTQGTTSVVQSFSRKNC

SEQ ID NO: 27 - protein, 456 aa, Artificial Sequence
  Synthetic Construct
METGLRWLLLVAVLKGVQCQSVEESGGRLVTPGTPLTLTCTVSGFSLSSYDMTWVRQAPGKGLEWIGIIYASGTTYYANWAKGRFTISKTSTTVDLKVTSPTIGDTATYFCAREGAGVSMTLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 28 - protein, 236 aa, Artificial Sequence
  Synthetic Construct
MDMRAPTQLLGLLLLWLPGARCAYDMTQTPASVEVAVGGTVTIKCQASQSISTYLDWYQQKPGQPPKLLIYDASDLASGVPSRFKGSGSGTQFTLTISDLECADAATYYCQQGYTHSNVDNVFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 29 - protein, 459 aa, Artificial Sequence
  Synthetic Construct
METGLRWLLLVAVLKGVQCQSVEESGGRLVSPGTPLTLTCTASGFSLSSYDMSWVRQAPGKGLEYIGIISSSGSTYYASWAKGRFTISKTSTTVDLEVTSLTTEDTATYFCSREHAGYSGDTGHLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVGIGPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 30 - protein, 236 aa, Artificial Sequence
  Synthetic Construct
MDMRAPTQLLGLLLLWLPGARCAYDMTQTPASVEVAVGGTVAIKCQASQSVSSYLAWYQQKPGQPPKPLIYEASMLAAGVSSRFKGSGSGTDFTLTISDLECDDAATYYCQQGYSISDIDNAFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 31 - protein, 455 aa, Artificial Sequence
  Synthetic Construct
METGLRWLLLVAVLKGVQCQSVEESGGRLVTPGTPLTLTCTVSGIDLSSDGISWVRQAPGKGLEWIGIISSGGNTYYASWAKGRFTISRTSTTVDLKMTSLTTEDTATYFCARVVGGTYSIWGQGTLVTVSSASTKGPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 32 - protein, 240 aa, Artificial Sequence
  Synthetic Construct
MDTRAPTQLLGLLLLWLPGATFAQVLTQTASPVSAPVGGTVTINCQSSQSVYNNDFLSWYQQKPGQPPKLLIYYASTLASGVPSRFKGSGSGTQFTLTISDLECDDAATYYCTGTYGNSAWYEDAFGGGTEVVVKRTPVAPTVLLFPPSSAELATGTATIVCVANKYFPDGTVTWKVDGITQSSGINNSRTPQNSADCTYNLSSTLTLSSDEYNSHDEYTCQVAQDSGSPVVQSFSRKSC

SEQ ID NO: 33 - protein, 460 aa, Artificial Sequence
  Synthetic Construct
METGLRWLLLVAVLKGVQCQSVEESGGRLVTPGTPLTLTCTVSGIDLSSNAMIWVRQAPREGLEWIGAMDSNSRTYYATWAKGRFTISRTSSITVDLKITSPTTEDTATYFCARGDGGSSDYTEMWGPGTLVTVSSASTKGPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 34 - protein, 239 aa, Artificial Sequence
  Synthetic Construct
MDTRAPTQLLGLLLLWLPGATFAQAVVTQTTSPVSAPVGGTVTINCQSSQSVYGNNELSWYQQKPGQPPKLLIYQASSLASGVPSRFKGSGSGTQFTLTISDLECDDAATYYCLGEYSISADNHFGGGTEVVVKRTPVAPTVLLFPPSSAELATGTATIVCVANKYFPDGTVTWKVDGITQSSGINNSRTPQNSADCTYNLSSTLTLSSDEYNSHDEYTCQVAQDSGSPVVQSFSRKSC

SEQ ID NO: 35 - protein, 221 aa, Artificial Sequence
  Synthetic Construct
QVQLVQSGGAVVQPGRSLRLSCAASGFTFSTYGMHWVRQAPGKGLEWVAAVSYDGSNKYYADFVKGRFTISRDNPKNTLYLQMNSLRADDTAVYYCARRGGLDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAA

SEQ ID NO: 36 - protein, 110 aa, Artificial Sequence
  Synthetic Construct
LTQPPPASGTPGQQRVTISCSGSSSNIGSNTVNWYQQLPGTAPKLLIYYGNDQRPSGVPDRFSASKSGTSASLAISGLQSEDEAHYYCAAWDGSLNGGVIFGGGTKVTLG

SEQ ID NO: 37 - protein, 109 aa, Artificial Sequence
  Synthetic Construct
VTQPPSASGTPGQRVTISCSGSSSNIGTNPVNWYQQLPGTAPKLLIYTTDQRPSGVPDRFSGSKSGTSASLAISGLQSEDEADYYCAAWDDSLSGLYVFGTGTKVTVLG

SEQ ID NO: 38 - protein, 109 aa, Artificial Sequence
  Synthetic Construct
MTHTPLSLSVTPGQPASISCKSSQSLLHSDGKTYLYWYLQRPGQSPQPLIYEVSNRFSGVPDRFSGSGSGTDFTLKISRVQAEDVGVYYCMQSLQLWTFGQGTKVEIKR

SEQ ID NO: 39 - protein, 110 aa, Artificial Sequence
  Synthetic Construct
MTQSPLSLPVTLGQPASISCRSSQSLIHSDGNTYLDWFQQRPGQSPRRLIYKVSNRDSGVPDRFSGSGSGTDFTLRISRVEAEDIGVYYCMQATHWPRTFGQGTKVEIKR

SEQ ID NO: 40 - protein, 110 aa, Artificial Sequence
  Synthetic Construct
MTQSPLSLPVTLGQPASISCRSSQSLVDSAGNTFLHWFHQRPGQSPRRLIYKVSNRDSGVPDRFSGSGSGTDFTLKISRVEAEDVGVYYCMQGTHWPRTFGQGTKVEIKR

SEQ ID NO: 41 - protein, 110 aa, Artificial Sequence
  Synthetic Construct
LTQSPLSLPVTLGQPASISCKSSQSLVDSDGNTYLNWFQQRPGQSPRRLIYKVSNRDSGVPDRFSGSGSGTDFTLKISRVEAEDVGVYYCMQGTHWPRTFGQGTKVEIKR

SEQ ID NO: 42 - protein, 110 aa, Artificial Sequence
  Synthetic Construct
MTQSPLSLPVTLGQPASISCRSSQSLVHSDGNMYLNWFQQRPGQSPRRLIYKVSNRDSGVPDRFSGSGSGTDFTLKISRVEAEDVGVYYCMQATQPTWTFGQGTKLEIKR

SEQ ID NO: 43 - protein, 92 aa, Artificial Sequence
  Synthetic Construct
MTQSPSSLSASVGDRVTITCQASQDISNYLNWYQQKPGKAPKLLIYDASNLETGVPSRFSGSGSGTDFTFTISSATYYCQQTYQGTKLEIKR

SEQ ID NO: 44 - protein, 105 aa, Artificial Sequence
  Synthetic Construct
MTQSPSSLSASVGHPVTITCRASQSLISYLNWYHQKPGKAPKLLIYAASILQSGVPSRFSGSGSGTDFTLTISSLQPENFASYYCQHTDSFPRTFGHGTKVEIKR

SEQ ID NO: 45 - protein, 108 aa, Artificial Sequence
  Synthetic Construct
LTQPPSASGTPGQGVTISCRGSTSNIGNNVVNWYQHVPGSAPKLLIWSNIQRPSGIPDRFSGSKSGTSASLAISGLQSEDQAVYYCAVWDDGLAGWVFGGGTTVTVLS

SEQ ID NO: 46 - protein, 105 aa, Artificial Sequence
  Synthetic Construct
MTQAPVVSVALEQTVRITCQGDSLAIYYDFWYQHKPGQAPVLVIYGKNNRPSGIPHRFSGSSSNTDSLTITGAQAEDEADYYCNSRDSSGNHWVFGGGTNLTVLG

SEQ ID NO: 47 - protein, 111 aa, Artificial Sequence
  Synthetic Construct
LTQSPLSLPVTLGQPASISCKSNQSLVHSDGNTYLNWFQQRPGQSPRRLIYKVSNRDSGVPDRFSGSGSGTDFTLKINRVEAEDVGVYYCMQGTQWPRTFGGQGTKLDIKR

SEQ ID NO: 48 - protein, 119 aa, Artificial Sequence
  Synthetic Construct
QVQLQESGPGLVKPSETLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSS

SEQ ID NO: 49 - protein, 119 aa, Artificial Sequence
  Synthetic Construct
QVQLQESGPGLVKPSQTLSLTCTVSGFSITTGGYWWTWIRQHPGKGLEWIGYIFSSGNTNYNPSIKSLVTISVDTSKNQFSLKLSSVTAADTAVYYCARAYGKLGFDYWGQGTLVTVSS

SEQ ID NO: 50 - protein, 119 aa, Artificial Sequence
  Synthetic Construct
QVQLQESGPGLVKPSQTLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSS

SEQ ID NO: 51 - protein, 119 aa, Artificial Sequence
  Synthetic Construct
QVQLQESGPGLVKPSETLSLTCTVSGFSITTGGYWWTWIRQPPGKGLEWIGYIFSSGNTNYNPSIKSRVTISRDTSKNQFSLKLSSVTAADTAVYYCARAYGKLGFDYWGQGTLVTVSS

SEQ ID NO: 52 - protein, 119 aa, Artificial Sequence
  Synthetic Construct
QVQLQESGPGLVKPSETLSLTCTVSGFSITTGGYWWTWIRQPPGKGLEWIGYIFSSGNTNYNPSIKSRVTISVDTSKNQFSLKLSSVTAADTARYYCARAYGKLGFDYWGQGTLVTVSS

SEQ ID NO: 53 - protein, 120 aa, Artificial Sequence
  Synthetic Construct
QVQLQESGPGLVKPSETLSLTCTVSGFSITTGGYWWTWIRQPPGKGLEWIGYIFSSGNTNYNPSIKSRISITRDTSKNQFFLQLNSVTTEGDTARYYCARAYGKLGFDYWGQGTLVTVSS

SEQ ID NO: 54 - protein, 111 aa, Artificial Sequence
  Synthetic Construct
QLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 55 - protein, 111 aa, Artificial Sequence
  Synthetic Construct
LPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 56 - protein, 15 aa, Artificial Sequence
  Synthetic Construct
GGGGSGGGGSGGGGS

SEQ ID NO: 57 - protein, 247 aa, Artificial Sequence
  Synthetic Construct
QPVLTQSPSASASLGNSVKITCTLSSQHSTYTIGWYQQHPDKAPKYVMYVNSDGSHSKGDGIPDRFSGSSSGAHRYLSISNIQPEDEADYFCGSSDSSGYVFGSGTQLTVLRAAASSGGGGSGGGGSGGGGSQPVLTQSPSASASLGNSVKITCTLSSQHSTYTIGWYQQHPDKAPKYVMYVNSDGSHSKGDGIPDRFSGSSSGAHRYLSISNIQPEDEADYFCGSSDSSGYVFGSGTQLTVLRAAA

SEQ ID NO: 58 - protein, 253 aa, Artificial Sequence
  Synthetic Construct
QVQLKESGPGLVKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTLVTVSSGGGGSGGGGSGGGGSQVQLKESGPGLVKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTLVTV

SEQ ID NO: 59 - protein, 245 aa, Artificial Sequence
  Synthetic Construct
QVQLQESGPGLVKPSETLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 60 - protein, 245 aa, Artificial Sequence
  Synthetic Construct
QVQLQESGPGLVKPSQTLSLTCTVSGFSITTGGYWWTWIRQHPGKGLEWIGYIFSSGNTNYNPSIKSLVTISVDTSKNQFSLKLSSVTAADTAVYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 61 - protein, 245 aa, Artificial Sequence
  Synthetic Construct
QVQLQESGPGLVKPSQTLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 62 - protein, 245 aa, Artificial Sequence
  Synthetic Construct
QVQLQESGPGLVKPSETLSLTCTVSGFSITTGGYWWTWIRQPPGKGLEWIGYIFSSGNTNYNPSIKSRVTISRDTSKNQFSLKLSSVTAADTAVYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 63 - protein, 245 aa, Artificial Sequence
  Synthetic Construct
QVQLQESGPGLVKPSETLSLTCTVSGFSITTGGYWWTWIRQPPGKGLEWIGYIFSSGNTNYNPSIKSRVTISVDTSKNQFSLKLSSVTAADTARYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 64 - protein, 246 aa, Artificial Sequence
  Synthetic Construct
QVQLQESGPGLVKPSETLSLTCTVSGFSITTGGYWWTWIRQPPGKGLEWIGYIFSSGNTNYNPSIKSRISITRDTSKNQFFLQLNSVTTEGDTARYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 65 - protein, 245 aa, Artificial Sequence
  Synthetic Construct
QVQLQESGPGLVKPSETLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 66 - protein, 245 aa, Artificial Sequence
  Synthetic Construct
QVQLQESGPGLVKPSQTLSLTCTVSGFSITTGGYWWTWIRQHPGKGLEWIGYIFSSGNTNYNPSIKSLVTISVDTSKNQFSLKLSSVTAADTAVYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 67 - protein, 245 aa, Artificial Sequence
  Synthetic Construct
QVQLQESGPGLVKPSQTLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 68 - protein, 245 aa, Artificial Sequence
  Synthetic Construct
QVQLQESGPGLVKPSETLSLTCTVSGFSITTGGYWWTWIRQPPGKGLEWIGYIFSSGNTNYNPSIKSRVTISRDTSKNQFSLKLSSVTAADTAVYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 69 - protein, 245 aa, Artificial Sequence
  Synthetic Construct
QVQLQESGPGLVKPSETLSLTCTVSGFSITTGGYWWTWIRQPPGKGLEWIGYIFSSGNTNYNPSIKSRVTISVDTSKNQFSLKLSSVTAADTARYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 70 - protein, 246 aa, Artificial Sequence
  Synthetic Construct
QVQLQESGPGLVKPSETLSLTCTVSGFSITTGGYWWTWIRQPPGKGLEWIGYIFSSGNTNYNPSIKSRISITRDTSKNQFFLQLNSVTTEGDTARYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 71 - protein, 246 aa, Artificial Sequence
  Synthetic Construct
QVQLKESGPGLVKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTLVTVSSGGGGSGGGGSGGGGSQPVLTQSPSASASLGNSVKITCTLSSQHSTYTIGWYQQHPDKAPKYVMYVNSDGSHSKGDGIPDRFSGSSSGAHRYLSISNIQPEDEADYFCGSSDSSGYVFGSGTQLTVL

SEQ ID NO: 72 - DNA, 804 bp, Artificial Sequence
  Synthetic Construct
atggccagccccctgacaagattcctgagcctgaacctgctgctgctgggcgagtccatcatcctcggcagcggcgaggcccaagtgcaactgcaggaatccggagctgagctggccagacccggagccagcgtgaagatgtcctgtaaggcctccggctacaccttcaccaggtacaccatgcactgggtgaagcagaggcctggccagggcctggagtggatcggctacatcaatcccagcaggggctacaccaattacaatcagaagttcaaggacaaggccaccctcacaaccgataagagcagctccaccgcctacatgcaactgagcagcctgacctccgaggactccgccgtgtactactgtgccagatactacgacgaccactactgcctggactactggggccagggcaccacagtgacagtgagctccggcggcggcggctccggcggcggcggcagcggcggcggcggaagcgacattcagctgacccagagccccgctatcatgtccgccagccctggagagaaggtgaccatgacctgcagcgccagcagcagcgtgagctacatgaactggtaccagcagaagtccggcaccagccccaagaggtggatctacgacaccagcaagctggcttccggcgtgcctgcccactttagaggcagcggctccggcaccagctacagcctgaccatcagcggcatggaggctgaagacgccgccacctactactgtcagcagtggtcctccaaccccttcaccttcggcagcggcacaaagctggagatcaagaga

SEQ ID NO: 73 - protein, 268 aa, Artificial Sequence
  Synthetic Construct
MASPLTRFLSLNLLLLGESIILGSGEAQVQLQESGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTVTVSSGGGGSGGGGSGGGGSDIQLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIKR

SEQ ID NO: 74 - DNA, 810 bp, Artificial Sequence
  Synthetic Construct
atggcctcccccctcaccaggttcctgagcctgaacctgctgctgctgggcgagagcatcatcctgggaagcggcgaggctcaggtgcagctgcagcagagcggcaccgaactggtgaagcctgcctccagcgtgaagatcagctgcaaggccagcggctacacctccaccagcaactacatgcactggatcaggcagcagcccggcaatggcctggagtggatcggcaggatttaccccggcaacggcaacaccaaatacaatcagaagttcgatggcaaggccacccccaccgctgacaagtcctcctccaccgcctacatgcagctgagcagactgaccttcgaggatagcgccgtctacttctgcgctagcgcccctctggactacggaggccacatcatggacgcctggggccagggcaccacagtgacagtgagctccggaggcggcggcagcggcggcggcggcagcggcggaggcggcagcgatatccagctcacccagagccctgcctttctgagcgccagcctgggcgagaccgtgagcattgagtgcctcggctccgaggacatctacggctacctggcctggtatcagcagaagcccggcaagagcccccagctgctgatctacgtggccaacaggctgcaggatggcgtgcctagcagatttagcggcagcggcagcggaacccagtattccctcaagatcagcggcatgcagcctgaggacgagggcgactactactgcctccagggcagcaagtttcccctcaccttcggcagcggcaccaagctggagatcaaa

SEQ ID NO: 75 - protein, 270 aa, Artificial Sequence
  Synthetic Construct
MASPLTRFLSLNLLLLGESIILGSGEAQVQLQQSGTELVKPASSVKISCKASGYTSTSNYMHWIRQQPGNGLEWIGRIYPGNGNTKYNQKFDGKATPTADKSSSTAYMQLSRLTFEDSAVYFCASAPLDYGGHIMDAWGQGTTVTVSSGGGGSGGGGSGGGGSDIQLTQSPAFLSASLGETVSIECLGSEDIYGYLAWYQQKPGKSPQLLIYVANRLQDGVPSRFSGSGSGTQYSLKISGMQPEDEGDYYCLQGSKFPLTFGSGTKLEIK

SEQ ID NO: 76 - DNA, 762 bp, Artificial Sequence
  Synthetic Construct
atggagtacgccagcgacgccagcctggaccccgaggccccctggccccccgccccccgggcccgggcctgccgggtgctgccctgggccctggtggccggcctgctgctgctgctgctgctggccgccgcctgcgccgtgttcctggcctgcccctgggccgtgagcggcgcccgggccagccccggcagcgccgccagcccccggctgcgggagggccccgagctgagccccgacgaccccgccggcctgctggacctgcggcagggcatgttcgcccagctggtggcccagaacgtgctgctgatcgacggccccctgagctggtacagcgaccccggcctggccggcgtgagcctgaccggcggcctgagctacaaggaggacaccaaggagctggtggtggccaaggccggcgtgtactacgtgttcttccagctggagctgcggcgggtggtggccggcgagggcagcggcagcgtgagcctggccctgcacctgcagcccctgcggagcgccgccggcgccgccgccctggccctgaccgtggacctgccccccgccagcagcgaggcccggaacagcgccttcggcttccagggccggctgctgcacctgagcgccggccagcggctgggcgtgcacctgcacaccgaggcccgggcccggcacgcctggcagctgacccagggcgccaccgtgctgggcctgttccgggtgacccccgagatccccgccggcctgcccagcccccggagcgag

SEQ ID NO: 77 - protein, 254 aa, Artificial Sequence
  Synthetic Construct
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 78 - DNA, 804 bp, Artificial Sequence
  Synthetic Construct
atggcccccagaagagccagaggctgcagaaccctcggactgcccgctttactgctgctgttactgctgagaccccccgctaccagaggcattacttgtccccctcccatgagcgtcgagcatgccgacatctgggtgaagagctactctttatattctcgtgagaggtacatctgcaactccggcttcaagagaaaggccggcacaagctctttaaccgagtgcgtgctgaacaaggctacaaacgtggcccactggaccaccccctctttaaaatgcattcgtgatcccgctctggtgcaccagagacccgctcctccctccaccgtgaccacagccggagtgacccctcagcccgaatccctctccccctccggaaaggagcccgctgcctccagcccctccagcaacaacaccgctgccacaaccgccgctatcgtgcccggttcccagctgatgccctccaagagccccagcaccggaaccaccgagatcagcagccacgagagctcccacggaacacccagccagaccaccgccaagaattgggaactgaccgcctccgccagccatcagcctcccggcgtgtatcctcaaggtcacagcgacaccacagtggccatttccaccagcaccgtgttattatgcggactgtccgctgtgtctttactggcttgttatttaaagtctcgtcagacccctcctctggcttccgtggagatggaggccatggaggctttacccgtgacttggggaacctcctctcgtgacgaggatttagagaactgcagccatcatttatga

SEQ ID NO: 79 - protein, 267 aa, Artificial Sequence
  Synthetic Construct
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 80 - DNA, 1671 bp, Artificial Sequence
  Synthetic Construct
gctatccccgctcccaccgacctgaaattcacccaggtgacccccaccagcctgagcgctcaatggaccccccccaatgtgcagctgacaggctacagggtgagagtgacccccaaggaaaaaaccggacccatgaaggagatcaatctggcccctgacagctccagcgtcgtggtctccggcctgatggtggctacaaaatacgaggtgtccgtctacgccctcaaagataccctgaccagcaggcctgcccaaggagtggtgaccaccctggaaaatgtctccccccctaggagagccagggtcaccgatgccacagaaaccaccattaccatctcctggaggaccaagaccgaaaccattaccggattccaggtggatgctgtccccgccaacggacaaacacctattcaaaggacaatcaaacccgacgtgagatcctacaccatcaccggcctccagcccggcaccgactacaaaatctatctgtataccctgaacgacaacgctagaagctcccctgtggtgatcgacgctagcaccgccatcgatgcccctagcaatctgaggtttctggccaccacacctaattccctgctcgtgtcctggcagcctcccagggctaggatcaccggctacattatcaagtacgagaaacccggctccccccctagagaagtggtgcctaggcccaggcctggagtcaccgaggctaccattaccggcctggagcctggaaccgagtacaccatctatgtcatcgctctcaagaacaaccagaagtccgaacccctcattggcaggaagaagaccggcggcggcggctccggaggaggcggctccggcggcggcggaagcgctattcctgcccctaccgatctgaagttcacccaagtgacacctacctccctgtccgcccagtggaccccccccaacgtgcagctcaccggctatagggtcagggtgacccccaaagagaagaccggacccatgaaagagatcaacctcgctcctgactccagcagcgtcgtggtgagcggcctgatggtggccaccaagtacgaagtgagcgtctacgctctgaaggacaccctgaccagcagacccgcccagggagtggtgaccacactggagaacgtcagccctcccagaagggctagagtgacagacgccacagagaccaccattaccattagctggaggaccaaaaccgagaccattacaggattccaggtcgacgctgtccccgctaacggacaaacccccatccagaggaccatcaagcccgatgtgaggagctataccatcaccggactccaacccggcacagactacaagatctacctgtacaccctgaatgacaatgccaggagctcccccgtggtcattgacgcctccaccgccattgacgctcctagcaacctgaggttcctggctaccacccccaacagcctcctggtctcctggcagccccccagagctagaattaccggatacatcatcaagtatgaaaaacctggcagcccccctagggaggtcgtgcctaggcctagacctggcgtgacagaggctacaattaccggcctggagcccggcacagaatacaccatttacgtcatcgctctcaaaaacaaccagaaatccgagcccctcatcggcaggaaaaagacc

SEQ ID NO: 81 - protein, 513 aa, Artificial Sequence
  Synthetic Construct
MKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTGGGGSGGGGSGGGGSAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKT

SEQ ID NO: 82 - DNA, 1293 bp, Artificial Sequence
  Synthetic Construct
atgcccaacccccggcccggcaagcccagcgcccccagcctggccctgggccccagccccggcgccagccccagctggcgggccgcccccaaggccagcgacctgctgggcgcccggggccccggcggcaccttccagggccgggacctgcggggcggcgcccacgccagcagcagcagcctgaaccccatgccccccagccagctgcagctgcccaccctgcccctggtgatggtggcccccagcggcgcccggctgggccccctgccccacctgcaggccctgctgcaggaccggccccacttcatgcaccagctgagcaccgtggacgcccacgcccggacccccgtgctgcaggtgcaccccctggagagccccgccatgatcagcctgaccccccccaccaccgccaccggcgtgttcagcctgaaggcccggcccggcctgccccccggcatcaacgtggccagcctggagtgggtgagccgggagcccgccctgctgtgcaccttccccaaccccagcgccccccggaaggacagcaccctgagcgccgtgccccagagcagctaccccctgctggccaacggcgtgtgcaagtggcccggctgcgagaaggtgttcgaggagcccgaggacttcctgaagcactgccaggccgaccacctgctggacgagaagggccgggcccagtgcctgctgcagcgggagatggtgcagagcctggagcagcagctggtgctggagaaggagaagctgagcgccatgcaggcccacctggccggcaagatggccctgaccaaggccagcagcgtggccagcagcgacaagggcagctgctgcatcgtggccgccggcagccagggccccgtggtgcccgcctggagcggcccccgggaggcccccgacagcctgttcgccgtgcggcggcacctgtggggcagccacggcaacagcaccttccccgagttcctgcacaacatggactacttcaagttccacaacatgcggccccccttcacctacgccaccctgatccggtgggccatcctggaggcccccgagaagcagcggaccctgaacgaaatctaccactggttcacccggatgttcgccttcttccggaaccaccccgccacctggaagaacgccatccggcacaacctgagcctgcacaagtgcttcgtgcgggtggagagcgagaagggcgccgtgtggaccgtggacgagctggagttccggaagaagcggagccagcggcccagccggtgcagcaaccccacccccggcccc

SEQ ID NO: 83 - protein, 431 aa, Artificial Sequence
  Synthetic Construct
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 84 - DNA, 1653 bp, Artificial Sequence
  Synthetic Construct
atggccgcccccgccctgagctggcggctgcccctgctgatcctgctgctgcccctggccaccagctgggccagcgccgccgtgaacggcaccagccagttcacctgcttctacaacagccgggccaacatcagctgcgtgtggagccaggacggcgccctgcaggacaccagctgccaggtgcacgcctggcccgaccggcggcggtggaaccagacctgcgagctgctgcccgtgagccaggccagctgggcctgcaacctgatcctgggcgcccccgacagccagaagctgaccaccgtggacatcgtgaccctgcgggtgctgtgccgggagggcgtgcggtggcgggtgatggccatccaggacttcaagcccttcgagaacctgcggctgatggcccccatcagcctgcaggtggtgcacgtggagacccaccggtgcaacatcagctgggagatcagccaggccagccactacttcgagcggcacctggagttcgaggcccggaccctgagccccggccacacctgggaggaggcccccctgctgaccctgaagcagaagcaggagtggatctgcctggagaccctgacccccgacacccagtacgagttccaggtgcgggtgaagcccctgcagggcgagttcaccacctggagcccctggagccagcccctggccttccggaccaagcccgccgccctgggcaaggacaccatcccctggctgggccacctgctggtgggcctgagcggcgccttcggcttcatcatcctggtgtacctgctgatcaactgccggaacaccggcccctggctgaagaaggtgctgaagtgcaacacccccgaccccagcaagttcttcagccagctgagcagcgagcacggcggcgacgtgcagaagtggctgagcagccccttccccagcagcagcttcagccccggcggcctggcccccgagatcagccccctggaggtgctggagcgggacaaggtgacccagctgctgctgcagcaggacaaggtgcccgagcccgccagcctgagcagcaaccacagcctgaccagctgcttcaccaaccagggctacttcttcttccacctgcccgacgccctggagatcgaggcctgccaggtgtacttcacctacgacccctacagcgaggaggaccccgacgagggcgtggccggcgcccccaccggcagcagcccccagcccctgcagcccctgagcggcgaggacgacgcctactgcaccttccccagccgggacgacctgctgctgttcagccccagcctgctgggcggccccagcccccccagcaccgcccccggcggcagcggcgccggcgaggagcggatgccccccagcctgcaggagcgggtgccccgggactgggacccccagcccctgggcccccccacccccggcgtgcccgacctggtggacttccagcccccccccgagctggtgctgcgggaggccggcgaggaggtgcccgacgccggcccccgggagggcgtgagcttcccctggagccggccccccggccagggcgagttccgggccctgaacgcccggctgcccctgaacaccgacgcctacctgagcctgcaggagctgcagggccaggaccccacccacctggtg

SEQ ID NO: 85 - protein, 551 aa, Artificial Sequence
  Synthetic Construct
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 86 - DNA, 675 bp, Artificial Sequence
  Synthetic Construct
atggtgacccacagcaagttccccgccgccggcatgagccggcccctggacaccagcctgcggctgaagaccttcagcagcaagagcgagtaccagctggtggtgaacgccgtgcggaagctgcaggagagcggcttctactggagcgccgtgaccggcggcgaggccaacctgctgctgagcgccgagcccgccggcaccttcctgatccgggacagcagcgaccagcggcacttcttcaccctgagcgtgaagacccagagcggcaccaagaacctgcggatccagtgcgagggcggcagcttcagcctgcagagcgacccccggagcacccagcccgtgccccggttcgactgcgtgctgaagctggtgcaccactacatgcccccccccggcgcccccagcttccccagcccccccaccgagcccagcagcgaggtgcccgagcagcccagcgcccagcccctgcccggcagccccccccggcgggcctactacatctacagcggcggcgagaagatccccctggtgctgagccggcccctgagcagcaacgtggccaccctgcagcacctgtgccggaagaccgtgaacggccacctggacagctacgagaaggtgacccagctgcccggccccatccgggagttcctggaccagtacgacgcccccctg

SEQ ID NO: 87 - protein, 225 aa, Artificial Sequence
  Synthetic Construct
MVTHSKFPAAGMSRPLDTSLRLKTFSSKSEYQLVVNAVRKLQESGFYWSAVTGGEANLLLSAEPAGTFLIRDSSDQRHFFTLSVKTQSGTKNLRIQCEGGSFSLQSDPRSTQPVPRFDCVLKLVHHYMPPPGAPSFPSPPTEPSSEVPEQPSAQPLPGSPPRRAYYIYSGGEKIPLVLSRPLSSNVATLQHLCRKTVNGHLDSYEKVTQLPGPIREFLDQYDAPL